(19) United States
(12) Patent Application Publication
(10) Pub. No.: US 2015/0067920 A1
(43) Pub. Date: Mar. 5, 2015

(54) PLANTS WITH INCREASED STRESS TOLERANCE

(71) Applicants: University of Georgia Research Foundation, Inc., Athens, GA (US); Michigan Technological University, Houghton, MI (US)

(72) Inventors: Chung-Jui Tsai, Athens, GA (US); Yinan Yuan, Houghton, MI (US)

(21) Appl. No.: 14/335,434

(22) Filed: Jul. 18, 2014

Related U.S. Application Data
(60) Provisional application No. 61/856,464, filed on Jul. 19, 2013.

(51) Int. Cl. C12N 15/82; C12N 9/88

(52) U.S. Cl. CPC C12N 15/8271; C12N 9/88; C12Y 401/03027
USPC 800/288; 800/298; 435/419; 435/320.1

(57) ABSTRACT

Provided herein are plants with increase stress tolerance and methods of making same.

This application claims the benefit of U.S. Provisional Application No. 61/856,464, filed Jul. 19, 2013, which is hereby incorporated by reference herein in its entirety.

### STATEMENT REGARDING FEDERALLY FUNDED RESEARCH

This invention was made with government funding under National Science Foundation Grant Numbers DBI-0421756 and DBI-0836433. The government has certain rights in this invention.

### BACKGROUND

Environmental stresses cause major economic losses in agriculture and forestry by adversely affecting the yield and quality of plants. Some of the most commonly encountered environmental stresses are disease, heat stress, freeze stress, drought and heavy metal stress, to name a few. Therefore, there is a need for increasing stress tolerance in plants.

### SUMMARY

Provided herein is a plant, comprising a heterologous expression cassette, wherein the expression cassette comprises a promoter operably linked to a polynucleotide, wherein the polynucleotide encodes a fusion polypeptide comprising a chloroplast targeting peptide and a single enzyme that catalyzes the conversion of chorismate to salicylic acid.

In some embodiments, the single enzyme catalyzes the direct conversion of chorismate to salicylic acid.

In some embodiments, the single enzyme is a bacterial salicylic acid synthase. In some embodiments, the bacterial salicylic acid synthase is from a bacteria selected from the group consisting of a Yersinia, Mycobacterium tuberculosis, Escherichia coli, Klebsiella, Enterobacter, Citrobacter and Raoultella. In some embodiments, the enzyme is Yersinia enterocolitica Irp9.

In some embodiments, the amount of SA in the plant is increased compared to a control plant that does not comprise the expression cassette. In some embodiments, the plant has enhanced stress tolerance compared to a control plant that does not comprise the expression cassette. In some embodiments, the plant's growth is not significantly affected, as compared to a control plant that does not comprise the expression cassette. In some embodiments, the plant has increased disease resistance, drought tolerance, heat tolerance or heavy metal tolerance compared to a control plant that does not comprise the expression cassette

In some embodiments, the plant is a Populus plant.

Also provided is a plant cell or plant seed comprising a heterologous expression cassette, wherein the expression cassette comprises a promoter operably linked to a polynucleotide, wherein the polynucleotide encodes a fusion polypeptide comprising a chloroplast targeting peptide and a single enzyme that catalyzes the conversion of chorismate to salicylic acid.

In some embodiments, the single enzyme catalyzes the direct conversion of chorismate to salicylic acid.

In some embodiments, the single enzyme is a bacterial salicylic acid synthase. In some embodiments, the bacterial salicylic acid synthase is from a bacteria selected from the group consisting of a Yersinia, Mycobacterium tuberculosis, Escherichia coli, Klebsiella, Enterobacter, Citrobacter and Raoultella. In some embodiments, the enzyme is Yersinia enterocolitica Irp9.

In some embodiments, the amount of SA in the plant cell or plant seed is increased compared to a control plant cell or seed that does not comprise the expression cassette. In some embodiments, the plant cell or plant seed has enhanced stress tolerance compared to a control plant cell or plant seed that does not comprise the expression cassette.

In some embodiments, the plant cell or plant seed is from a Populus plant.

Further provided are transgenic plants comprising any of the plant cells described herein.

Further provided are methods of making a plant comprising a heterologous expression cassette, wherein the expression cassette comprises a promoter operably linked to a polynucleotide, wherein the polynucleotide encodes a fusion polypeptide comprising a chloroplast targeting peptide and a single enzyme that catalyzes the conversion of chorismate to salicylic acid. In some embodiments, the method comprises transforming a plant cell or a plant seed with a heterologous expression cassette, wherein the expression cassette comprises a promoter operably linked to a polynucleotide, wherein the polynucleotide encodes a fusion polypeptide comprising a chloroplast targeting peptide and a single enzyme that catalyzes the conversion of chorismate to salicylic acid; and regenerating a transgenic plant from said transformed plant cell or plant seed.

Also provided are plant cells or seeds from the plants produced by any of the methods described herein. Further provided are plants produced by any of the methods described herein.

Further provided is a heterologous expression cassette, wherein the expression cassette comprises a promoter operably linked to a polynucleotide, wherein the polynucleotide encodes a fusion polypeptide comprising a chloroplast targeting peptide and a single enzyme that catalyzes the conversion of chorismate to salicylic acid. In some embodiments a vector comprises any of the expression cassettes described herein. In some embodiments, a host cell comprises the vector.

### BRIEF DESCRIPTION OF THE DRAWINGS

FIG. 1 is a schematic of salicylic acid (SA) biosynthetic pathways and their downstream metabolites. Reactions catalyzed by Irp9 and NahG are shown in bold. Dashed arrows indicate multi-step pathways, and question marks denote unresolved biosynthetic steps.

FIG. 2 shows a screening analysis of independent transgenic lines. (A) to (C) qRT-PCR analysis of FD-Irp9 (A), Irp9 (B) and NahG (C) transgenic lines. Bars are means±SD of two technical replicates. Expression in WT was below detection. (D) to (E) Relative abundance of SA metabolites in representative transgenic and WT plants. Shown are extracted ion chromatograms (D) and mass spectral fragmentation patterns (E) of gentisic acid glucoside (1), SA glucoside (2), SA glucose ester (3), putative syringic acid glucoside (4), and SA (5). Traces are provided for FD-Irp9 and NahG lines, as well as for WT and Irp9.

FIG. 3 shows leaf photosynthetic characteristics under varying growth temperatures. Net photosynthesis, stomatal conductance and transpiration were measured in young (LPI-5) and mature (LPI-10) source leaves of WT and two independent lines each from the FD-Irp9 (F52 and F55) and NahG (N24 and N31) transgenics. Error bars are SD of 3-10 biological replicates. The effects of treatment or genotype were assessed by repeated measures ANOVA and indicated by p values. Significant genotypic effects were further analyzed by pairwise comparison between WT and individual transgenic lines, as denoted by asterisks (***p≦0.01; **0.01<p≦0.05; *0.05<p≦0.1).

FIG. 4 shows growth of wild-type and transgenic Populus. (A) to (B) Height growth of plants maintained under ambient temperatures (A) or ambient temperatures interrupted by a 1-week exposure to elevated temperatures (B). Data represent means±SD, n=8-9 for WT, 7-9 for Irp9 (combined from two independent lines), 2-4 for FD-Irp9 (F) lines and 3-5 for NahG (N) lines. Height increment per unit time did not differ significantly among genotypes based on repeated measures ANOVA (p=0.52 and 0.73 for A and B, respectively). (C) to (D) Height growth (C) and diameter increment (D) of plants maintained in a greenhouse over one summer month. n=6 for WT, n=3 for Irp9, n=2-5 for F lines and n=3-4 for N lines. Height increment per unit time did not differ significantly based on repeated measures ANOVA (p=0.37). No significant difference was found for diameter increment between WT and individual transgenic lines during the monitoring period based on the two-sample t-test.

FIG. 5 shows electrolyte leakage analysis. (A) to (B) Leaf discs were sampled one week after heat treatment (A) or recovery (B) from the two-chamber heat experiment. Data represent means±SE using pooled transgenic lines and/or biological replicates (n=7-12). Statistical significance between normal (NT) and high temperature (HT) conditions was evaluated by the two-sample t-test (***p≦0.001; **0.001<p≦0.01, *0.01<p≦0.05) (C) Leaf discs from greenhouse-grown WT and FD-Irp9 (F10) plants were incubated at 25° C., 37° C. or 50° C. for the indicated time prior to electroconductivity measurements (n=5). Repeated measures ANOVA showed a significant temperature effect (p≦0.001) as expected, but the differences between genotype were not significant (p=0.7).

FIG. 6 shows the relative abundance of SA and SA-related conjugates. Samples are color-coded by plant group. Lighter and darker bars denote normal and high temperatures, respectively. Values are means±SD of n=7-10 for WT and Irp9 plants, n=2-3 for FD-Irp9 (F) lines, and n=3-5 for NahG (N) lines. Statistical significance between WT and individual transgenic lines was evaluated by the two-sample t-test (***p≦0.001; **0.001<p≦0.01, *0.01<p≦0.05).

FIG. 7 is a regression analysis between total SA and total PGs, total chlorogenic acids or individual chlorogenic acid isomers. Data from all transgenic lines and/or biological replicates within each genotype were used for the analysis (n=7-9). The NahG group was excluded from the analysis, as the total SA levels were relatively invariable. The total SA levels are represented as the sum of the normalized peak area of SA, SAG, SA glucose ester and gentisic acid glucoside; total PGs as the sum of salicin, salicortin and tremulacin; and total CGAs as the sum of 3-CQA, 4-CQA and 5-CQA. Open and filled symbols were from normal and high temperature conditions, respectively. CGA, chlorogenic acid, CQA, caffeoylquinic acid.

FIG. 8 shows a metabolite correlation network in response to SA and temperature manipulation. Significant associations (absolute values of Pearson correlation coefficients≧0.5, p≦0.05) from all pairwise comparisons across all samples are visualized in Cytoscape. Compounds are numbered to facilitate cross-referencing with FIG. 9.

FIG. 9 shows a metabolite and gene network correlation. (A) Hierarchical clustering analysis of relative metabolite abundance across genotypes and treatments. Metabolite numbering and grouping as in FIG. 8. (B) Heatmap illustration of correlations between metabolites and module eigengenes obtained from gene network analysis. Modules are shown on top. Scale bars depict the correlation strength and directionality (positive or negative) for both panels. HT, high temperature, NT, normal temperature.

FIG. 10 shows gene expression changes in response to transgenic manipulation. (A) to (B) Venn diagrams of genes significantly changed in the transgenics relative to WT under normal (A) or high (B) temperatures. (C) to (D) Breakdown of up-regulated (black bars, positive numbers) and down-regulated (grey bars, negative numbers) under normal (C) or high (D) temperatures.

FIG. 11 shows gene expression responses to heat treatment in WT and transgenic lines. (A) Venn diagram of genes significantly affected by heat in the four genotypes. (B) to (G) Representative patterns of gene expression profiles based on clustering analysis of gene subsets from A. The average expression profiles of the four genotypes are shown in each panel, with gene number listed in parentheses. Genes exhibiting significant differences are shaded according to the Venn diagram in A. Open and filled circles denote normal and high temperature treatments, respectively. Y-axis in G is for panels B-G. (H) Venn diagram of genes significantly changed in F10 relative to WT (SA effect) or by heat treatment in WT (heat effect).

FIG. 12 is a GO enrichment analysis of differentially expressed genes. (A) GO enrichment patterns of genes significantly affected by transgenic manipulation under normal (NT) or high temperature (HT) conditions. (B) GO enrichment patterns of heat-responsive genes in WT and transgenic lines. Significantly enriched GO terms from up (u)- or down (d)-regulated genes of each genotype/treatment comparison were subjected to clustering analysis using the negative log 10 transformed p values and visualized in heatmaps according to the color scale. Genotype/treatment comparisons are arranged in columns, while enrichment significance of GO terms is shown in rows.

FIG. 13 shows the properties of the weighted gene correlation network. (A) Distribution of the node degree of the reconstructed network follows a power-law behavior. (B) Module assignment using the dynamic tree cut method. (C) to (J) Expression profiles of module gene members and module eigengene are shown by heatmap (top) and bar graph (bottom), respectively, in each panel for representative modules.

FIG. 14 shows the topology of the weighted gene correlation network. (A) The inferred gene network. (B) Distribution of the top 5% most highly connected hub genes from the entire network. (C) Distribution of the top 5% most connected nodes from each module. Non-hub nodes in B and C are shown in grey.

FIG. 15 shows comparisons between the WT and FD-Irp9 subnetworks. (A) to (B) Module assignment for the WT (A) and FD-Irp9 (B) subnetworks. Corresponding module assignment from the other subnetwork is also shown. (C) to (E) Module similarity analysis between the two subnetworks (C), or between the total network with the WT subnetwork (D) or the FD-Irp9 subnetwork (E). Numbers of overlapping genes for all pairwise module analysis are shown. (F) to (H) Heatmap plots of hub gene similarity among networks. The top 100 most highly connected hub genes from the total network (F) or the FD-Irp9 (G) or WT (H) subnetwork were obtained and their connectivity in the corresponding WT (upper half) or FD-Irp9 subnetwork (lower half) is shown. Arrows indicate decreasing hub gene ranking (connectivity) and color denotes the degree of connectivity (darker color=higher connectivity).

FIG. 16 is a graphic representation of potential drivers in the SA-modulated gene network. (A) Scatterplot of differential expression and differential connectivity between the WT and FD-Irp9 subnetworks. Nodes are colored by their module assignment in the total network. Dashed lines denote the arbitrary cutoffs for the two parameters. Potential drivers that exhibited increased expression and increased network connectivity in the FD-Irp9 plants relative to WT are found on the top right corner (144 genes plus the SAG node). (B) to (C) Expression responsiveness of the driver genes to oxidative stress treatments based on meta-analysis of published Populus leaf microarray data. Gene expression differences between stressed samples and their respective controls were assessed by fold-change (FC, B) or statistical significance (p-value, C). Horizontal lines depict three arbitrary thresholds in each panel. Stress experiments that triggered significant changes in driver gene expression are shaded, and include SA and/or heat (nos. 1-4, this study), wounding (no. 14), drought (no. 49), pathogen infection (no. 52) and ozone (no. 53).

FIG. 17 shows SAG and NRX1 transcript abundance. (A) NRX1 probe hybridization signals (solid lines) correlated with SAG abundance (dashed line) across genotypes and treatments. Only the best-matching NRX1 name is shown for each probe The Pearson correlation coefficient between SAG and the respective NRX1 probe is shown in parentheses. (B) Relative NRX1 transcript abundance obtained by qRT-PCR (vertical bars) superimposed over SAG levels (dashed line). The NRX1 transcript levels (left axis) are shown as means±SD of 2-3 biological replicates. The primers were designed based on consensus sequence of all Populus NRX1 members. SAG data (right axis) is from FIG. 4. NT, normal temperature; HT, high temperature.

FIG. 18 is a qRT-PCR analysis of representative WRKY and RLK genes identified by microarray analysis to exhibit SA-dependent expression. Data represent means±SD of 2-3 biological replicates. NT, normal temperature; HT, high temperature.

FIG. 19 shows the expression responsiveness of the hub genes to oxidative stress treatments based on meta-analysis of published Populus leaf microarray data. Gene expression differences between stressed samples and their respective controls were assessed by fold-change (FC, top panel) or statistical significance (p-value, bottom panel). Horizontal lines depict three arbitrary thresholds in each panel. Stress experiments are highlighted as in FIG. 17, including SA and/or heat (nos. 1-4, this study), wounding (no. 14), drought (no. 49), pathogen infection (no. 52) and ozone (no. 53). Note the overall weaker responses of the hub genes compared to the driver genes shown in FIG. 17.

FIG. 20 shows that transgenic Arabidopsis expressing the plastidic FD-Irp9 gene accumulated elevated levels of SA-glucoside (SAG) with normal growth. SAG level is typically undetectable in WT Arabidopsis, but is present at very high levels in a representative transgenic line (F-2421) (n=3 biological replicates).

FIG. 21 shows that plant growth was not affected in transgenic FD-Irp9 Arabidopsis. The top and middle panels show young WT and FD-Irp9 transgenic Arabidopsis seedlings. The bottom panel shows flowering WT, sid2-2 mutant and FD-Irp9 transgenic Arabidopsis plants. The sid2-2 mutant has a defect in SA biosynthesis.

FIG. 22 also shows that hyperaccumulation of SA can be achieved in transgenic Arabidopsis from an additional independent transformation experiment Three FD-Irp9 transgenic lines with the highest levels of SA metabolites are shown. SA=salicylic acid, SAG=SA glucoside, SGE=SA glucose ester, GAG=gentisic acid glucoside.

FIG. 23 shows that under severe salt stress (300 mM for 3 days), electrolyte leakage of WT and NahG leaves increased by more than 4-fold relative to unstressed leaves. The increase in high-SA plants was ˜2-fold, resulting in significantly lower levels of electrolyte leakage in high-SA plants than in WT and NahG plants.

FIG. 24 shows that SA-hyperaccumulating plants had fewer leaves than the WT and NahG plants (top panel), but similar number of bolts (flowering stems) (bottom panel) under normal growth conditions. Salt stress had negative effects on growth (number of leaves (top panel) and bolts (bottom panel)) in all plants, but the effects were stronger on WT and NahG plants than on high-SA lines.

FIG. 25 shows that transgenic soybean with increased SA levels were produced by expressing the FD-Irp9 construct in soybean.

### DETAILED DESCRIPTION

Salicylic acid (SA) is a phytohormone regulating many aspects of plant growth and adaptation, including photosynthesis, transpiration, thermogenesis, oxidative stress response and disease resistance. The plants and methods provided herein are based, in part, on the discovery that SA can be increased in plants in order to increase stress tolerance without compromising plant growth.

### Plants

Provided herein is a plant comprising a heterologous expression cassette, wherein the expression cassette comprises a promoter operably linked to a polynucleotide, wherein the polynucleotide encodes a fusion polypeptide comprising a chloroplast targeting peptide and a single enzyme that catalyzes the conversion of chorismate to salicylic acid.

Further provided is a plant cell or a plant seed comprising a heterologous expression cassette, wherein the expression cassette comprises a promoter operably linked to a polynucleotide, wherein the polynucleotide encodes a fusion polypeptide comprising a chloroplast targeting peptide and a single enzyme that catalyzes the conversion of chorismate to salicylic acid.

As used throughout, a plant includes whole plants, derivatives or portions thereof including, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

The plants described herein have an increased amount of salicylic acid compared to a control plant. As used herein, a control plant can be a plant that does not comprise a heterologous expression cassette comprising a chloroplast targeting peptide and a single enzyme that catalyzes the conversion of chorismate to salicylic acid or a plant transformed with an empty vector. The control plant can be a plant from the same species that has been cultivated under the same conditions as the plant comprising the heterologous expression cassette. The increase in salicylic acid and or salicylic acid conjugates can be at least about a 5-fold, 10-fold, 25-fold, 50-fold, 100-fold, 250-fold, 500-fold, 1000-fold, 2000-fold, 3000-fold, 4000-fold, 5,000 fold increase or greater when compared to a control plant. Examples of salicylic acid conjugates include, but are not limited to, SA glucosides, gentisic acid glucosides and SA glucose esters. The increase in salicylic acid and/or salicylic acid conjugates is an increase that results in an amount of salicylic acid and/or salicylic acid conjugates expressed in the plant that is sufficient to increase stress tolerance in the plant without significantly affecting plant growth.

The plants described herein also have increased or enhanced stress tolerance as compared to a control plant. Enhanced stress tolerance refers to an increase in the ability of a plant to decrease or prevent symptoms associated with one or more stresses. The stress can be a biotic stress that occurs as a result of damage done to plants by other living organisms such as a pathogen (for example, bacteria, viruses, fungi, parasites), insects, nematodes, weeds, cultivated or native plants. The stress can also be an abiotic stress such as extreme temperatures (high or low), high winds, drought, salinity, chemical toxicity, oxidative stress, flood, tornadoes, wildfires, radiation and exposure to heavy metals. Therefore, increased stress tolerance can be, but is not limited to, an increase in disease resistance, an increase in drought resistance, an increase in heat tolerance, an increase in low temperature tolerance, and/or an increase in heavy metal tolerance, to name a few.

Further, the plants described herein have increased stress tolerance and normal growth as compared to a control plant. In other words, the growth of the plant is not significantly affected by increased salicylic acid production. Further, increased salicylic acid production in the plant is not toxic to the plant. One of skill in the art would know how to measure plant growth, for example, by using the methods set forth in the Examples. Other methods include, but are not limited to measuring the rate of leaf production, stem elongation, flower production, seed production, fruit production, and/or germination, to name a few. Although some developmental stages may be delayed or slowed, as long as plants do not exhibit dwarfism or significant growth retardation, and are able to reproduce, they are considered as not having negative growth consequences.

Plants with increased or enhanced stress tolerance can be selected or identified in several ways. One of ordinary skill in the art will recognize that the following methods are but a few of the possibilities. One of skill in the art will also recognize that stress responses of plants vary depending on many factors, including the type of stress and plant used. Generally, enhanced stress tolerance is measured by the reduction or elimination of symptoms associated with a particular stress when compared to a control plant. This reduction does not have to be complete, as this reduction can be about a 10, 20, 30, 40, 50, 60, 70, 80, 90 or 100% reduction when compared to a control plant.

For example, one method of selecting plants with increased disease resistance is to determine resistance of a plant to a specific plant pathogen (see, e.g., Agrios, Plant Pathology (Academic Press, San Diego, Calif.) (1988)). Enhanced resistance is measured by the reduction or elimination of disease symptoms when compared to a control plant. In some cases, however, enhanced resistance can also be measured by the production of the hypersensitive response (HR) of the plant (see, e.g., Staskawicz et al. Science 268(5211): 661-7 (1995)). Plants with enhanced disease resistance can produce an enhanced hypersensitive response relative to control plants.

In another example, one of skill in the art can select plants with increased abiotic (e.g., drought, high or low temperatures, heavy metal, UV, salt) resistance by determining the rates of photosynthesis and stomatal conductance of a plant under stress conditions (See, for example, Hozain et al. Tree Physiology 30: 32-44 (2010); Frost et al. PLoS One 7(8):e44467 (2012)). Tolerance to stresses can also be gauged by production of reactive oxygen species (ROS), by increased expression of marker genes (such as genes encoding heat-shock protein in the case of heat tolerance), or by electrolyte leakage assays of the membrane (Wahid et al. Environmental and Experimental Botany 61(3):199-223(2007); Bajji et al. Plant Growth Regulation 36:61-70 (2002)).

### Enzymes

In the plants and methods disclosed herein, any single enzyme that catalyzes the conversion of chorismate to salicylic acid can be used as long as the single enzyme provides all of the enzymatic activity necessary to convert chorismate to salicylic acid. For example, the enzyme can be a salicylic acid synthase. The enzyme can catalyze the direct conversion of chorismate to salicylic acid, i.e., in one step, or the enzyme can catalyze the indirect conversion of chorismate to salicylic acid by converting chorismate to one or more intermediates, for example, isochorismate, and then converting the intermediate to salicylic acid. If the enzyme converts chorismate to salicylic acid via an intermediate, the enzyme can be a bifunctional synthase that possesses more than one type of enzymatic activity, for example, a first enzymatic activity to convert chorismate to an intermediate and a second enzymatic activity to convert the intermediate to salicylic acid. For example, the single enzyme can have isochorismate synthase activity to convert chorismate to isochorismate and isochorismate pyruvate lyase activity to convert isochorismate to salicylic acid. Whether the single enzyme directly or indirectly converts chorismate to salicylic acid, all of the activity necessary for producing salicylic acid in the plant resides in one enzyme, without the need for additional enzymes.

Therefore, although the expression cassettes disclosed herein can comprise one or more copies of a first polynucleotide encoding a single enzyme that catalyzes the conversion of chorismate to salicylic acid, the expression cassette does not comprise a second polynucleotide sequence encoding a second enzyme involved in the production of salicylic acid, wherein the second enzyme does not possess all of the activity necessary to convert chorismate to salicylic acid. In particular, the expression cassettes provided herein do not comprise polynucleotide sequences encoding two or more enyzmes that, in combination, convert chorismate to salicylic acid, unless each of the two or more enzymes individually can convert chorismate to salicylic acid. Therefore, expression cassettes comprising a polynucleotide sequence that encodes a fusion polypeptide, wherein the fusion polypeptide comprises an isochorismate synthase and an isochorismate lyase, are specifically excluded. In other examples, the expression cassettes disclosed herein do not comprise a polynucleotide encoding entC (isochorismate synthase from E. coli), a polynucleotide encoding pmsB (an isochorismate pyruvate lyase from Psuedomonas fluorescens), a polynucleotide encoding pchA (an isochorismate synthase from Pseudomonas aeruginosa) or a polynucleotide encoding pchB (an isochorismate pyruvate lyase from Pseudomonas aeruginosa). It is understood that, one or more expression cassettes can comprise a polynucleotide encoding one or more single enzymes that convert chorismate to salicylic acid. The enzymes can be from the same species or from a different species, as long as each of the one or more single enzymes individually has all of the enzymatic activity necessary to convert chorismate to salicylic acid.

The enzyme can be a salicylic acid synthase from any species, for example, a bacterial salicylic acid synthase from Yersinia, Mycobacterium tuberculosis, Escherichia coli, Klebsiella, Enterobacter, Citrobacter and Raoultella. Examples of polypeptide sequences for salicylic acid synthases from Klebsiella oxytoca E718 (SEQ ID NO: 1), Raoultella ornithinolytica B6 (SEQ ID NO: 2), Enterobacter hormaechei (SEQ ID NO: 3), Citrobacter koseri ATCC BAA-895 (SEQ ID NO: 4), Klebsiella pneumoniae subsp. pneumoniae 1084 (SEQ ID NO: 5), Yersinia enterocolitica (Irp9) (SEQ ID NO: 6), Yersinia pestis D182038 (SEQ ID NO: 7), Yersinia pseudotuberculosis PB1/+ (SEQ ID NO: 8) and Escherichia coli ABU 83972 (SEQ ID NO: 9) are provided and aligned below. As shown below, these sequences are highly conserved and comprise three key residues (E240, H321 and Y372, highlighted below) within the inferred Yersinia enterolitica Irp9 active site (Kerbarh et al. J. Mol. Biol. 187: 5061-5066 (2006)) that are conserved among the sequences. The GenBank Accession No. for each of the protein sequences is also provided.

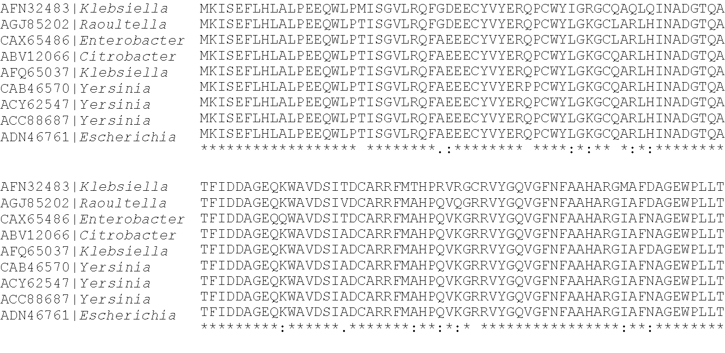

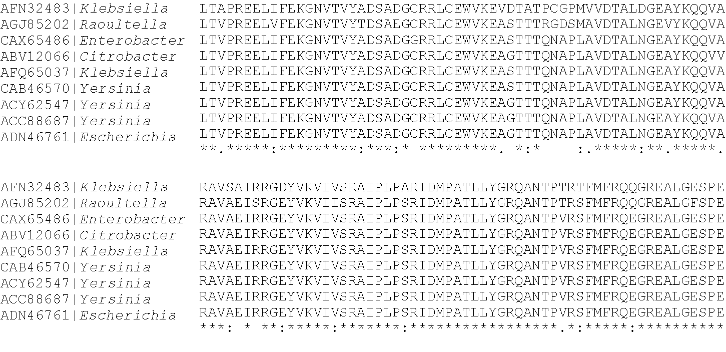

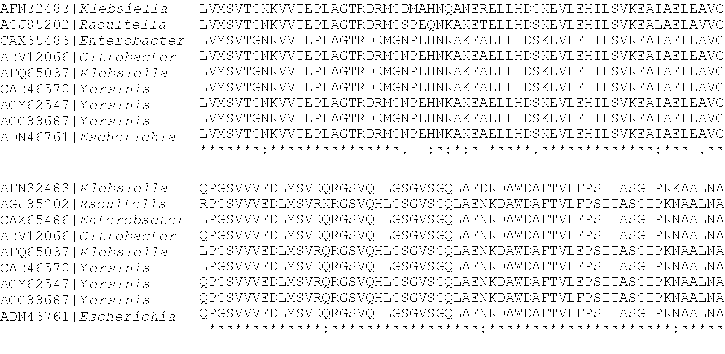

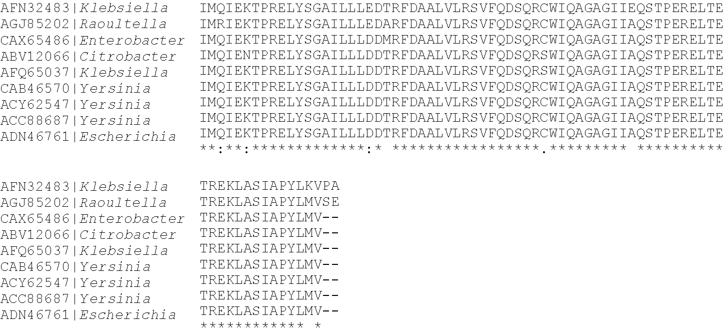

Another example of a salicylic acid synthase is Mbt1 from Mycobacterium tuberculosis (SEQ ID NO: 10). Also provided are polynucleotide sequences that encode salicylic acid synthases, for example, polynucleotide sequences that encode salicylic acid synthases from Klebsiella oxytoca E718 (SEQ ID NO: 11), Raoultella ornithinolytica B6 (SEQ ID NO: 12), Enterobacter hormaechei (SEQ ID NO: 13), Citrobacter koseri ATCC BAA-895 (SEQ ID NO: 14), Klebsiella pneumoniae subsp. pneumoniae 1084 (SEQ ID NO: 15), Yersinia enterocolitica (SEQ ID NO: 16), Yersinia pestis D182038 (SEQ ID NO: 17), Yersinia pseudotuberculosis PB1/+ (SEQ ID NO: 18) and Escherichia coli ABU 83972 (SEQ ID NO: 19).

Modified, active forms of a salicylic acid synthase are also provided herein. These can include truncations, mutations, substitutions, and deletions. By way of example, conservative amino acid substitutions can be made in one or more of the amino acid residues of the polypeptide to generate an active version thereof. One of skill in the art would know that a conservative substitution is the replacement of one amino acid residue with another that is biologically and/or chemically similar. The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

Also provided are nucleic acids and polypeptides having at least, about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity to the wild type sequences set forth herein, wherein the polypeptide encoded by the nucleic acid or the polypeptide has salicylic acid synthase activity. Those of skill in the art readily understand how to determine the identity of two polypeptides or nucleic acids. For example, the identity can be calculated after aligning the two sequences so that the identity is at its highest level.

Another way of calculating identity can be performed by published algorithms. Optimal alignment of sequences for comparison can be conducted using the algorithm of Smith and Waterman Adv. Appl. Math. 2: 482 (1981), by the alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (http://www.ncbi.nlm.nih.gov/blast/bl2seq/bl2.html), or by inspection.

The same types of identity can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 that are herein incorporated by this reference for at least material related to nucleic acid alignment. It is understood that any of the methods typically can be used and that, in certain instances, the results of these various methods may differ, but the skilled artisan understands if identity is found with at least one of these methods, the sequences would be said to have the stated identity.

For example, as used herein, a sequence recited as having a particular percent identity to another sequence refers to sequences that have the recited identity as calculated by any one or more of the calculation methods described above. For example, a first sequence has 80 percent identity, as defined herein, to a second sequence if the first sequence is calculated to have 80 percent identity to the second sequence using the Zuker calculation method even if the first sequence does not have 80 percent identity to the second sequence as calculated by any of the other calculation methods. As yet another example, a first sequence has 80 percent identity, as defined herein, to a second sequence if the first sequence is calculated to have 80 percent identity to the second sequence using each of calculation methods (although, in practice, the different calculation methods will often result in different calculated identity percentages).

### Nucleic Acid Constructs

Provided herein is heterologous expression cassette, wherein the expression cassette comprises a promoter operably linked to a polynucleotide, wherein the polynucleotide encodes a fusion polypeptide comprising a chloroplast targeting peptide and a single enzyme that catalyzes the conversion of chorismate to salicylic acid.

As used throughout, an expression cassette refers to a nucleic acid construct, which when introduced into a host cell (for example, a plant cell), results in transcription and/or translation of a RNA or polypeptide, respectively.

As used throughout, a heterologous expression cassette is a nucleic acid construct comprising sequences that are not operatively linked or are not contiguous to each other in nature. Further, a polynucleotide sequence is heterologous to an organism, for example a plant, or to a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety).

As used herein, a nucleic acid or polynucleotide refers to a deoxyribonucleotide or ribonucleotide in either single- or double-stranded form. The nucleic acid can be a cDNA. The term encompasses nucleic acids containing known analogues of natural nucleotides which have similar or improved binding properties, for the purposes desired, as the reference nucleic acid. The term also includes nucleic acids which are metabolized in a manner similar to naturally occurring nucleotides or at rates that are improved for the purposes desired. The term also encompasses nucleic-acid-like structures with synthetic backbones. DNA backbone analogues include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described in WO 97/03211; WO 96/39154; Mata (1997) Toxicol. Appl. Pharmacol. 144:189-197. Other synthetic backbones encompassed by the term include methyl-phosphonate linkages or alternating methylphosphonate and phosphodiester linkages (Strauss-Soukup (1997) Biochemistry 36: 8692-8698), and benzylphosphonate linkages (Samstag (1996) Antisense Nucleic Acid Drug Dev 6: 153-156).

The nucleic acid constructs described herein can comprise suitable vector backbones including, for example, those routinely used in the art such as plasmids, artificial chromosomes, BACs, YACs, or PACs. Numerous vectors and expression systems are commercially available from such corporations as Novagen (Madison, Wis.), Clontech (Palo Alto, Calif.), Stratagene (La Jolla, Calif.), Invitrogen/Life Technologies (Carlsbad, Calif.) and public repositories or stock centers, such as the Arabidopsis Biological Resource Center (ABRC) at the Ohio State University. In addition to one or more promoters, the vector can comprise other regulatory regions including, but not limited to enhancer sequences, response elements, protein recognition sites, inducible elements, protein binding sequences, 5′ and 3′ untranslated regions (UTRs), transcriptional start sites, termination sequences, polyadenylation sequences, and introns. The vector can further comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosulfuron or Basta.

Numerous promoters can be used in the constructs described herein. A promoter is a region or a sequence located upstream and/or downstream from the start of transcription which is involved in recognition and binding of RNA polymerase and other proteins to initiate transcription. A plant promoter is a promoter capable of initiating transcription in plant cells. A plant promoter can be, but does not have to be, a nucleic acid sequence originally isolated from a plant.

A promoter, or an active fragment thereof, can be employed which will direct expression of a nucleic acid encoding a fusion polypeptide, in all transformed cells or tissues, e.g., as those of a regenerated plant. Such promoters are referred to herein as “constitutive” promoters and are active under most environmental conditions and states of development or cell differentiation. Examples of constitutive promoters include those from viruses which infect plants, such as the cauliflower mosaic virus (CaMV) 35S transcription initiation region (see, e.g., Dagless Arch. Virol. 142:183-191 (1997)); the 1′- or 2′-promoter derived from T-DNA of Agrobacterium tumefaciens (see, e.g., Mengiste supra (1997); O'Grady Plant Mol. Biol. 29:99-108) (1995)); the promoter of the tobacco mosaic virus; the promoter of Figwort mosaic virus (see, e.g., Maiti Transgenic Res. 6:143-156) (1997)); actin promoters, such as the Arabidopsis actin gene promoter (see, e.g., Huang Plant Mol. Biol. 33:125-139 (1997)); alcohol dehydrogenase (Adh) gene promoters (see, e.g., Millar Plant Mol. Biol. 31:897-904 (1996)); ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBankNo. X15596, Martinez et al. J. Mol. Biol 208:551-565 (1989)), Gpc2 from maize (GenBankNo. U45855, Manjunath et al., Plant Mol. Biol. 33:97-112 (1997)), other transcription initiation regions from various plant genes known to those of skill. See also Holtorf (1995) “Comparison of different constitutive and inducible promoters for the overexpression of transgenes in Arabidopsis thaliana,” Plant Mol. Biol. 29:637-646.

Alternatively, a plant promoter can direct expression of the nucleic acids under the influence of changing environmental conditions or developmental conditions. Examples of environmental conditions that can effect transcription by inducible promoters include anaerobic conditions, elevated temperature, drought, or the presence of light. Promoters that can be induced upon infection by a pathogen are also contemplated. Examples of developmental conditions that could effect transcription by inducible promoters include senescence and embryogenesis. Such promoters are referred to herein as inducible promoters.

Other examples of developmental conditions include cell aging, and embryogenesis. For example, contemplated herein are the senescence inducible promoter of Arabidopsis, SAG 12, (Gan and Amasino, Science, 270:1986-1988 (1995)) and the embryogenesis related promoters of LEC1 (Lotan et al., Cell, 93:1195-205 (1998)), LEC2 (Stone et al., Proc. Natl. Acad. of Sci., 98:11806-11811 (2001)), FUS3 (Luerssen, Plant J. 15:755-764 (1998)), AtSERK1 (Hecht et al. Plant Physiol 127:803-816 (2001)), AGL15 (Heck et al. Plant Cell 7:1271-1282 (1995)), and BBM (BABYBOOM). Other inducible promoters include, e.g., the drought-inducible promoter of maize (Busk supra (1997)) and the cold, drought, and high salt inducible promoter from potato (Kirch Plant Mol. Biol. 33:897-909 (1997)).

Alternatively, plant promoters which are inducible upon exposure to plant hormones, such as auxins or cytokinins, are used to express the nucleic acids described herein For example, auxin-response elements E1 promoter fragment (AuxREs) in the soybean (Glycine max L.) (Liu Plant Physiol. 115:397-407 (1997)); the auxin-responsive Arabidopsis GST6 promoter (also responsive to salicylic acid and hydrogen peroxide) (Chen Plant J. 10:955-966 (1996)); the auxin-inducible parC promoter from tobacco (Sakai 37:906-913 (1996)); a plant biotin response element (Streit Mol. Plant Microbe Interact. 10:933-937 (1997)); and, the promoter responsive to the stress hormone abscisic acid (Sheen Science 274:1900-1902 (1996)) can be used. Also provided are the cytokinin inducible promoters of ARRS (Brandstatter and Kieber, Plant Cell, 10:1009-1019 (1998)), ARR6 (Brandstatter and Kieber, Plant Cell, 10:1009-1019 (1998)), ARR2 (Hwang and Sheen, Nature, 413:383-389 (2001)), the ethylene responsive promoter of ERF1 (Solano et al., Genes Dev. 12:3703-3714 (1998)), and the β-estradiol inducible promoter of XVE (Zuo et al., Plant J, 24:265-273 (2000)).

Plant promoters which are inducible upon exposure to chemical reagents which can be applied to the plant, such as herbicides or antibiotics, are also used to express the nucleic acids set forth herein. For example, the maize In2-2 promoter, activated by benzenesulfonamide herbicide safeners, can be used (De Veylder Plant Cell Physiol. 38:568-577 (1997)) as well as the promoter of the glucocorticoid receptor protein fusion inducible by dexamethasone application (Aoyama, Plant J., 11:605-612 (1997)). The coding sequence of the described nucleic acids can also be under the control of, e.g., a tetracycline-inducible promoter, e.g., as described with transgenic tobacco plants containing the Avena sativa L. (oat) arginine decarboxylase gene (Masgrau Plant J 11:465-473 (1997)); or, a salicylic acid-responsive element (Stange Plant J. 11:1315-1324 (1997)).

Tissue specific promoters can also be used. Examples of tissue-specific promoters under developmental control include promoters that initiate transcription only (or primarily only) in certain tissues, such as vegetative tissues, e.g., roots, leaves or stems, or reproductive tissues, such as fruit, ovules, seeds, pollen, pistils, flowers, or any embryonic tissue.

A variety of promoters specifically active in vegetative tissues, such as leaves, stems, roots and tubers, can also be used to express the nucleic acids used in the methods of the invention. For example, promoters controlling patatin, the major storage protein of the potato tuber, can be used, e.g., Kim Plant Mol. Biol. 26:603-615 (1994); Martin Plant J. 11:53-62 (1997). The ORF13 promoter from Agrobacterium rhizogenes which exhibits high activity in roots can also be used (Hansen Mol. Gen. Genet. 254:337-343 (1997)). Other useful vegetative tissue-specific promoters include: the tarin promoter of the gene encoding a globulin from a major taro (Colocasia esculenta L. Schott) corm protein family, tarin (Bezerra Plant Mol. Biol. 28:137-144 (1995)); the curculin promoter active during taro corm development (de Castro Plant Cell 4:1549-1559 (1992)) and the promoter for the tobacco root-specific gene TobRB7, whose expression is localized to root meristem and immature central cylinder regions (Yamamoto Plant Cell 3:371-382 (1991)).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier FEBS Lett. 415:91-95 (1997)). A ribulose bisphosphate carboxylase promoters expressed almost exclusively in mesophyll cells in leaf blades and leaf sheaths at high levels, described by Matsuoka Plant J. 6:311-319 (1994), can be used. Another leaf-specific promoter is the light harvesting chlorophyll a/b binding protein gene promoter, see, e.g., Shiina Plant Physiol. 115:477-483 (1997); Casal Plant Physiol. 116:1533-1538 (1998). The Arabidopsis thaliana myb-related gene promoter (Atmyb5) described by Li FEBS Lett. 379:117-121 (1996), is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16-cell stage of embryo development and persists beyond the heart stage. A leaf promoter identified in maize by Busk Plant J. 11:1285-1295 (1997), can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, described by Di Laurenzio Cell 86:423-433 (1996) and Long Nature 379:66-69 (1996), can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto Plant Cell. 7:517-527 (1995)). Also useful are kn1-related genes from maize and other species which show meristem-specific expression, see, e.g., Granger Plant Mol. Biol. 31:373-378 (1996); Kerstetter Plant Cell 6:1877-1887 (1994); Hake Philos. Trans. R. Soc. Lond. B. Biol. Sci. 350:45-51 (1995). For example, the Arabidopsis thaliana KNAT1 or KNAT2 promoters. In the shoot apex, KNAT1 transcript is localized primarily to the shoot apical meristem; the expression of KNAT1 in the shoot meristem decreases during the floral transition and is restricted to the cortex of the inflorescence stem (see, e.g., Lincoln Plant Cell 6:1859-1876 (1994)).

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

Any of the nucleic acids described herein can be expressed through a transposable element. This allows for constitutive, yet periodic and infrequent expression of the constitutively active polypeptide. The invention also provides for use of tissue-specific promoters derived from viruses which can include, e.g., the tobamovirus subgenomic promoter (Kumagai Proc. Natl. Acad. Sci. USA 92:1679-1683 (1995)) the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer Plant Mol. Biol. 31:1129-1139 (1996)).

Any of the polynucleotides described herein can be operably linked to a chloroplast targeting or transient sequence that directs expression to the chloroplast. For example, a chloroplast targeting sequence can direct expression of a polynucleotide encoding a single enzyme catalyzing conversion of chorismate to salicylic acid to the chloroplast. Examples of these sequences include, but are not limited to the choloroplast transient sequence from ferredoxin (FD), ribulose bisphosphate carboxylase (Rubisco) small subunit (Wong et al. Plant Mol. Biol. 20:81-93 (1992)), Rubisco activase (Hyunjong et al. J. Exp. Bot. 57: 161-169 (2006)), or granule-bound starch synthase (Di Fiore et al. Plant Physiology 129:1160-1169 (2002)), or other synthetic plastid transient peptides (Bruce, Biochim. Biophys. Acta 1541:2-21 (2001)). Synthetic versions of these targeting sequences are also provided. A non-limiting example of a polynucleotide sequence encoding a targeting sequence from Arabidopsis is provided herein as SEQ ID NO: 20. Non-limiting examples of polynucleotide sequences encoding a fusion polypeptide comprising a chloroplast targeting peptide and a single enzyme that catalyzes the conversion of chorismate to salicylic acid are provided herein as SEQ ID NO: 21 and SEQ ID NO: 22. SEQ ID NO: 21 encodes a chloroplast targeting peptide from Arabadopsis and a salicylic acid synthase (Irp9) from Yersinia enterocolitica. SEQ ID NO: 22 is a polynucleotide sequence comprising a 35S promoter, a polynucleotide sequence encoding a plastid targeting sequence from Arabidopsis FD2, a polynucleotide sequence encoding Irp9 and an NOS terminator, as described in the Examples.

### Production of Transgenic Plants

As discussed above, the plants described herein comprise a heterologous expression cassette, wherein the expression cassette comprises a promoter operably linked to a polynucleotide, wherein the polynucleotide encodes a fusion polypeptide comprising a chloroplast targeting peptide and a single enzyme that catalyzes the conversion of chorismate to salicylic acid. Accordingly, transgenic plants comprising the heterologous expression cassette are provided herein. Methods of producing these transgenic plants are also provided.

Provided herein is a method of producing any of the plants described herein comprising a) transforming a plant cell or a plant seed with a heterologous expression cassette, wherein the expression cassette comprises a promoter operably linked to a polynucleotide, wherein the polynucleotide encodes a fusion polypeptide comprising a chloroplast targeting peptide and a single enzyme that catalyzes the conversion of chorismate to salicylic acid; and b) regenerating a transgenic plant from said transformed plant cell or plant seed.

Nucleic acid constructs, for example, DNA constructs, described herein can be introduced into the genome of the desired plant host by a variety of conventional techniques. For example, the DNA constructs can be introduced directly into the genomic DNA of the plant cell using techniques such as electroporation and microinjection of plant cell protoplasts, or the DNA constructs can be introduced directly to plant tissue using biolistic methods, such as DNA particle bombardment. Alternatively, the DNA constructs can be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski et al. Embo J. 3:2717-2722 (1984). Electroporation techniques are described in Fromm et al. Proc. Natl. Acad. Sci. USA 82:5824 (1985). Biolistic transformation techniques are described in Klein et al. Nature 327:70-73 (1987).

Agrobacterium tumefaciens-mediated transformation techniques, including disarming and use of binary vectors, are well described in the scientific literature. See, for example Horsch et al., Science 233:496-498 (1984), and Fraley et al. Proc. Natl. Acad. Sci. USA 80:4803 (1983).

Transformed plant cells which are derived by any of the above transformation techniques can be cultured to regenerate a whole plant which possesses the transformed genotype and thus the desired phenotype such as increased stress tolerance compared to a control plant that was not transformed or transformed with an empty vector. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987). Also provided herein are the progeny of crosses (controlled or naturally occurring) derived from the transgenic plants described herein, wherein the progeny is obtained without additional transformation or tissue culture propagation.

The nucleic acids and encoded polypeptides described can be used to confer increased stress tolerance on essentially any plant, including crops. Thus, the invention has use over a broad range of plants, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pinus, Pisum, Populus, Pyrus, Prunus, Raphanus, Secale, Senecio, Sinapis, Solanum, Sorghum, Trigonella, Triticum, Vitis, Vigna, and, Zea. Examples of crops include, but are not limited to, alfalfa, canola, corn, cotton, papaya, potato, rice, soybeans, squash, sugar beet, sugarcane, sweet peppers, tomatoes and wheat.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations may not be explicitly disclosed, each is specifically contemplated and described herein. For example, if a method is disclosed and discussed and a number of modifications that can be made to a number of compositions included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

A number of embodiments have been described. Nevertheless, it will be understood that various modifications may be made. Accordingly, other embodiments are within the scope of the following claims.

### EXAMPLES

### Example 1

As shown herein, the bacterial SA biosynthesis and degradation pathways were engineered into Populus to generate metabolite and gene correlation networks for investigating SA function. SA increases by two to three orders of magnitude elicited strong oxidative stress responses in Populus without compromising growth. Network analysis identified metabolite and gene clusters associated with changing carbon inputs, phenylpropanoid homeostasis and redox regulation during Populus responses to elevated SA.

### Methods

### Generation of Transgenic Populus

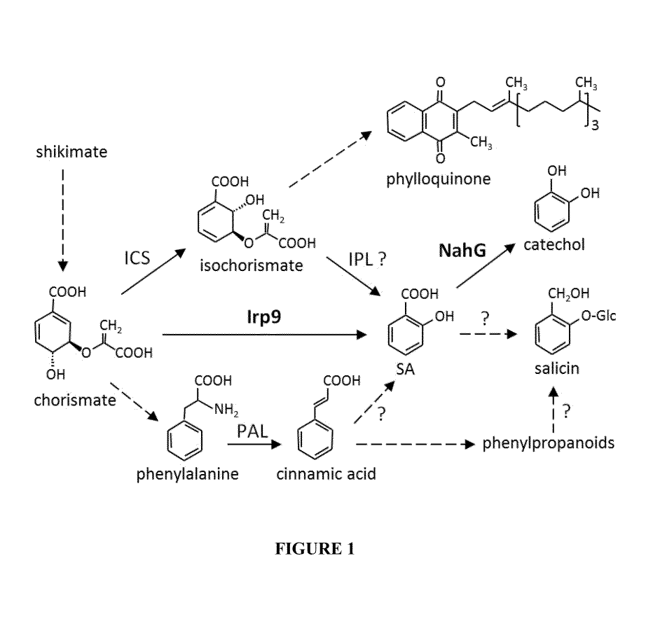

The Yersinia enterocolitica Irp9 gene was PCR amplified from plasmid pTIrp9 (Pelludat et al. J. Bacteriol. 185, 5648-5653 (2003)) with primers that introduced 5′-Sal I or Bgl II and 3′-Nhe I sites (Table 1), TOPO-TA cloned into pCR2.1 (Invitrogen, Grand Island, N.Y.) and sequence-verified to produce pCR-Irp9a and pCR-Irp9b, respectively. FIG. 1 shows the reaction catalyzed by Irp9. The chloroplast transient peptide sequence from Arabidopsis FD2 gene (At1g60950) was PCR amplified with 5′-Bgl II and 3′-Sal I sites and inserted in-frame upstream of pCR-Irp9a to generate pCR-FD-Irp9, and sequence-confirmed. The Irp9 or FD-Irp9 fragments were then subcloned into pCambia1302 at Bgl II and Nhe I sites downstream of the 35S promoter to generate the respective binary constructs. The NahG construct in pCIB200 (Gaffney et al., Science 261: 754-756 (1993)) was provided by Syngenta Biotechnology Inc. (Research Triangle Park, NC). Transformation of P. tremula×alba clone 717-1B4 was carried out as described (Meilan and Ma, In Methods in Mol. Biol. 34: Agrobacterium Protocols, K. Wang ed. (Humana Press), pp. 143-151 (2006)). Primary transformants were transplanted to soil and maintained in a greenhouse for initial characterization. Selected transgenic lines along with WT were vegetatively propagated by rooted cuttings as described (Frost et al., 2012) for subsequent experiments.

TABLE 1
Primers
Primers used in this study.

| | Gene model or | | | |
|---|---|---|---|---|
| Gene | accession no. | Forward primer (5′ to 3′) | Reverse primer (5′ to 3′) | Purpose |
| Irp9 | CAB46570 | GTCGACATGAAAATCAGTGAATTTC | GCTAGCCTACACCATTAAATAGGGC | cloning |
| | | (SEQ ID NO: 23) or | (SEQ ID NO: 25) | |
| | | AGATCTATGAAAATCAGTGAATTTC | AGGGCGCAATGCTCGCTAATTTCT | qRT-PCR |
| | | (SEQ ID NO: 24) | (SEQ ID NO: 27) | |
| | | ATGCGTTTACCGTGCTGTTTCCGT | | |
| | | (SEQ ID NO: 26) | | |
| FD | At1g60950 | AGATCTAAAATGGCTTCCACTGCTCTC | GTCGACGACCTTGTATGTAGCCATGGC | cloning |
| | | (SEQ ID NO: 28) | (SEQ ID NO: 29) | |
| NahG | M60055 | AACCTCGCCGAGCTGCTTGA | AGGTCAGTGTCGAGGTCGTGGT | qRT-PCR |
| | | (SEQ ID NO: 30) | (SEQ ID NO: 31) | |
| NRX1 | POPTR_0008s17570, | TGCCTTGGTTAGCCCTTCCATTTG | TGTCARGTGCWTCCGAGCTTCCTT | qRT-PCR |
| | POPTR_0010s06930- | (SEQ ID NO: 32) | (SEQ ID NO: 33) | |
| | POPTR_0010s07010 | | | |
| WRKY | POPTR_0006s27950 | ACCATGCTAGCCATTCTCAACCTG | TTAGCACTGTCAACGTGCATTCCA | qRT-PCR |
| | | (SEQ ID NO: 34) | (SEQ ID NO: 35) | |
| WRKY | POPTR_0016s14490 | GTGATCACGCTTCRAACAAGCCAA | TACCATCCATGTCCARACTGTGTGAG | qRT-PCR |
| | | (SEQ ID NO: 36) | (SEQ ID NO: 37) | |
| RLK | POPTR_0012s01760 | ACGCGCAAAYSGCAAAGAAGCTGA | TCTGTTTCARWGATCACYTCCAACGC | qRT-PCR |
| | | (SEQ ID NO: 38) | (SEQ ID NO: 39) | |
| RLK | POPTR_0017s09520 | TGGAGGAAGGAAGAACGTCGATGA | TGCRCTTGTTYTCTTAGGGACAGAGG | qRT-PCR |
| | | (SEQ ID NO: 40) | (SEQ ID NO: 41) | |
| EF1-6 | POPTR_0001s23190 | GACCTKGTATCAGTGGATTCCCTC | GAACAGAGGCACAAGATTACCAGG | qRT-PCR |
| | | (SEQ ID NO: 42) | (SEQ ID NO: 43) | |
| ARP | POPTR_0017s08430 | ACTGTGAGGAGATGCAGAAACGCA | GCTGTGTCACGGGCATTCAATGYT | qRT-PCR |
| | | (SEQ ID NO: 44) | (SEQ ID NO: 45) | |
| TAF | POPTR_0001s37010 | CGTGCAGCTGGTCTCTRTATGTAT | ACTGACACACTGGAAGCTCCAACA | qRT-PCR |
| | | (SEQ ID NO: 46) | (SEQ ID NO: 47) | |

### Heat Experiments

Two heat experiments were conducted. One experiment was designed to monitor photosynthetic responses to temperature variations in a growth chamber. Vegetatively propagated WT and two transgenic lines each of FD-Irp9 (F52 and F55) and NahG (N24 and N31) plants were grown under NT (27(717° C., day/night) until they reached ˜140 cm in height. After initial (pre-stress) photosynthesis measurements, the chamber setting was changed to HT (35°/25° C., day/night) for 10 days before returning to NT for recovery. Photosynthetic responses were measured three times during the 10-day HT period and once after 1-week recovery. Two leaves (LPI-5 and LPI-10) were measured independently on each plant using a Licor LI-6400XS (LiCor) at a saturating light intensity of 1500 μmol/m²/s as described (Frost et al., 2012). To test the effects of HT treatment, genotype and their interaction on photosynthesis, data were analyzed by repeated measures ANOVA, using a linear mixed effect model with the lme function in R. Significant genotypic differences were further analyzed by pairwise comparisons between WT and each transgenic line.

The other heat experiment was designed for comparative analyses of plant growth, electrolyte leakage, gene expression and metabolite response under different temperature regimes. WT and 2-3 lines each of the Irp9 (I6 and I8), FD-Irp9 (F10, F52 and F55) and NahG (N24, N31 and N51) transgenics were used. Vegetatively propagated plants were randomly assigned to two identical growth chambers and grown to a height of −120 cm under NT. At that time, plants in one chamber were subjected to HT, while the other chamber was maintained at NT. One week after the HT treatment commenced, leaf discs from LPI-6 were collected for electrolyte leakage assays and LPI-1 and LPL-5 were snap-frozen in liquid nitrogen for gene expression and metabolite analyses. After sampling, the treatment chamber was reset to NT for recovery, and samples were collected again one week later. All sampling was conducted in the light during mid-day and under the specified chamber temperature (NT or HT).

### Plant Growth

Plant height growth was monitored n the two-chamber heat experiment from September to November of 2010. Because initial plant sizes varied, and because height growth was approximately linear during the monitoring period for all plants, height increment per unit time was used for statistical analysis by repeated measures ANOVA. Height and diameter growth were also monitored using a separate cohort of plants in a greenhouse experiment during June 2011. Evaporative cooling was used to maintain greenhouse temperatures ˜5° C. below daytime ambient temperatures, which ranged 33-37° C. maximum during this time. Genotypic differences of growth rate were tested by repeated measures ANOVA.

### Electrolyte Leakage Analysis

Two 6-mm leaf discs (LPI-6) per plant were collected into 5 ml of ddH₂O during tissue harvesting of the heat experiment and kept on ice in the dark for up to 4 hours. Upon returning to the lab, the samples were incubated at ambient temperature under light for 6-7 hours with gentle shaking. The incubation time was chosen based on preliminary testing that the electrolyte leakage reached a plateau by this time. Conductivity was measured using a Traceable™ conductivity meter. The samples were then boiled for 30 min and cooled to room temperature overnight before measurements were taken again. Blank (ddH₂O)-corrected conductivity values were used for statistical analysis by Student's t-test. To evaluate temperature-dependent electrolyte leakage, leaf discs from LPI-5 of greenhouse-grown WT and F10 plants were randomly distributed into three tubes (5 discs per tube with 10 ml of ddH₂O). The tubes were incubated at 25° C., 37° C. and 50° C., and electroconductivity was monitored for four hours. The samples were then boiled for 30 min to release total electrolytes. Measurements for the 37° C., 50° C. and boiled samples were taken after cooling to room temperature. Blank-corrected conductivity values were used for statistical analysis by repeated measures ANOVA.

### Metabolite Profiling and Data Analysis

For initial transgenic plant screening, freeze-dried leaf powder (5 mg) was extracted in 500 μl of methanol containing ¹³C₆-cinnamic acid, D₅-benzoic acid and resorcinol as internal standards by sonication in ice water for 5 min. Following centrifugation, the extracts were stored at −80° C. until HPLC analysis. For the heat experiments, freeze-dried leaf powder (10 mg) was extracted twice with 670 μl of methanol:water:chloroform (46:30:24) containing ¹³C₆-cinnamic acid, D₅-benzoic acid, resorcinol, 2-methoxybenzoic acid and adonitol as internal standards. Following vortexing and incubation at 70° C. for 5 min (first extraction) and sonication in ice water for 15 min (second extraction), the aqueous phase from both extractions were combined. A fraction of the extract (700 μl) was evaporated to dryness in a CentriVap (Labconco, Kansas City, Mo.), and the rest saved for HPLC analysis. Dried aliquots were resuspended in 40% methanol and further partitioned into relatively polar and non-polar fractions using Advanta resin (Applied Separations, Allentown, Pa.) for GC-MS analysis as described (Jeong et al., Plant Physiology 136: 3364-3375 (2004)); Frost et al. PLoS ONE 7: e44467 (2012)). Mass spectral data were processed by AnalyzerPro (Spectral Works, Runcorn, UK) for deconvolution and matching against the NIST08 (Babushok et al., Journal of Chromatography A 1157: 414-421 (2007)), FiehnLib (Kind et al., Analytical Chem. 81: 10038-10048 (2009)); Agilent) and in-house authentic standard mass spectral libraries. The output files were then processed by a custom web-based pipeline, MetaLab, for compound matching between samples based on retention index and mass spectral similarity, followed by manual curation. Original datasets available at the MetaLab website (www.aspenDB.uga.edu) under Analysis IDs 70, 71, 128 and 353.

Phenolic compounds were analyzed on an Agilent 1200 HPLC equipped with a diode array detector (DAD) and a 6220 accurate mass time-of-flight mass spectrometer with dual electrospray ionization (ESI), using a ZORBAX Rapid Resolution Eclipse XDB-C18 column (4.6×50 mm, 1.8 μm, Agilent) and mobile phase solvents water:acetonitrile:formic acid=97:3:0.1 (A) and 3:97:0.1 (B). The elution gradient was 3% B from 0-1 min, linear gradient to 17% B over 2 min, isocratic at 17% B for 2 min, linear gradient to 60% B over 4 min, and then to 98% B over 2 min, at a flow rate of 1 ml/min. DAD detection was set at 260, 270, 280, 310, and 350 nm, and MS acquisition at m/z 100-1500 in negative ESI mode with the following parameters: gas temperature 350° C., drying gas flow 13 l/min, nebulizer pressure 60 psig, capillary voltage 3500 V and fragmentor voltage 125 V. Data were processed by MassHunter Qualitative Analysis, Mass Profiler and MassHunter Quantitative Analysis software suite (Agilent), followed by manual curation. Metabolite identity was confirmed by authentic standards when possible, or by searching the predicted m/z and molecular formula against the KNApSAcK database (Mochamad Afendi et al., 2011). SAG and GAG peaks were isolated using a fraction collector and treated with β-glucosidase. Aglycone identity was confirmed by comparison with authentic standards.

Relative abundance was determined as peak area of each metabolite divided by that of the internal standard (2-methoxybenzoic acid for GC polar, adonitol for GC non-polar, and D₅-benzoic acid for HPLC metabolites), followed by a correction for differences in tissue dry weight. Metabolites that were significantly changed by HT or transgenic manipulation (p value≦0.1 by Student's t) were subjected to clustering analysis. The data were normalized by compound using the Z-score method, and clustering was performed using R function Heatmap.2 with the Pearson distance metrics and the average linkage method. To visualize metabolite correlations in a network context, the Pearson correlation coefficient (PCC) was calculated for all metabolite pairs. Significant associations (absolute PCC≧0.5, p≦0.05) were visualized in Cytoscape v2.8.2 (Smoot et al., 2011).

### Microarray Design, Hybridization and Data Analysis

A new Agilent Poplar array (v2) was designed based on the JGI Populus genome release v2.2 for the 8×60K array platform. The CDS region was targeted for probe design, since the 3′-UTR is less conserved, sometimes with indels, among Populus species (see Tsai et al., Poplar genome microarrays. In Genetics, Genomics and Breeding of Poplars, C. P. Joshi, S. P. DiFazio and C. Kole eds. (Enfiled, NH: Science Publishers) pp. 112-127 (2011b)). CDS sequences were first blasted against one another to remove highly similar sequences from different gene models. A series of probes was then designed using eArray (Agilent), and custom Perl scripts were used to evaluate probe specificity. A total of 43,070 probes were selected for the 40,330 predicted v2.2 gene models, more than 96% of which (38,770) were represented by specific probes (no other matches with ≧90% identity). 12,080 probes designed for mRNA sequences that had poor or no matches in the v2.2 genome, in addition to 1,160 probes for microRNAs and 295 probes for mitochondrial or chloroplastic gene models were also included. Probes corresponding to the Irp9 and NahG transgenes, as well as several reporter or selectable marker genes commonly used in transgenic research were also included.

RNA for microarray analysis was extracted from LPI-5 using the CTAB method (Tsai et al., 2011a), and treated with the Turbo DNA-free kit (Ambion (Grand Island, N.Y.)) to remove genomic DNA. Two biological replicates were included for each genotype. cRNA target labeling was performed as described (Syed and Threadgill, 2006) using the One-color Quick Amp Labeling Kit (Agilent (Santa Clara, Calif.)). Array hybridization and washing were carried out with Agilent reagents and instructions. Arrays were scanned at 3 μm resolution and images processed using Feature Extraction v10.5.1.1 (Agilent). Intensity data were normalized by 75^{th} percentile-shift using GeneSpring GX11. Probes with low expression values (≦300 in all samples) were excluded, leaving 21,313 probes for further analysis.

DE was assessed using Limma (Smyth, 2005) and SLIM (Wang et al., 2011) packages, unless otherwise specified. SLIM uses p values obtained from Limma and applies a sliding linear model to estimate π₀ for a robust control of false discovery rate in multiple hypothesis testing. The significance threshold was fold-change≧2 and SLIM pₘₐₓ=0.05. GO enrichment analysis was conducted using R package topGO (Alexa et al., 2006), with GO annotation obtained from agriGO (Du et al., Nucleic Acids Research 38: W64-W70 (2010)) and Arabidopsis GO Slim (TAIR). Significance of enrichment was determined by Fisher's exact test and the negative log 10 transformed p values were used for visualization in heatmaps. To reduce redundancy, semantic similarity among GO terms was computed using GOSemSim (Yu et al., Bioinformatics 26: 976-978 (2010)), followed by cutting the hierarchical tree of GO categories into clusters. Representative GOs from the clusters, typically those with lower p values and/or higher GO hierarchy levels, were selected for visualization.

### Gene Network Construction and Visualization

Co-expression networks were constructed using the WGCNA (v1.18.1) package in R (Langfelder and Horvath, Bioinformatics 9: 559 (2008)). DE genes were obtained with a less stringent cutoff (ANOVA p≦0.05 and fold-change≧1.5). The normalized abundance of SAG was included in the network analysis. The adjacency matrix was calculated using a power of 10 that satisfied the scale-free topology criterion. The dynamic tree cut method was used to define co-regulation modules from the hierarchical tree based on topological overlap matrix, with the minimum module size set to 80 genes and the minimum height for merging modules set to 0.1. The eigengene value (first principal component) was calculated for each module and used to test the association with metabolite data. Subnetworks were similarly constructed by dividing the data into WT-like (WT, I6 and N31) and high-SA (F10 and F52) groups, using a power of 14 for the adjacency matrix calculation. The networks were visualized using Cytoscape, with node colors corresponding to module colors from WGCNA. Selected genes were verified by qRT-PCR according to established protocols (Tsai et al., 2006) using elongation factor 1β, actin-related protein, and TATA box binding protein associated factor as housekeeping genes (see Table 1 for primers).

### Meta-Analysis of Populus Leaf Microarray Data Sets

Populus leaf microarray data sets derived from oxidative stress experiments were downloaded from GEO, and included Affymetrix (GSE9673, GSE15242, GSE16783, GSE16785, GSE17226, GSE17230, GSE21171, GSE27693, GSE37608) and EST (GSE10873) microarray data. Affymetrix data were processed by MAS 5 (Affymetrix) and grouped in pairs containing stressed samples and their respective controls. The EST array data were normalized by print-tip LOWESS and quantile methods using codes provided by the original authors in GEO (Street et al., 2011). Quality control filtering was performed to remove probes (probe-sets) that were below detection in each pair of samples (n=2-3). DE was assessed by Limma using linear models (Accerbi et al., Methods for isolation of total RNA to recover miRNAs and other small RNAs from diverse species. In Plant MicroRNAs, B. C. Meyers and P. J. Green, eds (Humana Press), pp. 31-50 (2010)) and fold-change of each probe (treatment/control) was calculated for all sample pairs. Probe annotation against Populus genome v2.2 was performed using an in-house pipeline and is available at http://aspendb.uga.edu/downloads. The fold-change values and DE significance (p-values) were then extracted for the 144 driver or 438 hub genes to generate box-and-whisker plots the using R function boxplot. Not all pairs contain all driver or hub genes due to quality-control filtering.

Accession Numbers

The Agilent microarray platform can be found in the NCBI Gene Expression Omnibus (GEO) under accession no. GPL16322, and is also available on the Agilent eArray website under ID 033484 of “Published Designs”. The microarray data from this study can be found in GEO under accession number GSE42511.

### Results

Characterization of Transgenic Poplars with Altered SA Levels

The bi-functional SA synthase gene Irp9 from the pathogenic bacterium Yersinia enterocolitica (Pelludat et al., 2003a) was introduced to Populus tremula×alba (clone 717-1B4) under control of a constitutive (CaMV 35S) promoter, with or without the plastid-targeting sequence from the Arabidopsis ferredoxin (FD) gene.

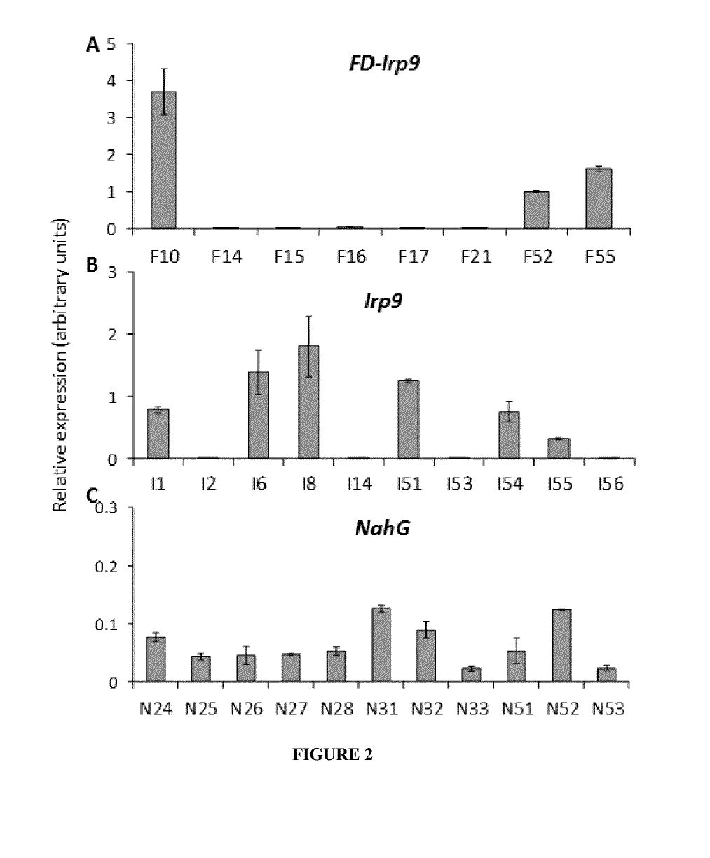

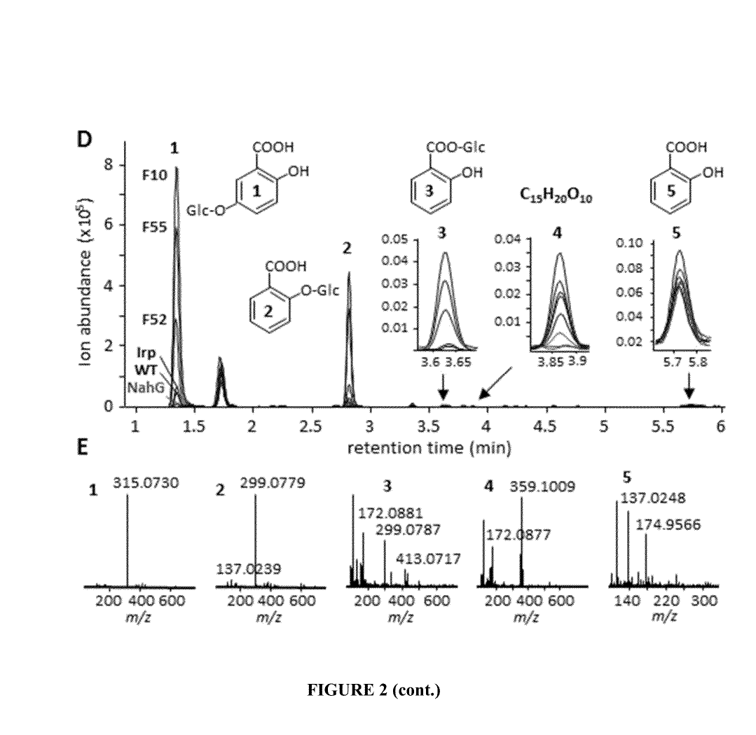

Hereafter, transgenic plants with plastidic or cytosolic targeting of the transgene are referred to as FD-Irp9 and Irp9, respectively. The third group of transgenic plants harbors the Pseudomonas putida NahG gene, also driven by the CaMV 35S promoter (Gaffney et al., 1993). Eight to eleven putative transgenic lines were obtained for each group, and lines with high levels of transgene expression were identified by qRT-PCR (FIG. 2A-C). HPLC-TOF/MS analysis identified a range of SA metabolic phenotypes from leaf methanolic extracts of transgenic plants (see FIG. 2D-E). FD-Irp9 plants exhibited elevated levels of SA-conjugates, including SA-glucoside (SAG), gentisic acid glucoside (GAG) and, to a much smaller extent, SA glucose ester (SGE). The levels of SA metabolites were highest in line F10, followed by F55 and F52, in accordance with the estimated FD-Irp9 transcript abundance (FIG. 2A-C). Cytosolic Irp9 had a minor effect on SA conjugate levels. The three NahG lines examined (N31, N24 and N51) exhibited reduced levels of SA-conjugates, consistent with previous findings.

### Effects of SA Manipulation on Photosynthesis, Stomatal Behavior, Growth and Membrane Integrity Under Different Temperature Regimes

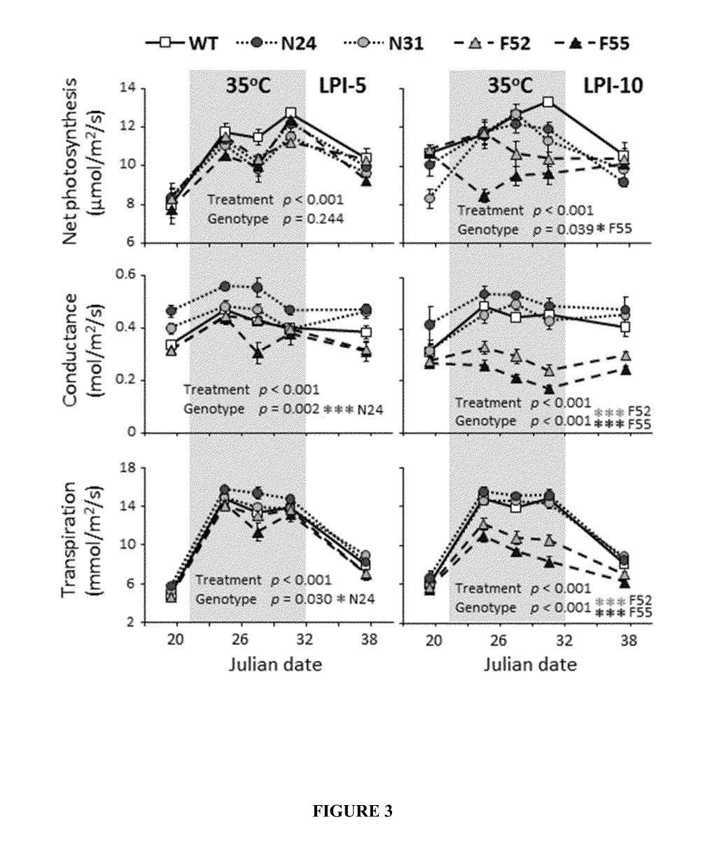

WT and selected transgenic plants were vegetatively propagated to assess the effects of SA perturbation on photosynthesis, growth, metabolite and gene expression responses. Two temperature regimes (27°/17° C. [NT] vs. 35°/25° C. [HT], day/night) were used. Photosynthesis was monitored over a period of 19 days when growth temperatures were varied from NT to HT and then back to NT, using WT and two lines each of the FD-Irp9 (F52 and F55) and NahG (N24 and N31) transgenics. Repeated measures ANOVA revealed significant differences in photosynthetic properties of both young (leaf plastochron index LPI-5) and mature (LPI-10) source leaves over time, reflecting significant treatment effects (FIG. 3). Significant genotypic differences were also detected, and the responses were stronger in mature than young source leaves. Overall, net photosynthesis, stomatal conductance and transpiration rates increased significantly at HT. However, the responses were significantly attenuated in the mature source leaves of FD-Irp9 plants. As a result, FD-Irp9 lines exhibited significantly reduced net photosynthesis, stomatal conductance and transpiration relative to the WT under HT growth (FIG. 3, and Table 2).

TABLE 2
Repeated measures ANOVA of photosynthetic responses presented in FIG. 2.
Significant genotypic effects were further analyzed by pairwise comparison
between wild-type (WT) and individual transgenic lines. Df(num), numerator
degrees of freedom; Df(den), denominator degrees of freedom.

| Leaf | Measurement | Source | Df(num) | Df(den) | F | Pr > F |
|---|---|---|---|---|---|---|
| LPI-10 | Aₘₐₓ | genotype | 4 | 24 | 2.983 | 0.0393 |
| | | treatment | 4 | 96 | 11.813 | <0.0001 |
| | | genotype × treatment | 16 | 96 | 3.180 | 0.0002 |
| | | WT vs. F52 | 1 | 14 | 3.608 | 0.0783 |
| | | WT vs. F55 | 1 | 11 | 9.210 | 0.0114 |
| | | WT vs. N24 | 1 | 12 | 1.490 | 0.2456 |
| | | WT vs. N31 | 1 | 14 | 3.023 | 0.1040 |
| Conductance | | genotype | 4 | 24 | 11.137 | <0.0001 |
| | | treatment | 4 | 96 | 9.477 | <0.0001 |
| | | genotype: treatment | 16 | 96 | 1.614 | 0.0795 |
| | | WT vs. F52 | 1 | 14 | 20.373 | 0.0005 |
| | | WT vs. F55 | 1 | 11 | 24.351 | 0.0004 |
| | | WT vs. N24 | 1 | 12 | 2.402 | 0.1471 |
| | | WT vs. N31 | 1 | 14 | 0.044 | 0.8369 |
| Transpiration | | genotype | 4 | 24 | 14.905 | <0.0001 |
| | | treatment | 4 | 96 | 248.961 | <0.0001 |
| | | genotype: treatment | 16 | 96 | 3.711 | <0.0001 |
| | | WT vs. F52 | 1 | 14 | 25.205 | 0.0002 |
| | | WT vs. F55 | 1 | 11 | 45.022 | <0.0001 |
| | | WT vs. N24 | 1 | 12 | 1.513 | 0.2423 |
| | | WT vs. N31 | 1 | 14 | 0.094 | 0.7643 |
| LPI-5 | Aₘₐₓ | Genotype | 4 | 24 | 1.464 | 0.2442 |
| | | Treatment | 4 | 96 | 37.760 | <.0001 |
| | | Genotype × treatment | 16 | 96 | 0.604 | 0.8738 |
| | | WT vs. F52 | 1 | 14 | 2.625 | 0.1275 |
| | | WT vs. F55 | 1 | 11 | 2.927 | 0.1151 |
| | | WT vs. N24 | 1 | 12 | 1.202 | 0.2944 |
| | | WT vs. N31 | 1 | 14 | 3.594 | 0.0788 |
| | Conductance | genotype | 4 | 24 | 5.818 | 0.0020 |
| | | treatment | 4 | 96 | 18.021 | <0.0001 |
| | | genotype: treatment | 16 | 96 | 1.453 | 0.1343 |
| | | WT vs. F52 | 1 | 14 | 1.300 | 0.2734 |
| | | WT vs. F55 | 1 | 11 | 2.714 | 0.1277 |
| | | WT vs. N24 | 1 | 12 | 19.106 | 0.0009 |
| | | WT vs. N31 | 1 | 14 | 4.537 | 0.0514 |
| Transpiration | | genotype | 4 | 24 | 3.217 | 0.0300 |
| | | treatment | 4 | 96 | 552.006 | <0.0001 |
| | | genotype: treatment | 16 | 96 | 1.131 | 0.3386 |
| | | WT vs. F52 | 1 | 14 | 1.444 | 0.2494 |
| | | WT vs. F55 | 1 | 11 | 2.305 | 0.1571 |
| | | WT vs. N24 | 1 | 12 | 8.721 | 0.0121 |
| | | WT vs. N31 | 1 | 14 | 2.861 | 0.1129 |

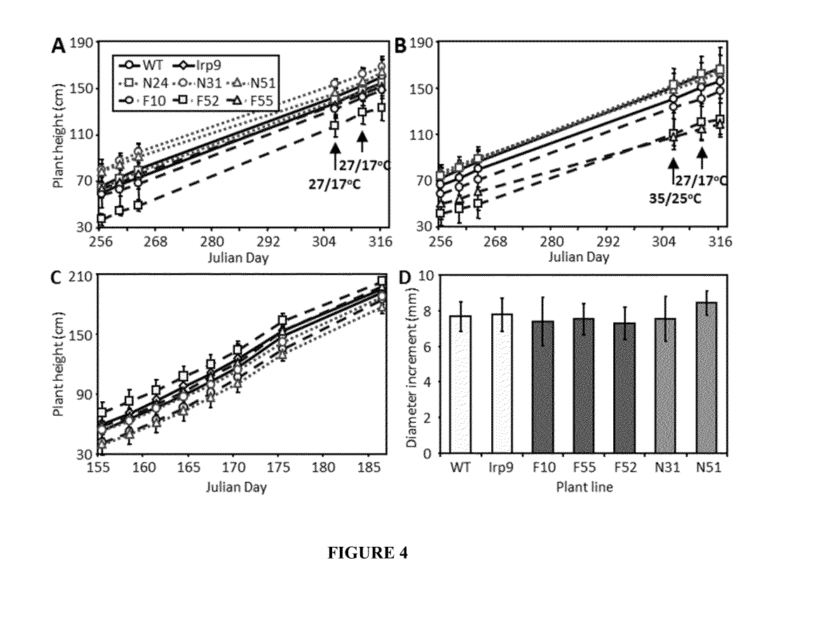

A separate cohort of plants derived from WT and 2-3 lines each of the Irp9 (16 and 18), FD-Irp9 (F10, F52 and F55) and NahG (N24, N31 and N51) transgenic lines was randomly assigned to two growth chambers and maintained under identical (NT) conditions. After 50 days, one chamber was changed to HT for one week before returning to NT for recovery. Plant height growth was monitored over the two-month period. Although initial plant size varied, increment height growth did not differ significantly among genotypes under either temperature regime (FIG. 4A-B). The results were confirmed using another cohort of plants under greenhouse conditions (FIG. 4C-D). The data suggested that unlike in Arabidopsis (Rivas-San Vicente and Plasencia, Journal of Experimental Biology 62: 3321-3338 (2011)), growth was not compromised by constitutive overproduction of SA in Populus.

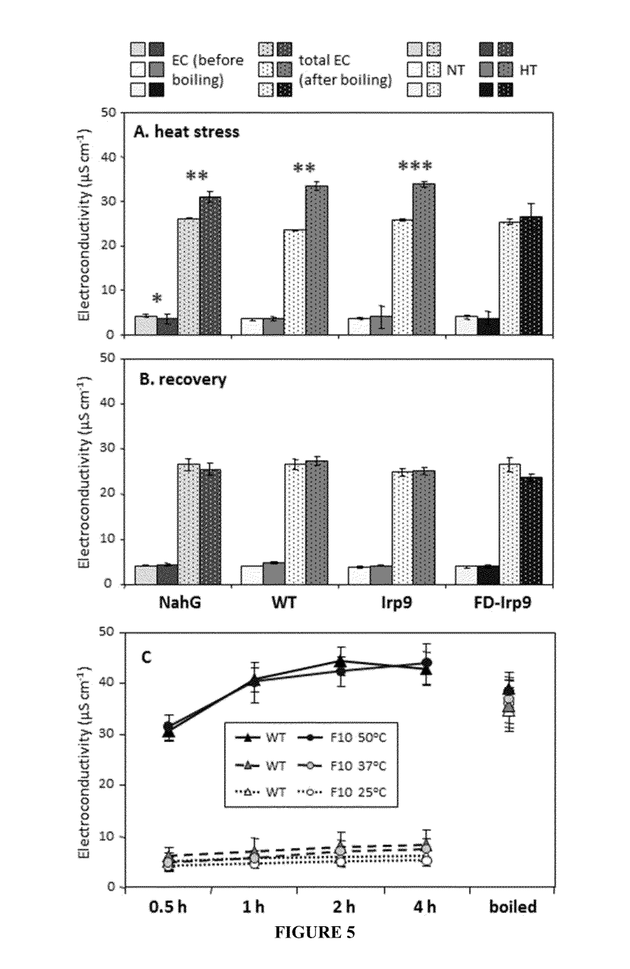

Leaf (LPI-6) tissues from WT and transgenic lines exhibited similar electrolyte leakage regardless of treatment (see FIG. 5A-B), suggesting the absence of any transgenic or HT effect on plasma membrane integrity. The levels of total cellular electrolytes (released after boiling) increased by HT treatments in WT, Irp9 and NahG lines, but not in the FD-Irp9 plants (see FIG. 5A-B), suggesting altered cellular metabolism in response to HT. The lack of genotypic differences in electrolyte leakage was confirmed in a separate test using leaves from greenhouse-grown WT and F10 plants (see FIG. 5C). Together, the data suggested that neither SA nor HT caused cellular membrane damage, but that SA-overproducing plants exhibited lower total cellular electrolytes than the other plant lines during HT growth, indicative of distinct metabolic adjustments.

### Altered Leaf Soluble Phenylpropanoid Composition by SA Perturbation

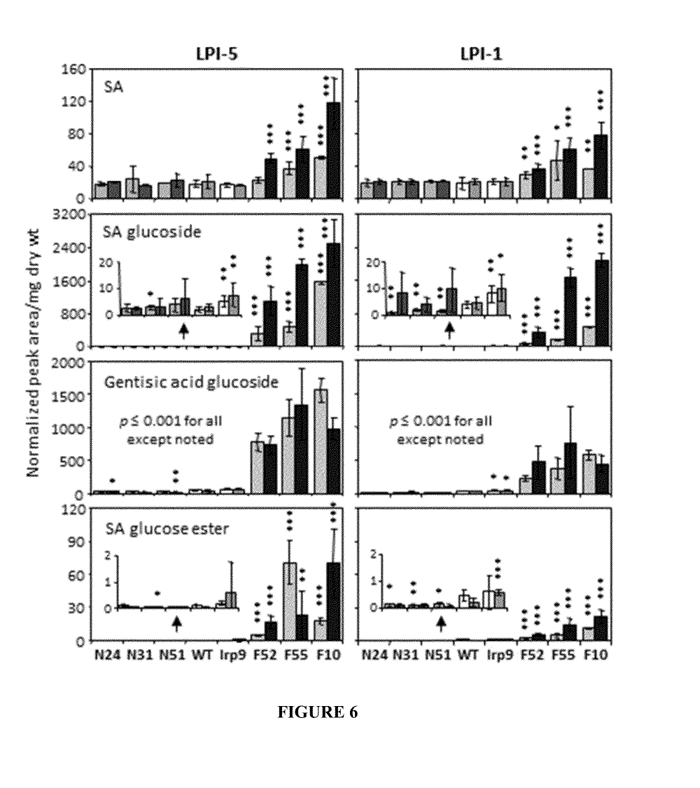

Metabolite profiling of young source leaves (LPI-5) from heat-treated and unstressed plants (WT, I6, I8, F10, F52, F55, N24, N31 and N51) was performed using LC-TOF/MS and GC-MS to gauge the metabolic responses to SA manipulation and/or temperature regime. The genotypic differences (relative to WT) observed from preliminary screening for SA-related metabolites were confirmed. Under NT, the SAG increases were most pronounced in F10 (˜800-fold), followed by F55 and F52 (258- and 165-fold, respectively) (FIG. 6). GAG levels increased by ˜17- to 34-fold, while free SA levels rose slightly (1.3- to 2.7-fold) in these lines. Statistically significant but small (˜2-fold) increases of SAG and GAG in Irp9 plants were found as well, suggesting low levels of cytosolic chorismate-to-SA conversion. The SA degradation product catechol accumulated as catechol glucoside in NahG plants, at ˜4-fold higher levels than in WT. Heat treatment induced a further increase of free SA and SAG, by up to 4-fold, exclusively in the FD-Irp9 lines. An overall similar pattern was observed for LPI-1, a newly emerged sink leaf, although SA conjugates were detected at slightly lower levels than in LPI-5 (FIG. 6). Because SA and SAG are inter-convertible, both capable of inducing defense gene expression and an oxidative burst, and because both were significantly elevated in the FD-Irp9 lines, the FD-Irp9 plants (or transgenic effects) can be referred to as SA-hyperaccumulating (or SA effects).

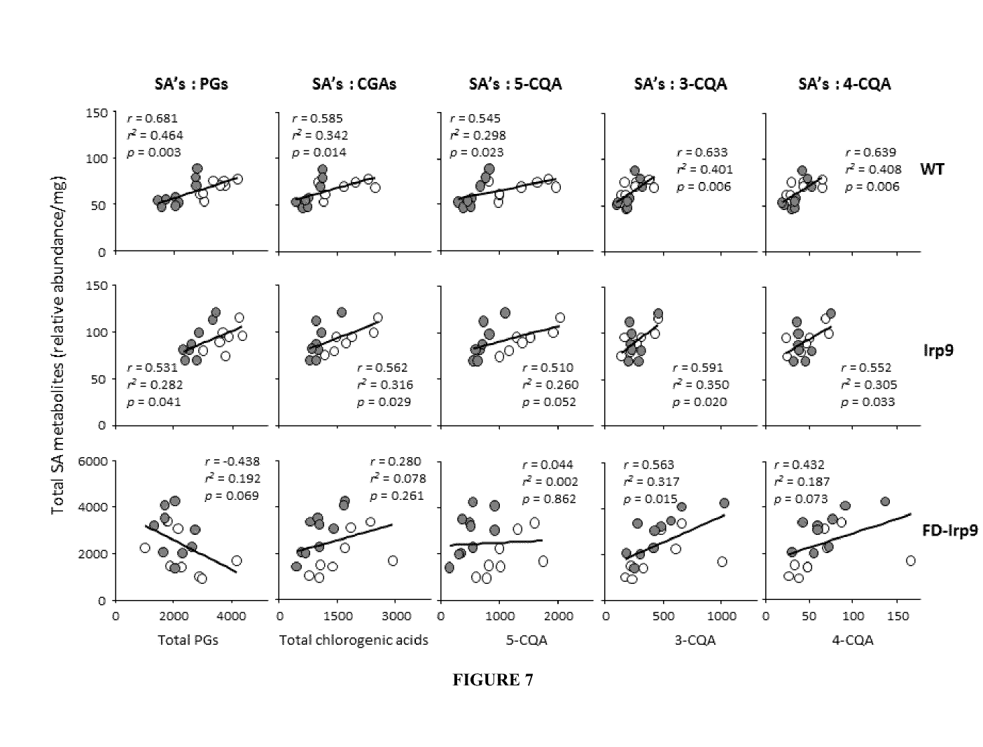

Salicinoids are among the most abundant soluble phenolic metabolites in the experimental Populus clone 717-1B4. At NT, the two major PGs, salicortin and tremulacin, were reduced by ˜20-40% in FD-Irp9, but were increased by ˜20-30% in the NahG lines. HT reduced foliar PG levels overall, but not in FD-Irp9 lines where PG levels were already low prior to HT treatment. Regression analysis showed that total PG levels (salicin, salicortin and tremulacin) correlated positively with total SA (SA and SA-conjugates) in WT and Irp9 lines at both NT and HT, but they correlated negatively in the FD-Irp9 lines (see FIG. 7). The results did not support SA as being a direct precursor of PGs, but were indicative of a metabolic competition between SA and PG accrual. Levels of chlorogenic acids (including caffeoylquinic acid isomers 3-CQA, 4-CQA and 5-CQA), another class of abundant soluble phenolics, were also reduced by heat. Unlike PGs, however, levels of chlorogenic acids correlated positively with SA metabolites in all genotypes, although the trend was weak in FD-Irp9 lines (see FIG. 7). Further analysis of individual isomers revealed two distinct patterns in response to HT or SA perturbation (see FIG. 7). While the predominant 5-CQA was sensitive to HT but insensitive to SA, the reverse was true for the two less abundant isomers 3-CQA and 4-CQA, pointing to a potential role of chlorogenic acid isomerization in response to elevated SA.

### Altered Primary Metabolite Responses to HT in SA-Hyperaccumulating Plants

TCA cycle intermediates citrate, malate, 2-oxoglutarate, succinate and fumarate did not differ between genotypes under NT. HT treatment increased the levels of TCA cycle intermediates, but only in WT, Irp9 and NahG poplars. TCA metabolite levels remained largely unchanged in the FD-Irp9 lines at HT, reminiscent of the pattern observed for total cellular electrolytes (see FIG. 5). Inositol, xylitol, and, to a smaller extent, galactitol were elevated by HT regardless of genotype. The increases of these metabolites therefore represent a general stress response. Sucrose levels did not change significantly among genotypes or due to temperature. The predominant hexoses (glucose and fructose) and pentose (xylose) were reduced in the WT, Irp9 and NahG plants during HT growth, by ˜70%. However, monosaccharide levels were constitutively low in FD-Irp9 plants at NT, and did not decrease further at HT.

### Distinct Correlation Networks Between SA and Phenylpropanoid Metabolites

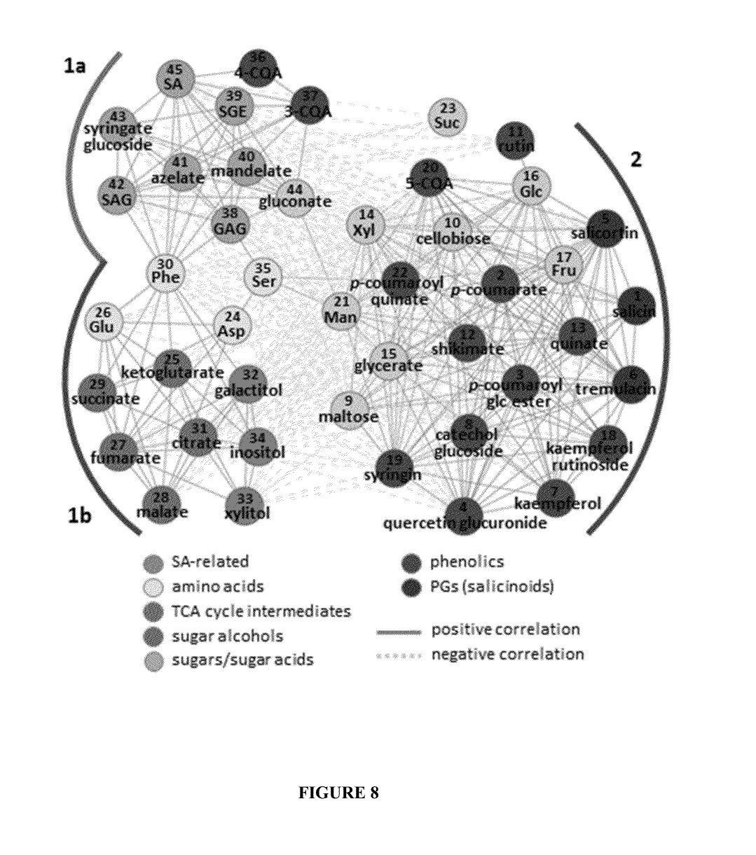

A panel of 45 metabolites that exhibited altered abundance in response to temperature and/or SA manipulation, including those described above, was subjected to correlation analysis across all samples. Two distinct modules were identified, represented by SA-related metabolites and PGs/phenylpropanoids, respectively (FIG. 8). The SA-related metabolites (denoted as group 1a in FIG. 8) were positively correlated, via Phe as a key connector, with amino acids, TCA cycle intermediates and sugar alcohols (group 1b), but negatively with most of the phenylpropanoids and soluble sugars (group 2). PGs, in general, showed positive correlations with other phenylpropanoids and soluble sugars, consistent with a coordinated synthesis utilizing both phenylpropanoid skeletons and hexoses from primary carbon pathways. The differential behaviors between chlorogenic acid isomers mentioned above were also captured in the correlation network. While 5-CQA was positively correlated with other phenylpropanoids in group 2, 3-CQA and 4-CQA were co-regulated with SA metabolites in group 1a (FIG. 8). Four other metabolites in group 1a also exhibited strong correlations with SA metabolites, and they accumulated preferentially in the FD-Irp9 lines. One was identified as putative syringic acid glucoside by LC-TOF/MS (see FIG. 2E). Its level was 10- to 50-fold higher in the FD-Irp9 lines and −80% lower in the NahG lines compared to WT at NT. Two other metabolites were identified by GC-MS exclusively in FD-Irp9 samples. One was related to mandelic acid, while the other was confirmed by authentic standard as the aliphatic signaling molecule azelaic acid (Jung et al., 2009). Gluconic acid was the only other structurally-unrelated metabolite (besides azelaic acid) that correlated strongly with all SA metabolites. It was present in all plants but at significantly higher levels in the FD-Irp9 lines. The nature of these compounds, ranging from phenolic acid and glucoside to fatty acid and hexonic acid, suggested that they have distinct origins, either as SA-derived or SA-stimulated metabolites.

### Overlapping Metabolic Responses to SA and Heat Treatment

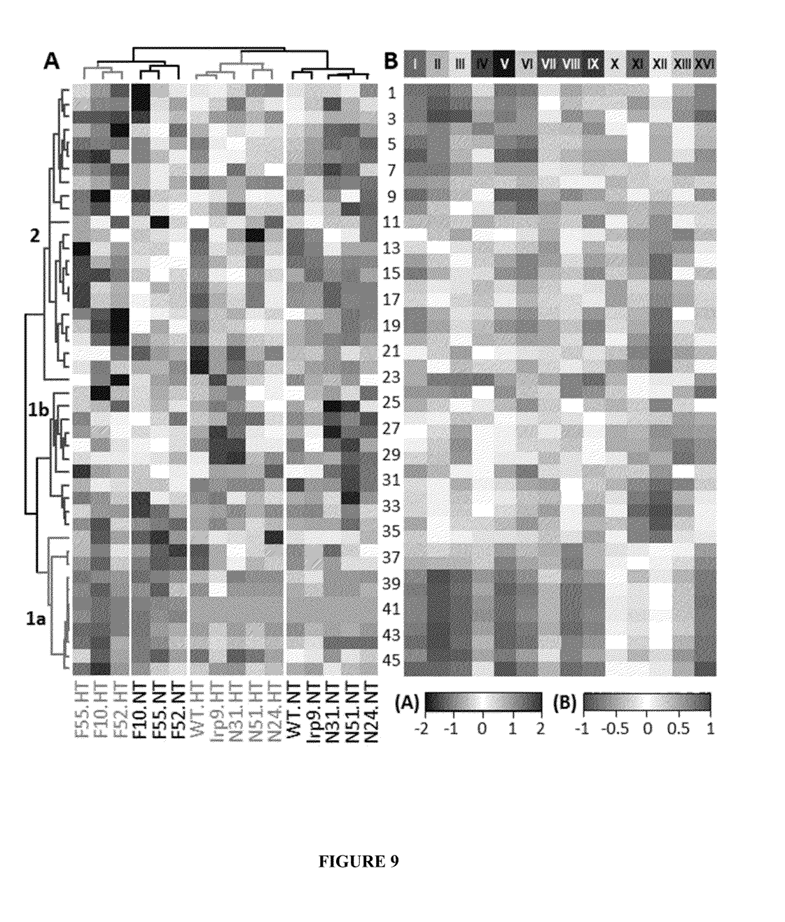

Hierarchical clustering analysis was performed to identify informative patterns of metabolic change among genotypes and/or treatments (FIG. 9). Irp9 and NahG samples clustered with WT into two distinct branches according to temperature treatments (NT vs. HT). The FD-Irp9 samples also formed temperature-dependent groups that clustered separately from the WT, Irp9 and NahG samples. Overall, HT triggered extensive metabolic changes in the WT, Irp9 and NahG plants, but the temperature-induced changes in FD-Irp9 lines were less striking. In fact, many metabolites in the FD-Irp9 plants under NT exhibited patterns that resembled those of HT-treated WT, Irp9 or NahG plants, and included soluble sugars, PGs, 5-CQA and other phenylpropanoids (corresponding to group 2 in FIGS. 8 and 9). Most of these metabolites showed decreased abundance in response to HT and/or high SA, consistent with their negative correlation with SA metabolites (FIG. 8). Group 1b encompasses amino acids as well as the heat-responsive metabolites (TCA intermediates and sugar alcohols) mentioned above. Group 1a includes primarily SA-related metabolites with biased accumulation in the FD-Irp9 lines. Together, the metabolite results showed that SA-hyperaccumulation led to a host of metabolic adjustments, involving primary and secondary metabolites, as well as antioxidants and potential signaling molecules.

### SA-Mediated Transcriptome Responses Also Recapitulated Heat-Induced Responses

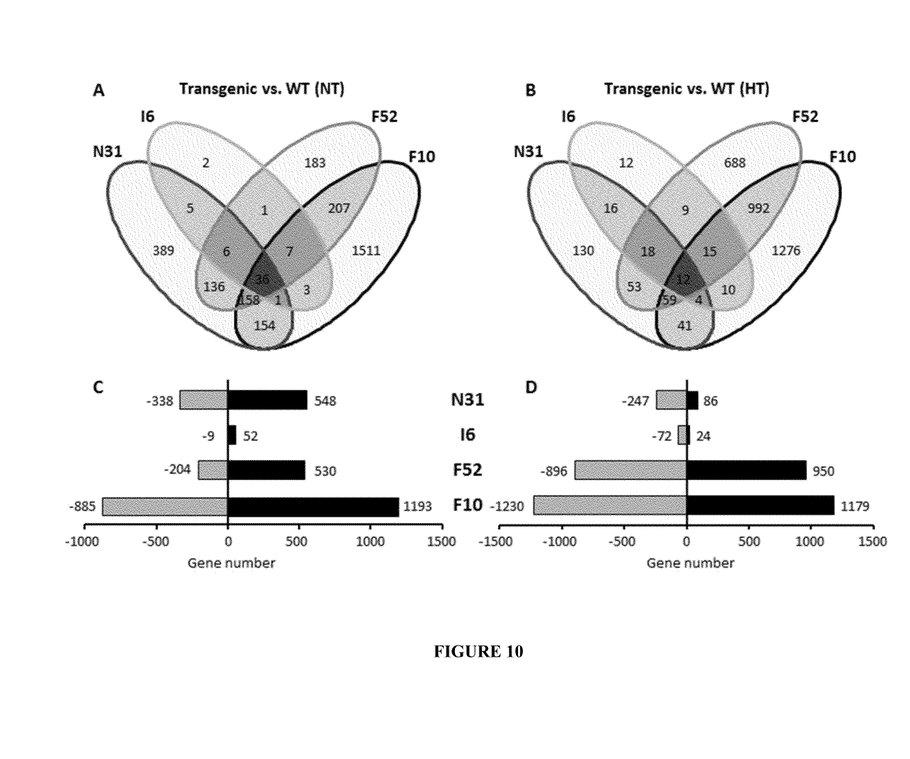

Microarray experiments were performed to gauge the transcriptional responses of young source leaves (LPI-5) to SA manipulation, using WT and one Irp9 (16), one NahG (N31), and two FD-Irp9 (F10 and F52) transgenic lines with contrasting SA levels. Genes that were differentially expressed (DE) due to SA perturbation (transgenic vs. WT) or temperature treatment (NT vs. HT) were identified using adjusted p≦0.05 and fold-change≧2 (see Methods). Compared with WT, I6 exhibited the fewest DE genes under either NT or HT (see FIG. 10), consistent with its overall metabolite profile similarity to WT. The numbers of DE genes were greater for N31 and F52, but the DE response was most conspicuous for the F10 line. The difference between F10 and F52 was in accordance with their SA levels, and thus reflected a dose-dependent transcriptional response. At HT, the number of DE genes decreased in N31, but the number increased considerably in F52, approaching that of F10 (see FIG. 10). In general, more genes were up-regulated than down-regulated in all transgenics at NT, but DE patterns were more complex under HT.

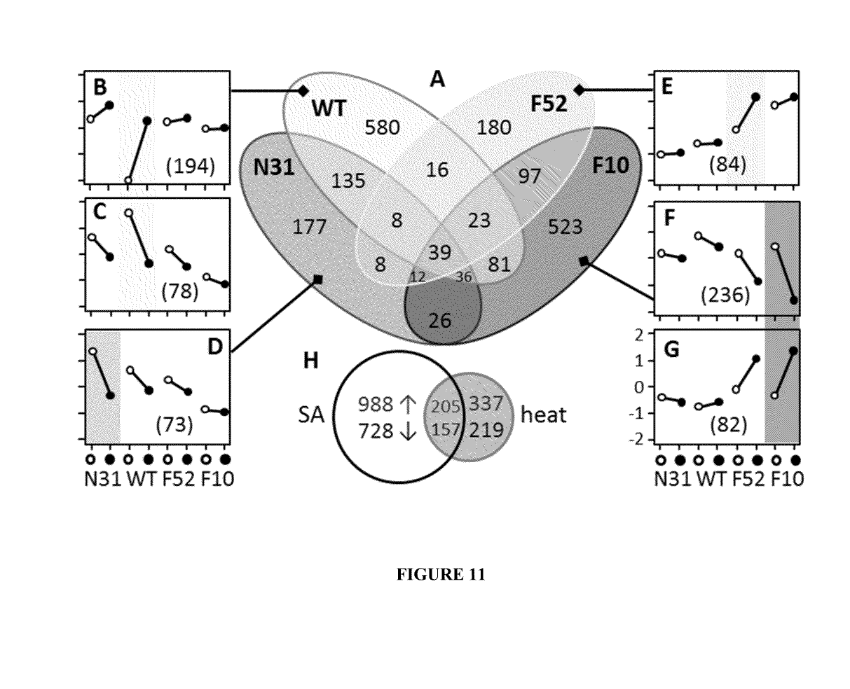

Gene expression responses to HT in the different genotypes were summarized in a Venn diagram (FIG. 11A). The 16 line was excluded, because it was least affected metabolically and transcriptionally. Only a small number of genes showed significant differences in all genotypes, with 32 up-regulated and seven down-regulated (FIG. 11A). Genes encoding heat-shock proteins and vegetative storage proteins predominated in the up-regulated group. Between 177 and 580 genes showed genotype-specific DE response to heat, and these genes were subjected to clustering analysis to identify representative expression patterns within each group (FIG. 11B-G). Across genotypes, the vast majority of these genes responded similarly to HT (i.e., up- or down-regulation), varying only in degree. For example, the largest cluster F included 236 genes that showed HT-reduced expression in all genotypes, but only the differential in the F10 line satisfied our DE criteria (FIG. 11F). The transcript levels of many of these genes in unstressed FD-Irp9 plants resembled those in heat-treated WT. Accordingly, considerable overlap between was found between DE genes that were sensitive to SA (F10 vs. WT) and to heat (NT vs. HT in WT): about 40% of the heat-responsive DE genes in WT were similarly up- or down-regulated by SA-hyperaccumulation (FIG. 11H). The results were consistent with findings from metabolite profiling, and suggested that SA-hyperaccumulation recapitulated the HT-induced transcriptional responses as well.

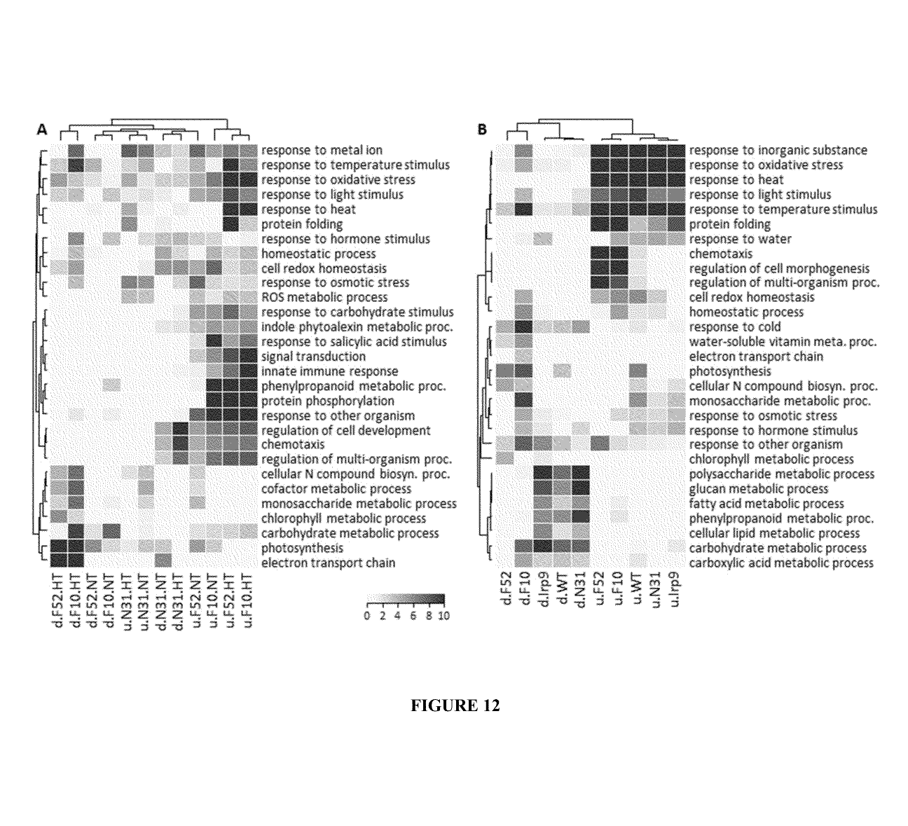

Gene Ontology (GO) enrichment analysis was performed to identify cellular functions that were over-represented among SA- and/or heat-responsive genes. GO categories associated with biotic and abiotic stress responses and signal transduction were significantly over-represented among genes that were up-regulated in the FD-Irp9 lines, while genes related to photosynthesis and carbohydrate metabolism were over-represented among those that were down-regulated in these lines (see FIG. 12A). Analysis of heat-responsive genes revealed two distinct clusters according to their expression patterns (up vs. down-regulation), with high-SA (F10 and F52) and low-SA (WT, I6 and N31) lines forming separate subgroups within each cluster (see FIG. 12B). GO categories associated with oxidative stress responses, redox homeostasis and protein folding were over-represented among heat-stimulated genes. Genes associated with polysaccharide and phenylpropanoid metabolism were repressed by heat, especially in low-SA lines (see FIG. 12B), consistent with findings from the metabolite analysis.

### Weighted Correlation Network Analysis Identified SA-Regulated Gene Modules

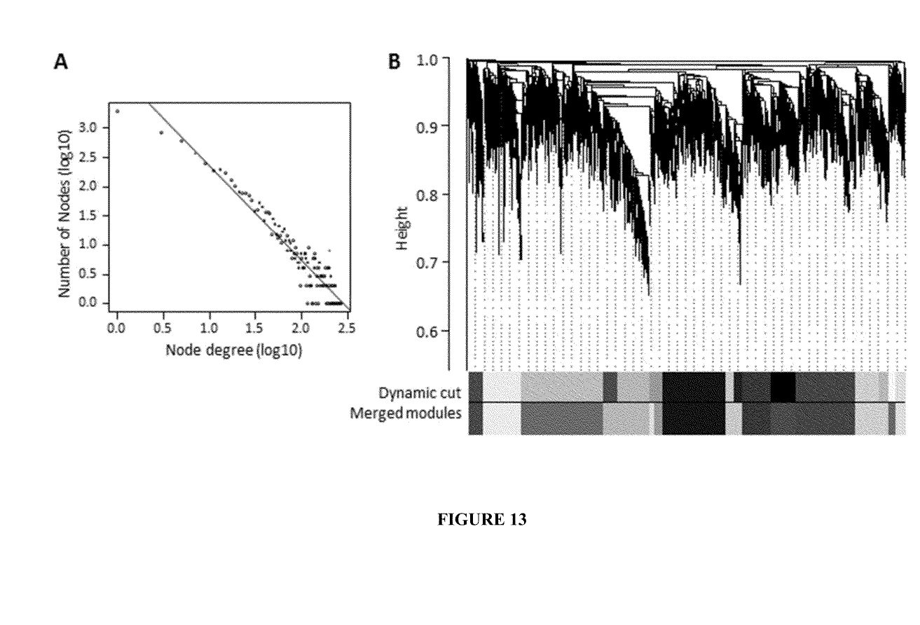

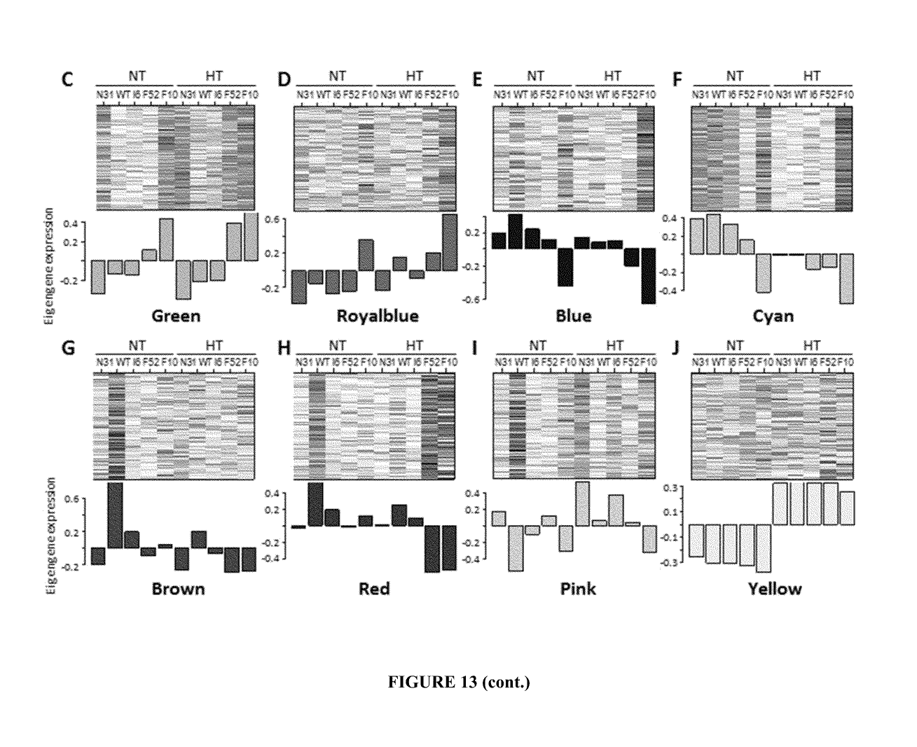

Transcriptional interactions of SA- and/or heat-sensitive genes were investigated using the eigengene weighted correlation network approach (Langfelder and Horvath, 2008). A less stringent procedure was used to obtain 8,570 DE genes, which included the two bacterial transgenes, Irp9 and NahG. The marker metabolite SAG was also added as a non-gene node (using normalized relative abundance of corresponding samples) in order to facilitate identification of genes correlating with SA changes. The degree distribution of the resultant network follows the power law (see FIG. 13A), a common property of scale-free networks (Barabási and Albert, Science 286: 509-512 (1999)). Fourteen co-regulation modules were identified (see FIG. 13B), and the expression profile of each module is represented by the module eigengene (first principal component). Correlation analysis of module eigengenes with metabolite traits across genotypes and treatments allowed identification of co-varying gene-metabolite responses (FIG. 9). For instance, SA-related metabolites (group Ia) showed strong positive correlations with the (I), (II), and (III) modules of the network, but were negatively correlated with the (V) and (VI) modules (FIG. 9). Specifically, genes in the (II) module exhibited an SA-dependent expression profile, lowest in N31 and highest in F10 (see FIG. 13C), and not surprisingly, both the SAG and Irp9 nodes were captured in this module. PGs (metabolite no. 1, 5, 6 in FIG. 9) were found to correlate positively with the (V) and (VI) modules but negatively with the (I) and (II) modules, in accordance with the inverse relationship between PGs and SA metabolites described above. The (XII) module exhibited a temperature-dependent regulation regardless of genotype (see FIG. 13J). GO enrichment analysis provided further support for differential functional associations of the various modules. For example, genes associated with defense response and signal transduction were greatly enriched in the SAG-containing (II) module. Genes associated with heat response and protein folding were enriched in the (XII) module, consistent with their transcriptional induction by heat.

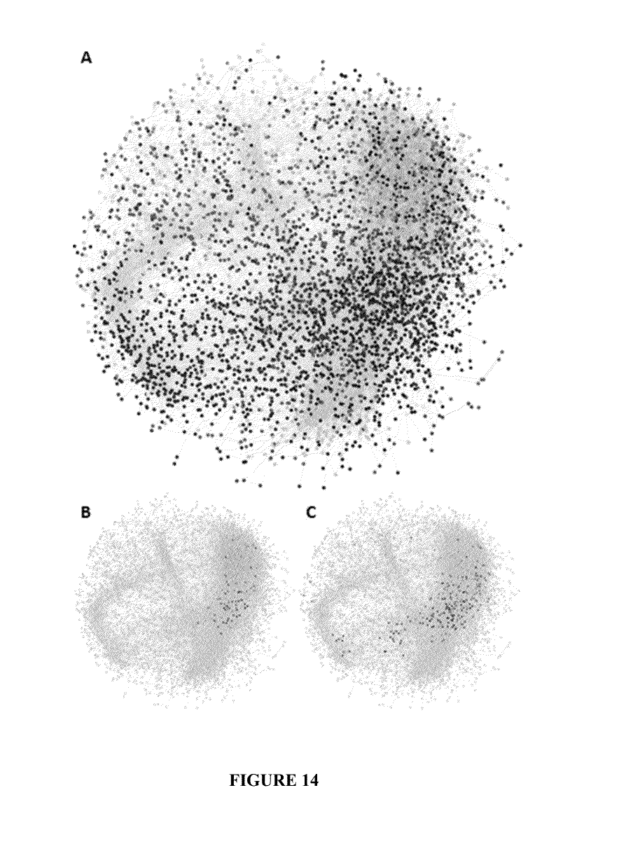

Research in biological networks has shown that highly connected nodes (“hubs”) are usually enriched with genes that play important roles in controlling the behavior of the system under investigation (Jeong et al., Nature 411: 41-42 (2001)); Carter et al., Bioinformatics 20: 2242-2250 (2004)). To identify key players in this reconstructed network, genes were ranked based on their total connectivity (k) in the network, and the top 5% most densely connected nodes were designated as hubs (Basso et al., Nature Genetics 27: 382-390 (2005)). The k of the hub genes ranged from 162.3 to 314.7, several-fold greater than the average (31.1) or median (49.1) k of all nodes in the weighted network. Collectively, these hubs participated in nearly half of the total connections (45,501 of 97,162 edges) in the network, with an average of 103 connections per hub gene. When the gene network was visualized in Cytoscape (FIG. 14A), these hubs were found in a localized cluster (FIG. 14B), in sharp contrast to the more scattered distribution of the top 5% nodes from each module (FIG. 14C). More than 75% of the hubs, including SAG, were from the green (II) module (Table 3), suggesting that SA-responsive genes were drivers of the network.

TABLE 3
Module assignment of hub genes from the total
as well as FD-Irp9 and Wt subnetworks.

| | | | FD-Irp9 | WT | | |
|---|---|---|---|---|---|---|
| | | Total | sub- | sub- | DC (k_{FD-Irp9} − k_{WT}) | |
| Module^{a} | node # | network | network | network | k gain | k loss |
| Royalblue (I) | 1640 | 25 | 127 | 25 | 136 | 24 |
| Green (II) | 932 | 330 | 55 | 4 | 60 | 7 |
| Blue (V) | 1235 | 38 | 118 | 7 | 144 | 14 |
| Cyan (VI) | 346 | 14 | 21 | 22 | 10 | 9 |
| Brown (VII) | 1179 | 4 | 11 | 160 | 5 | 201 |
| Red (IX) | 493 | 14 | 13 | 9 | 11 | 5 |
| Yellow (XII) | 746 | 0 | 21 | 54 | 6 | 41 |
| Pink (XIII) | 665 | 0 | 19 | 144 | 2 | 119 |
| Total^{b} | 8751 | 438 | 438 | 438 | 438 | 438 |

^{a}Only eight modules are shown.
^{b}The numbers are summed from all modules.

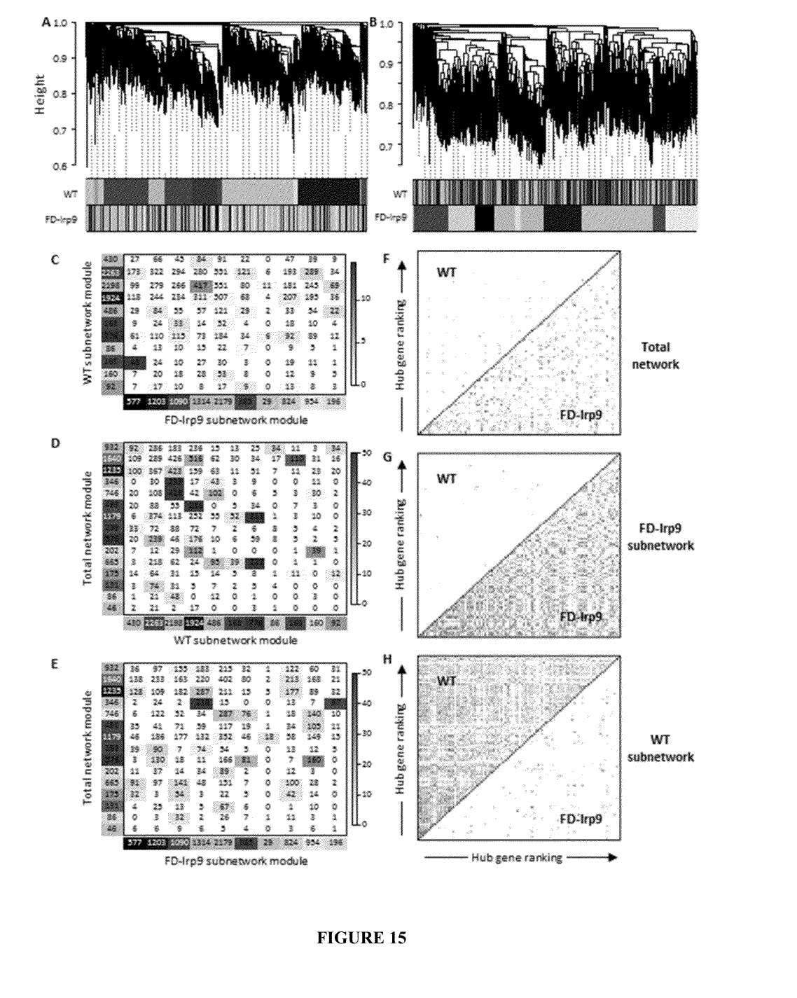

To confirm this observation, and to further explore the effects of SA-hyperaccumulation on gene network rewiring, the data was divided into WT-like (WT, I6 and N31) and high-SA (F10 and F52) groups and two subnetworks were constructed (hereafter, referred to as WT and FD-Irp9 subnetworks, respectively) for comparative analysis. The two subnetworks appeared quite distinct, with little conservation across modules (see FIG. 15A-C). When compared against the total network, an overall greater degree of module conservation was observed with the WT than with the FD-Irp9 subnetwork (see FIG. 15D-E). However, the hub genes from the total network were more similar to those from the FD-Irp9 subnetwork than from the WT subnetwork (see FIG. 15F-H), suggesting that the relatively small number of hub genes contributed significantly to SA-modulated network rewiring.

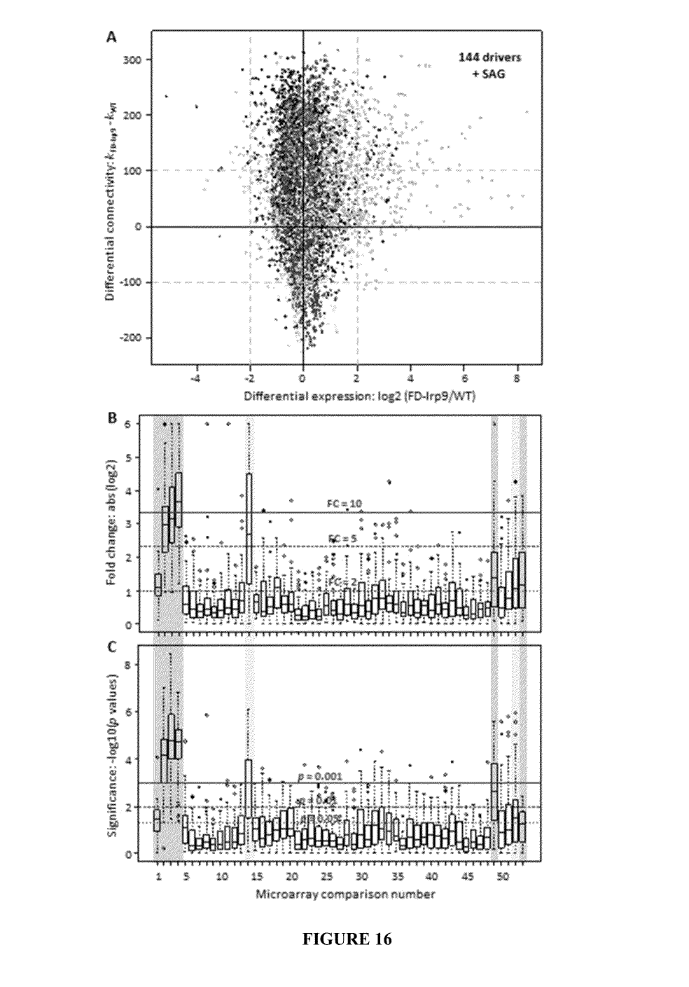

Analysis of differential connectivity (DC) between the two subnetworks (determined as k_{FD-Irp9}−k_{WT}) showed that the top-5% nodes that gained connectivity (i.e., with more co-regulating genes) in the FD-Irp9 subnetwork overlapped substantially (˜70%) with the hub genes there. These genes corresponded primarily to the SA-correlated royal-blue (I), green (II) and blue (V) modules in the total network (Table 3). Conversely, a majority of hub genes (˜55%) in the WT network lost their connectivity in the FD-Irp9 network (Table 3). Using an arbitrary DC cutoff of 100 and a DE (fold-change) cutoff of ≧4 between the two plant groups, a total of 144 genes (plus the SAG node) showed increased expression as well as increased connectivity in the FD-Irp9 plants, 80% of which were from the (II) module (FIG. 16A, top right corner). These genes thus represent potential drivers contributing to the widespread transcriptomic changes, involving both differential expression as well as altered network connectivity, due to SA hyper-accumulation.

### Potential Drivers of the SA-Modulated Network

The group of 144 potential drivers was enriched with receptor-like protein kinases (RLKs) and various transporters, oxidoreductases and transcription factors, most of which (˜73%) were also hubs in the total network.

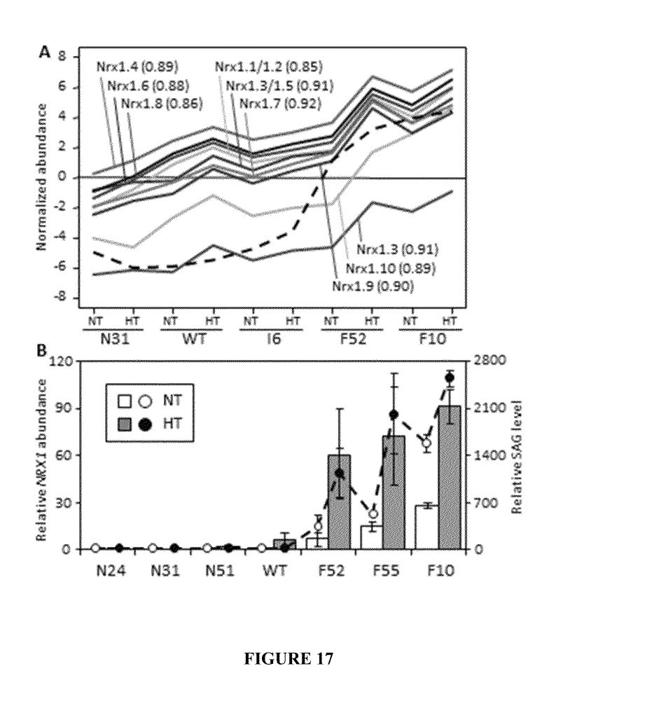

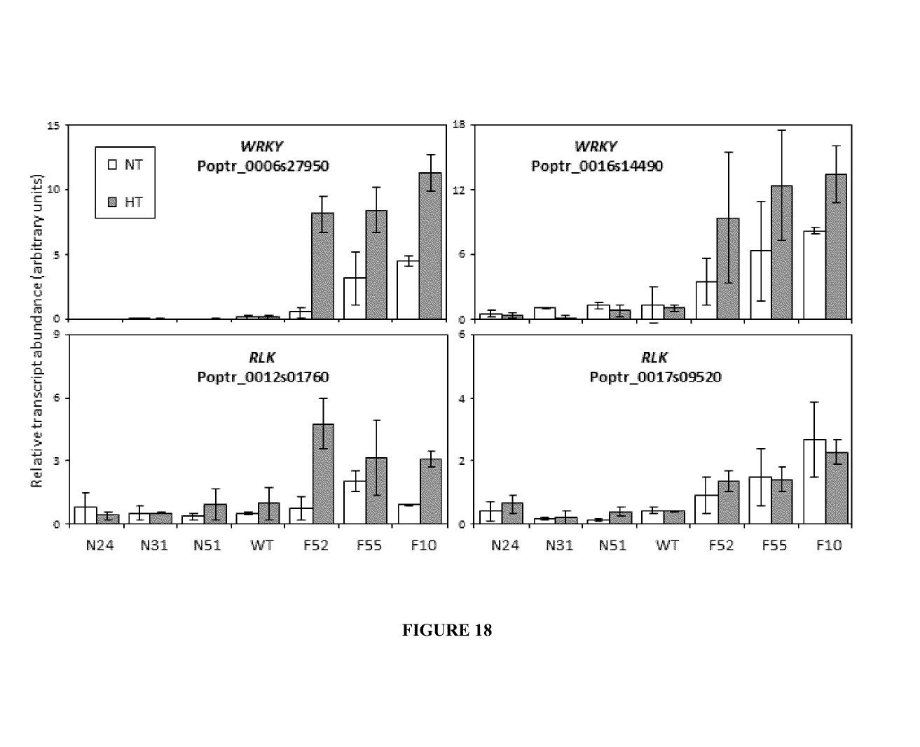

RLKs belong to the protein kinase superfamily with important functions in defense signaling Of the 308 RLKs captured in this network (i.e., with differential expression among genotypes or treatments), a majority of them (˜77%) were associated with SA-correlated modules, especially the (II) module where RLKs accounted for ˜13% (132) of its nodes. The large number of RLKs with increased expression as well as increased network connectivity in high-SA poplars suggested their roles in the reprogramming of signaling pathways in these plants. The group of oxidoreductases included the entire Populus NRX1 subfamily—the only family of small redox proteins (glutaredoxins [GRXs] or thioredoxins [TRXs]) represented among the drivers. All nine NRX1 probes on the microarray exhibited an SA-dependent expression pattern that was also confirmed by qRT-PCR (FIG. 17). Their involvement in SA-network rewiring represents a previously undescribed role of plant NRX1 in stress-associated redox regulation. Among the 144 drivers were orthologs of Arabidopsis genes that are known to be involved in the SA defense signaling pathway. Examples include orthologs (POPTR_{—}0014 s03260 and POPTR_{—}0019 s03090) of patatin-like phospholipase A₂ (Scherer et al., 2010) and several defense-associated WRKYs that exhibited SA-stimulated expression in this study. The observed transcriptional responses of representative RLK (Poptr_{—}0012 s01760 and Poptr_{—}0017 s09520) and WRKY (Poptr_{—}0006 s27950 and Poptr_{—}0016 s14490) genes were confirmed by qRT-PCR (see FIG. 18). Overall, the network analysis provided a glimpse of previously reported as well as new SA-sensitive components of redox regulation and inducible defense signaling pathways.

Because SA-hyperaccumulating poplar exhibited constitutive metabolic and transcriptional responses that resembled those of heat-treated poplars, it was reasoned that the group of potential drivers may modulate the general oxidative stress responses of Populus. To find support for this hypothesis, published Populus leaf microarray data sets derived from oxidative stress experiments were studied. The analysis encompassed 48 paired comparisons (stressed vs. unstressed samples) of drought (Wilkins et al., Plant Journal 60: 703-715 (2009)); Cohen et al., BMC Genomics 11: 630 (2010); Hamanishi et al., Plant, Cell and Environment 33: 1742-1755 (2010)); Raj et al., PNAS 108: 12521-12526 (2011)); Chen et al., Plant Mol. Biol. Rep. doi: 10.007/s11105-11013-10563-11106 (2013)), wounding (Yuan et al., PNAS 106: 22020-22025 (2009)), pathogen (Azaiez et al., Journal of Chem. Ecology 36: 286-297 (2009)) and ozone responses (Street et al., Environ. Pollut. 159: 45-54 (2011)). After quality control filtering, DE of the 144 driver genes, when present, was assessed by fold-change and p values and shown in box-and-whisker plots for all sample pairs (FIG. 16B-C). Data from the present study were included as reference. As expected, expression of these driver genes showed significant and large fold-change differences due to elevated SA (nos. 1-4, FIG. 16B-C). The responses followed an SA dose-dependent trend: stronger in F10 (nos. 3-4) than F52 (nos. 1-2), and at HT (nos. 2 and 4) than NT (nos. 1 and 3). These genes also responded strongly to wounding (no. 14), drought (no. 49), pathogen (no. 52) and ozone (no. 53, FIG. 16B-C), sometimes in a dose-dependent manner.

Specifically, of the large number of drought samples analyzed, a significant response of the driver genes was observed only in P. simonii (no. 49), a hardy species indigenous to northern China (Wang et al., American Journal of Botany 99: e357-e361 (2012)). Similar results were obtained for the 438 network hub genes, but the overall responses were weaker than those of the driver genes (see FIG. 19), consistent with greater responsiveness of the driver genes. A common theme among the biotic (pathogen) and abiotic (drought, wounding and ozone) stressors is their ability to trigger oxidative responses in plants (Kovtun et al., PNAS 97: 2940-2945 (2000)). These results suggest that elevated oxidative state, due either to genetic variation, SA-overproduction or stress manipulation, primes plants to elicit potent oxidative responses as evidenced by elevated expression of the driver genes. Thus, this analysis provided independent support for the involvement of the driver genes in poplar oxidative stress responses, including those induced by elevated SA.

Metabolic and Transcriptome Reprogramming in High-SA Poplars Resembles that Induced by Oxidative Stress

These results provide multiple lines of evidence to support a direct role of SA in eliciting sustained oxidative responses. Constitutively elevated SA promoted transcriptional and metabolic changes in transgenic FD-Irp9 lines that resembled those observed in WT and WT-like (Irp9 and NahG) plants after prolonged growth at HT, a condition known to increase oxidative stress. In particular, there was substantial overlap between HT- and SA-up-regulated genes, with both groups showing similar GO enrichment in oxidative stress responses. While stress metabolite sugar alcohols were increased by HT regardless of genotype, the abundance of glucose, fructose and many of the phenylpropanoids were constitutively low in leaves of FD-Irp9 plants, similar to levels that were observed in heat-treated WT or WT-like poplars. Also, of interest, was the strong correlation of azelaic acid and gluconic acid with SA and SA-conjugates.

Changes in Chlorogenic Acid Metabolism could Provide Protection to Chloroplasts During SA-Induced Stomatal Closure

It was observed that the increases in net photosynthesis and transpiration due to HT were attenuated in lines with constitutively elevated SA (FIG. 3). In addition, stomatal conductance was clearly reduced in FD-Irp9 lines at HT, when SA and SAG levels were further increased (FIG. 6). This, along with elevated levels of SA metabolites (including azelaic acid and gluconic acid), possibly intensified the oxidative state of the FD-Irp9 plants at HT. During HT growth, therefore, reduced photosynthetic capacity, increased glycosylation of SA and elevated oxidative state in the growth-sustained FD-Irp9 plants could have exacerbated a metabolic trade-off at the expense of soluble phenylpropanoids/sugars. Interestingly, chlorogenic acid isomers 3-CQA and 4-CQA were the only phenylpropanoids from the panel that showed positive correlations with SA metabolites, while 5-CQA co-regulated with other phenylpropanoids (FIG. 8). It is possible that the changes in chlorogenic acid pool composition reflect a change in cellular redox due to elevated SA and/or SA-induced stomatal closure. The fact that chlorogenic acids are abundant in Populus leaves suggests a readily available and SA-sensitive pool for local protective responses, perhaps at the subcellular level, in the event of SA-mediated changes in ROS.

### Redox Control Pathways Differ Between Populus and Arabidopsis

The correlation network analysis enabled identification of potential drivers in SA-modulated rewiring of the transcriptional network. Meta-analysis of Populus leaf stress transcriptome showed that these driver genes were also highly responsive to a host of biotic and abiotic oxidative stressors. The most prominent group was RLKs that have been implicated in a wide range of signaling cascades during plant growth, development, and biotic and abiotic responses. Oxidoreductases were also enriched among the drivers in accordance with their importance to redox regulation in stress response and SA signaling NRX1s were the only TRX/GRX genes that exhibited SA-dependent expression and network rewiring in this study. The data provided herein suggest that while some aspects of the SA signaling pathway appear to be conserved between Populus and Arabidopsis, other components have diverged during evolution. In particular, while 2- to 200-fold total SA increases in Arabidopsis negatively affect growth (see Rivas-San Vicente and Plasencia, 2011), multi-fold differences in total SA level did not negatively affect Populus growth. As reported here, total SA increases of up to 1000-fold in transgenic Populus had no apparent effects on plant growth.

In summary, endogenously elevated SA in Populus elicited potent and sustained oxidative responses. Stomatal behavior was altered as was transcriptional and metabolic reprogramming. The high photosynthetic capacity of Populus, and the metabolic flexibility afforded by the dynamic chlorogenic acids pool are both likely to contribute to SA tolerance.

### Example 2

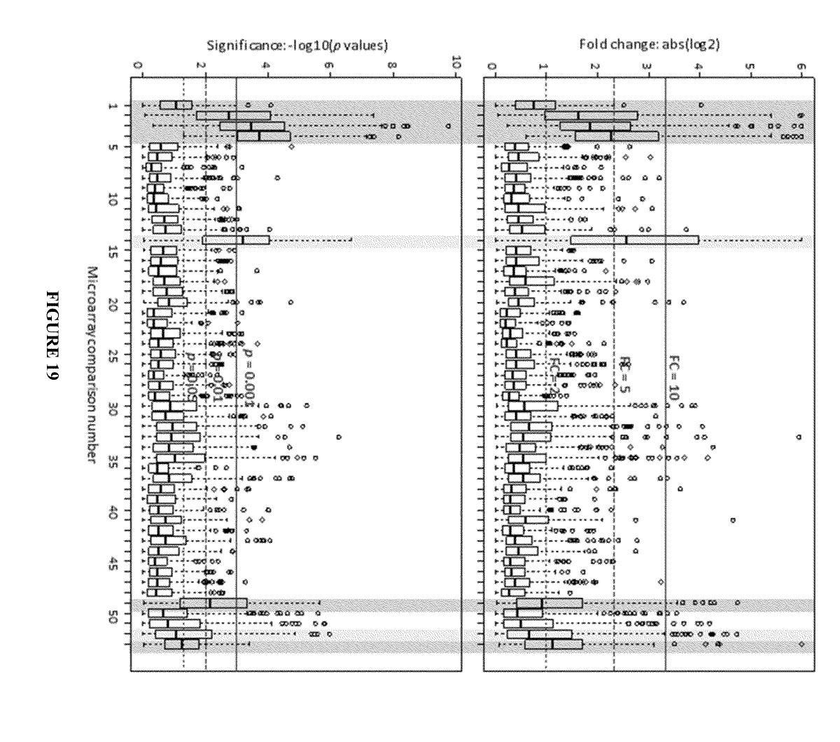

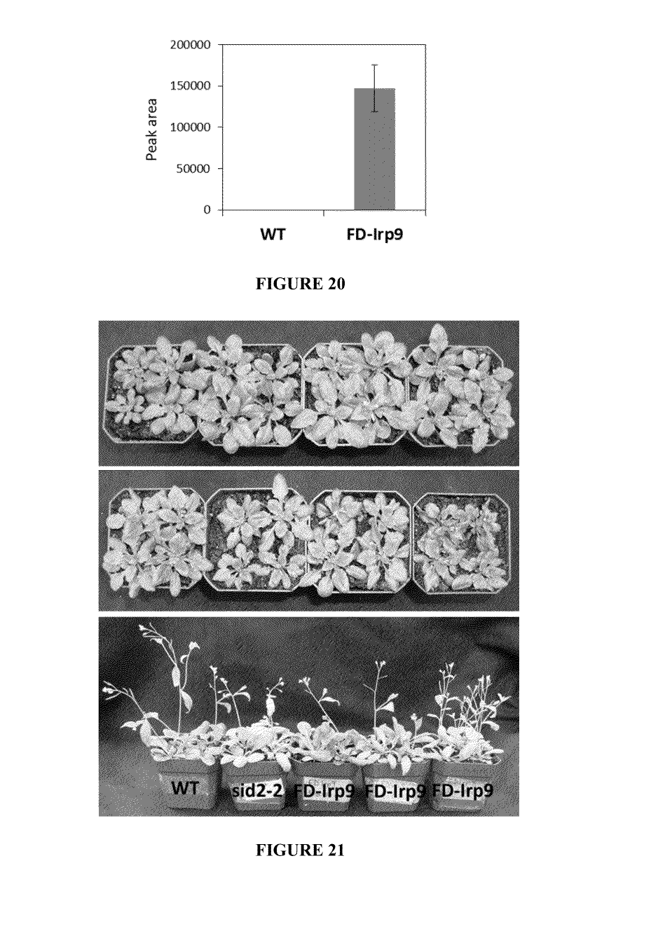

Using the methods described above, transgenic Arabidopsis expressing the plastidic FD-Irp9 gene were made. Transgenic Arabidopsis expressing the plastidic FD-Irp9 gene accumulated elevated levels of SA-glucoside (SAG) with normal growth. Although SAG level is typically undetectable in WT Arabidopsis, as shown in FIG. 20, it is present at very high levels in a representative transgenic line (F-2421). Further, as shown in FIG. 21, plant growth was not affected in transgenic FD-Irp9 Arabidopsis. Top and middle panels show young WT and FD-Irp9 transgenic Arabidopsis seedlings, respectively. The bottom panel shows flowering WT, sid2-2 mutant and FD-Irp9 transgenic Arabidopsis plants. The sid2-2 mutant has a defect in SA biosynthesis. Thus, increased SA levels can be achieved in Arabidopsis plants without compromising growth.

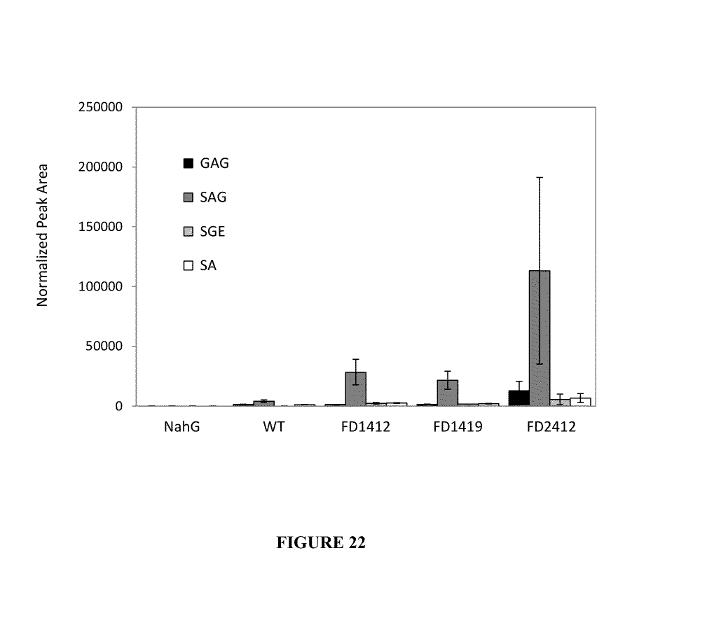

Transgenic Arabidopsis hyperaccumulating SA was obtained from two independent transformation experiments with the FD-Irp9 construct. Three transgenic lines with the highest levels of SA metabolites are shown in FIG. 22. SA=salicylic acid, SAG=SA glucoside, SGE=SA glucose ester, GAG=gentisic acid glucoside.

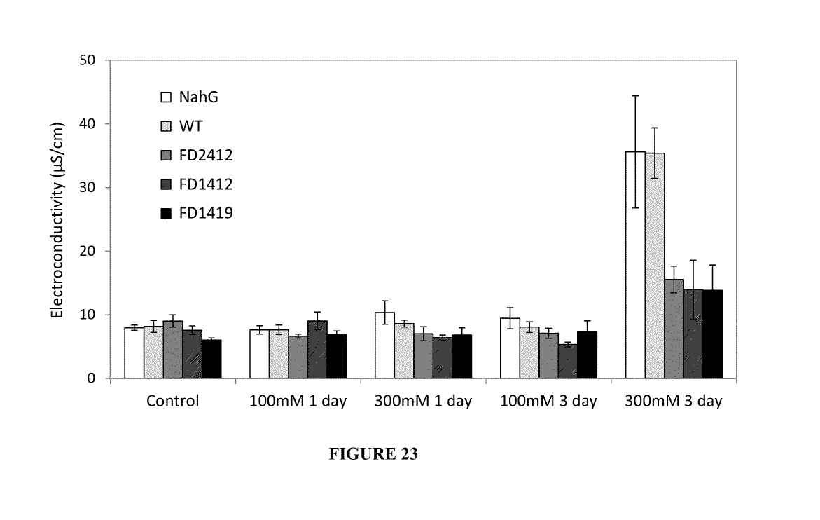

Plants were subjected to salt stress treatments at 100 mM or 300 mM for 1-3 days. Under severe salt stress (300 mM for 3 days), electrolyte leakage of WT and NahG leaves increased by more than 4-fold relative to unstressed leaves. The increase in high-SA plants was ˜2-fold, resulting in significantly lower levels of electrolyte leakage in high-SA plants than in WT and NahG plants (FIG. 23). These results show that hyperaccumulation of SA can confer plasma membrane protection under severe salt stress.

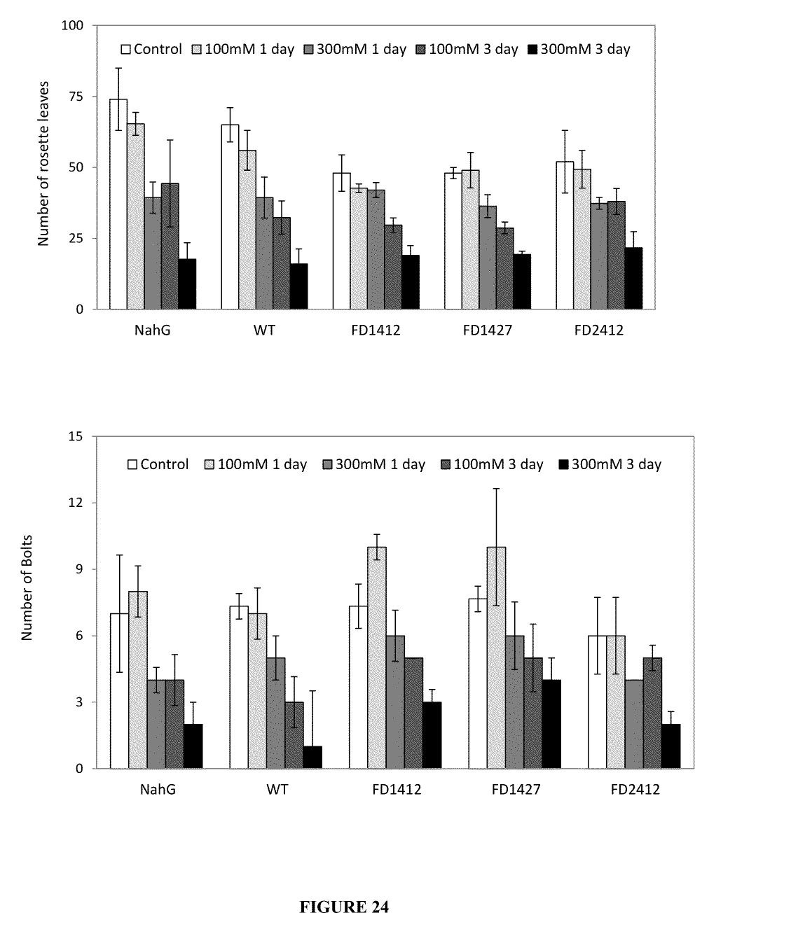

SA-hyperaccumulating plants had fewer leaves than the WT and NahG plants, but similar or slightly higher number of bolts (flowering stems) under normal growth conditions (FIG. 24, top and bottom panels). This shows that SA hyperaccumulation can affect biomass allocation.

Salt stress had negative effects on growth (number of leaves and bolts) in all plants, but the effects were stronger on WT and NahG plants than on high-SA lines. As a result, leaf number became similar between genotypes under prolonged (3-day) or high level (300 mM) of salinity (FIG. 24, top and bottom panels). Some WT and NahG plants failed to produce flowering stems, and had fewer siliques, while silique production of the high-SA plants were not affected by salinity. Thus, SA over-producing plants showed improved salt tolerance and reproductive fitness.

### Example 3

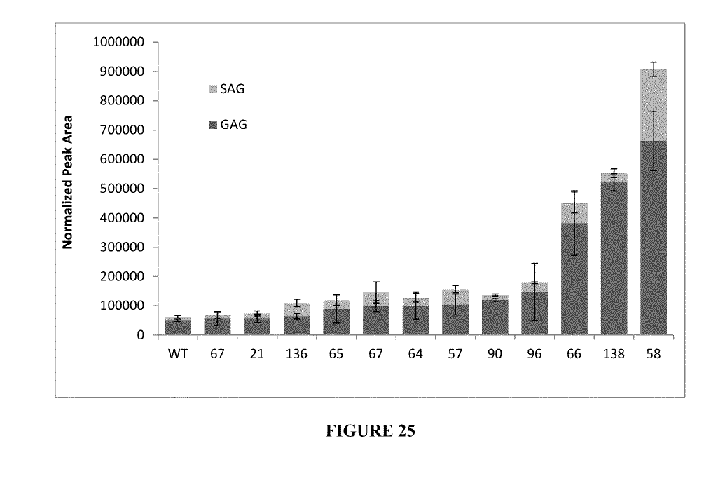

Transgenic soybean with increased SA levels were also produced by expressing the FD-Irp9 construct. FIG. 25 shows representative high-performing lines, with up to 15-fold higher levels of SA glucoside (SAG) and gentisic acid glucoside (GAG). Primary transformants did not show obvious morphological phenotypes. These data collectively demonstrate the wide applicability of the improved method for increasing SA levels in both woody and herbaceous species, including agronomically important crops.

Sequences

(SEQ ID NO: 1)AFN32483|Klebsiella oxytoca E718
MKISEFLHLALPEEQWLPMISGVLRQFGDEECYVYERQPCWYIGRGCQAQ
LQINADGTQATFIDDAGEQKWAVDSITDCARRFMTHPRVRGCRVYGQVGF
NFAAHARGMAFDAGEWPLLTLTAPREELIFEKGNVTVYADSADGCRRLCE
WVKEVDTATPCGPMVVDTALDGEAYKQQVARAVSAIRRGDYVKVIVSRAI
PLPARIDMPATLLYGRQANTPTRTFMFRQQGREALGFSPELVMSVTGKKV
VTEPLAGTRDRMGDMAHNQANERELLHDGKEVLEHILSVKEAIAELEAVC
QPGSVVVEDLMSVRQRGSVQHLGSGVSGQLAEDKDAWDAFTVLFPSITAS
GIPKKAALNAIMQIEKTPRELYSGAILLLEDTRFDAALVLRSVFQDSQRC
WIQAGAGIIEQSTPERELTETREKLASIAPYLKVPA
(SEQ ID NO: 2)AGJ85202|Raoultella ornithinolytica
B6
MKISEFLHLALPEEQWLPTISGVLRQFGDEECYVYERQPCWYLGKGCLAR
LHINADGTQATFIDDAGEQKWAVDSIVDCARRFMAHPQVQGRRVYGQVGF
NFAAHARGIAFDAGEWPLLTLTVPREELVFEKGNVTVYTDSAEGCRRLCE
WVKEASTTTRGDSMAVDTALNGEVYKQQVARAVAEISRGEYVKVIISRAI
PLPSRIDMPATLLYGRQANTPTRSFMFRQQGREALGFSPELVMSVTGNKV
VTEPLAGTRDRMGSPEQNKAKETELLHDSKEVLEHILSVKEALAELAVVC
RPGSVVVEDLMSVRKRGSVQHLGSGVSGQLAENKDAWDAFTVLFPSITAS
GIPKNAALNAIMRIEKTPRELYSGAILLLEDARFDAALVLRSVFQDSQRC
WIQAGAGIIAQSTPERELTETREKLASIAPYLMVSE
(SEQ ID NO: 3) CAX65486|Enterobacter hormaechei
MKISEFLHLALPEEQWLPTISGVLRQFAEEECYVYERQPCWYLGKGCLAR
LHINADGTQATFIDDAGEQQWAVDSITDCARRFMAHPQVKGRRVYGQVGF
NFAAHARGIAFNAGEWPLLTLTVPREELIFEKGNVTVYADSADGGRRLCE
WVKEASTTTQNAPLAVDTALNGEAYKQQVARAVAEIRRGEYVKVIVSRAI
PLPSRIDMPATLLYGRQANTPVRSFMFRQEGREALGFSPELVMSVTGNKV
VTEPLAGTRDRMGNPEHNKAKEAELLHDSKEVLEHILSVKEAIAELEAVC
LPGSVVVEDLMSVRQRGSVQHLGSGVSGQLAENKDAWDAFTVLFPSITAS
GIPKNAALNAIMQIEKTPRELYSGAILLLDDMRFDAALVLRSVFQDSQRC
WIQAGAGIIAQSTPERELTETREKLASIAPYLMV
(SEQ ID NO: 4) ABV12066|Citrobacter koseri ATCC
BAA-895
MKISEFLHLALPEEQWLPTISGVLRQFAEEECYVYERQPCWYLGKGCQAR
LHINADGTQATFIDDAGEQKWAVDSIADCARRFMAHPQVKGRRVYGQVGF
NFAAHARGIAFNAGEWPLLTLTVPREELIFEKGNVTVYADSADGCRRLCE
WVKEAGTTTQNAPLAVDTALNGEAYKQQVVRAVAEIRRGEYVKVIVSRAI
PLPSRIDMPATLLYGRQANTPVRSFMFRQEGREALGFSPELVMSVTGNKV
VTEPLAGTRDRMGNPEHNKAKEAELLHDSKEVLEHILSVKEAIAELEAVC
QPGSVVVEDLMSVRQRGSVQHLGSGVSGQLAENKDAWDAFTVLFPSITAS
GIPKNAALNAIMQIENTPRELYSGAILLLDDTRFDAALVLRSVFQDSQRS
WIQAGAGIIAQSTPERELTETREKLASIAPYLMV
(SEQ ID NO: 5) AFQ65037|Klebsiella pneumoniae
subsp. pneumoniae 1084
MKISEFLHLALPEEQWLPTISGVLRQFAEEECYVYERQPCWYLGKGCQAR
LHINADGTQATFIDDAGEQKWAVDSIADCARRFMAHPQVKGRRVYGQVGF
NFAAHARGIAFDAGEWPLLTLTVPREELIFEKGNVTVYADSADGCRRLCE
WVKEASTTTQNAPLAVDTALNGEAYKQQVARAVAEIRRGEYVKVIVSRAI
PLPSRIDMPATLLYGRQANTPVRSFMFRQEGREALGFSPELVMSVTGNKV
VTEPLAGTRDRMGNPEHNKAKEAELLHDSKEVLEHILSVKEAIAELEAVC
LPGSVVVEDLMSVRQRGSVQHLGSGVSGQLAENKDAWDAFTVLFPSITAS
GIPKNAALNAIMQIEKTPRELYSGAILLLDDTRFDAALVLRSVFQDSQRS
WIQAGAGIIAQSTPERELTETREKLASIAPYLMV
(SEQ ID NO: 6) CAB46570|Yersinia enterocolitica
MKISEFLHLALPEEQWLPTISGVLRQFAEEECYVYERPPCWYLGKGCQAR
LHINADGTQATFIDDAGEQKWAVDSIADCARRFMAHPQVKGRRVYGQVGF
NFAAHARGIAFNAGEWPLLTLTVPREELIFEKGNVTVYADSADGCRRLCE
WVKEASTTTQNAPLAVDTALNGEAYKQQVARAVAEIRRGEYVKVIVSRAI
PLPSRIDMPATLLYGRQANTPVRSFMFRQEGREALGFSPELVMSVTGNKV
VTEPLAGTRDRMGNPEHNKAKEAELLHDSKEVLEHILSVKEAIAELEAVC
LPGSVVVEDLMSVRQRGSVQHLGSGVSGQLAENKDAWDAFTVLFPSITAS
GIPKNAALNAIMQIEKTPRELYSGAILLLDDTRFDAALVLRSVFQDSQRC
WIQAGAGIIAQSTPERELTETREKLASIAPYLMV
(SEQ ID NO: 7) ACY62547|Yersinia pestis D182038
MKISEFLHLALPEEQWLPTISGVLRQFAEEECYVYERQPCWYLGKGCQAR
LHINADGTQATFIDDAGEQKWAVDSIADCARRFMAHPQVKGRRVYGQVGF
NFAAHARGIAFNAGEWPLLTLTVPREELIFEKGNVTVYADSADGCRRLCE
WVKEAGTTTQNAPLAVDTALNGEAYKQQVARAVAEIRRGEYVKVIVSRAI
PLPSRIDMPATLLYGRQANTPVRSFMFRQEGREALGFSPELVMSVTGNKV
VTEPLAGTRDRMGNPEHNKAKEAELLHDSKEVLEHILSVKEAIAELEAVC
QPGSVVVEDLMSVRQRGSVQHLGSGVSGQLAENKDAWDAFTVLFPSITAS
GIPKNAALNAIMQIEKTPRELYSGAILLLDDTRFDAALVLRSVFQDSQRC
WIQAGAGIIAQSTPERELTETREKLASIAPYLMV
(SEQ ID NO: 8) ACC88687|Yersinia
pseudotuberculosis PB1/+
MKISEFLHLALPEEQWLPTISGVLRQFAEEECYVYERQPCWYLGKGCQAR
LHINADGTQATFIDDAGEQKWAVDSIADCARRFMAHPQVKGRRVYGQVGF
NFAAHARGIAFNAGEWPLLTLTVPREELIFEKGNVTVYADSADGCRRLCE
WVKEAGTTTQNAPLAVDTALNGEAYKQQVARAVAEIRRGEYVKVIVSRAI
PLPSRIDMPATLLYGRQANTPVRSFMFRQEGREALGFSPELVMSVTGNKV
VTEPLAGTRDRMGNPEHNKAKEAELLHDSKEVLEHILSVKEAIAELEAVC
QPGSVVVEDLMSVRQRGSVQHLGSGVSGQLAENKDAWDAFTVLFPSITAS
GIPKNAALNAIMQIEKTPRELYSGAILLLDDTRFDAALVLRSVFQDSQRC
WIQAGAGIIAQSTPERELTETREKLASIAPYLMV
(SEQ ID NO: 9) ADN46761|Escherichia colt ABU 83972
MKISEFLHLALPEEQWLPTISGVLRQFAEEECYVYERQPCWYLGKGCQAR
LHINADGTQATFIDDAGEQKWAVDSIADCARRFMAHPQVKGRRVYGQVGF
NFAAHARGIAFNAGEWPLLTLTVPREELIFEKGNVTVYADSADGCRRLCE
WVKEAGTTTQNAPLAVDTALNGEAYKQQVARAVAEIRRGEYVKVIVSRAI
PLPSRIDMPATLLYGRQANTPVRSFMFRQEGREALGFSPELVMSVTGNKV
VTEPLAGTRDRMGNPEHNKAKEAELLHDSKEVLEHILSVKEAIAELEAVC
QPGSVVVEDLMSVRQRGSVQHLGSGVSGQLAENKDAWDAFTVLFPSITAS
GIPKNAALNAIMQIEKTPRELYSGAILLLDDTRFDAALVLRSVFQDSQRC
WIQAGAGIIAQSTPERELTETREKLASIAPYLMV
(SEQ ID NO: 10)Q7D785|MbtI|Mycobacterium
tuberculosis
MSELSVATGAVSTASSSIPMPAGVNPADLAAELAAVVTESVDEDYLLYEC
DGQWVLAAGVQAMVELDSDELRVIRDGVTRRQQWSGRPGAALGEAVDRLL
LETDQAFGWVAFEFGVHRYGLQQRLAPHTPLARVFSPRTRIMVSEKEIRL
FDAGIRHREAIDRLLATGVREVPQSRSVDVSDDPSGFRRRVAVAVDEIAA
GRYHKVILSRCVEVPFAIDFPLTYRLGRRHNTPVRSFLLQLGGIRALGYS
PELVTAVRADGVVITEPLAGTRALGRGPAIDRLARDDLESNSKEIVEHAI
SVRSSLEEITDIAEPGSAAVIDFMTVRERGSVQHLGSTIRARLDPSSDRM
AALEALFPAVTASGIPKAAGVEAIFRLDECPRGLYSGAVVMLSADGGLDA
ALTLRAAYQVGGRTWLRAGAGIIEESEPEREFEETCEKLSTLTPYLVARQ
(SEQ ID NO: 11)>gb|CP003683.1|:3157320-3158630
Klebsiella oxytoca E718, complete genome
ATGAAAATCAGTGAATTTTTACACCTGGCGTTACCAGAGGAGCAATGGCT
GCCGATGATTTCTGGCGTCTTACGTCAGTTCGGAGATGAAGAGTGCTATG
TCTATGAGCGCCAACCTTGCTGGTACATAGGTAGAGGATGCCAGGCGCAA
CTGCAGATTAATGCCGACGGCACCCAGGCGACATTCATTGATGATGCCGG
AGAACAAAAGTGGGCGGTAGATTCGATTACCGACTGCGCGCGTCGTTTTA
TGACGCATCCGCGGGTAAGAGGATGCCGGGTATATGGCCAGGTTGGGTTC
AACTTTGCGGCTCATGCGCGGGGAATGGCCTTTGATGCCGGCGAGTGGCC
GCTGCTAACGTTAACCGCTCCCCGTGAAGAACTTATTTTTGAGAAAGGAA
ATGTTACCGTTTACGCGGACTCCGCCGACGGATGCCGCCGTCTGTGCGAG
TGGGTAAAAGAGGTCGATACGGCGACACCATGCGGGCCAATGGTTGTGGA
TACCGCCCTGGACGGTGAAGCGTATAAACAGCAGGTTGCGCGCGCCGTTT
CGGCGATCCGCCGCGGCGATTACGTTAAAGTCATCGTCTCACGCGCGATT
CCTCTGCCAGCGCGTATTGATATGCCCGCTACGCTGCTATATGGACGACA
GGCCAACACGCCCACCCGTACGTTTATGTTTCGCCAGCAAGGACGCGAGG
CGCTGGGTTTTAGTCCGGAACTGGTGATGTCGGTGACGGGAAAGAAAGTG
GTCACTGAACCGCTGGCGGGCACCCGCGATCGCATGGGCGATATGGCGCA
TAATCAGGCAAATGAGAGGGAGCTGCTGCACGACGGTAAAGAGGTGCTTG
AACATATCCTCTCGGTCAAAGAAGCCATTGCCGAACTGGAGGCGGTTTGC
CAGCCCGGCAGCGTGGTGGTCGAGGATTTGATGTCGGTTCGCCAGCGCGG
CAGCGTCCAGCATCTGGGGTCTGGCGTCAGCGGCCAGCTCGCGGAAGATA
AGGATGCCTGGGATGCGTTTACCGTGCTGTTTCCGTCAATTACCGCGTCA
GGTATCCCTAAAAAAGCGGCCCTCAACGCGATCATGCAAATTGAAAAAAC
GCCGCGGGAGCTCTATTCAGGCGCAATCCTGCTGCTGGAAGATACGCGCT
TCGATGCGGCGTTAGTACTGCGTTCCGTGTTTCAGGATAGCCAGCGCTGC
TGGATACAGGCGGGGGCGGGTATCATCGAGCAATCTACGCCGGAGCGCGA
ACTGACGGAAACCCGGGAGAAATTAGCGAGCATTGCGCCCTATTTAAAGG
TGCCCGCGTGA
(SEQ ID NO: 12)>gb|CP004142.1|:601029-602329
Raoultella ornithinolytica B6, complete genome
ATGAAAATCAGTGAATTTTTACACCTGGCGCTACCAGAGGAACAATGGCT
GCCGACGATTTCTGGCGTCTTACGCCAGTTCGGAGATGAAGAGTGCTATG
TCTACGAGCGTCAACCCTGTTGGTATTTAGGTAAAGGATGCCTGGCACGG
TTGCACATTAATGCCGACGGAACGCAGGCGACATTCATTGATGATGCCGG
GGAGCAAAAATGGGCGGTGGATTCCATTGTCGACTGCGCGCGTCGTTTTA
TGGCGCATCCGCAGGTGCAAGGGCGTCGGGTATATGGGCAGGTTGGGTTC
AACTTTGCGGCGCATGCGCGGGGGATTGCCTTCGACGCCGGAGAGTGGCC
GCTGCTGACATTAACCGTTCCCCGGGAAGAGCTTGTGTTTGAAAAGGGAA
ATGTCACCGTTTATACGGACTCCGCTGAGGGATGTCGACGTCTGTGCGAG
TGGGTAAAAGAGGCCAGTACAACCACGCGAGGGGACTCCATGGCTGTGGA
TACCGCCCTCAATGGTGAGGTGTATAAACAACAGGTGGCGCGCGCCGTTG
CGGAGATCAGCCGGGGCGAATATGTCAAAGTGATTATCTCGCGCGCCATT
CCGCTGCCATCGCGGATTGATATGCCCGCCACCCTGTTATACGGGCGGCA
GGCAAACACGCCCACGCGGTCGTTTATGTTCCGCCAGCAAGGACGCGAAG
CGCTAGGGTTTAGCCCGGAGCTGGTGATGTCGGTAACGGGCAATAAAGTG
GTCACTGAACCGCTTGCGGGCACCCGCGATCGCATGGGAAGCCCGGAGCA
AAATAAGGCGAAAGAGACAGAGCTGCTGCACGACAGTAAAGAGGTGCTTG
AGCATATCCTTTCTGTCAAAGAAGCGCTTGCTGAACTGGCGGTGGTTTGC
CGGCCGGGCAGCGTGGTGGTTGAAGATTTAATGTCGGTCCGCAAGCGTGG
CAGCGTTCAGCATCTGGGGTCTGGCGTGAGCGGTCAGCTTGCGGAAAACA
AAGATGCCTGGGATGCATTTACCGTGCTGTTCCCGTCGATTACCGCCTCG
GGTATCCCTAAAAATGCTGCTCTGAACGCCATTATGCGAATTGAGAAGAC
CCCGCGAGAGCTCTATTCCGGCGCAATCCTGCTACTGGAAGATGCGCGCT
TCGATGCGGCGTTAGTCCTGCGTTCCGTATTTCAGGACAGTCAACGGTGC
TGGATACAGGCGGGAGCAGGGATCATTGCCCAATCTACGCCGGAACGTGA
ACTGACGGAAACCCGGGAGAAATTAGCGAGCATTGCGCCCTATCTCATGG
T
(SEQ ID NO: 13)>gi|284919674:44045-45349
Enterobacter hormaechei recombination hotspot
with five putative genomic islands, isolate 05-545
ATGAAAATCAGTGAATTTTTACACCTGGCGTTACCAGAGGAACAATGGCT
GCCGACGATTTCTGGCGTTTTACGCCAGTTCGCAGAAGAAGAGTGTTATG
TCTATGAGCGTCAACCCTGTTGGTATTTAGGCAAAGGGTGCCTGGCACGG
TTGCACATTAATGCCGACGGAACGCAGGCGACATTTATTGATGATGCCGG
GGAGCAACAATGGGCGGTGGATTCCATTACCGACTGCGCGCGTCGTTTTA
TGGCACATCCTCAGGTCAAAGGACGTCGGGTTTATGGACAGGTTGGGTTC
AACTTTGCGGCGCATGCGCGGGGGATTGCCTTTAACGCCGGGGAGTGGCC
GCTGCTGACGTTAACCGTTCCCCGTGAAGAACTTATTTTTGAAAAGGGAA
ATGTCACCGTTTATGCGGACTCCGCCGACGGGGGCCGACGTTTGTGTGAG
TGGGTAAAAGAGGCCAGTACAACGACGCAGAACGCACCACTGGCGGTGGA
TACCGCCCTCAATGGCGAGGCGTATAAACAACAGGTTGCACGCGCCGTTG
CGGAGATCCGCCGTGGCGAGTATGTCAAAGTGATTGTCTCGCGCGCTATT
CCCCTGCCATCGCGGATTGATATGCCCGCCACGCTGTTATACGGGCGGCA
GGCAAATACACCTGTGCGCTCGTTTATGTTCCGTCAGGAAGGACGCGAAG
CGCTGGGCTTTAGCCCGGAACTGGTGATGTCAGTGACGGGCAATAAAGTG
GTCACTGAACCGCTTGCGGGCACCCGCGATCGCATGGGAAACCCGGAGCA
TAATAAGGCGAAAGAGGCAGAACTGCTGCACGACAGTAAAGAGGTGCTTG
AGCATATCCTTTCTGTCAAAGAAGCGATTGCTGAACTGGAGGCCGTTTGC
CTGCCGGGCAGCGTGGTGGTTGAAGATTTAATGTCGGTTCGCCAGCGTGG
CAGCGTTCAGCATCTGGGGTCCGGCGTGAGCGGTCAGCTTGCGGAAAACA
AGGATGCCTGGGATGCGTTTACCGTGCTGTTTCCGTCGATTACCGCCTCA
GGTATCCCTAAAAACGCTGCCCTGAACGCGATTATGCAAATTGAGAAGAC
GCCGCGAGAGCTCTATTCCGGCGCAATTCTGCTGCTGGACGATATGCGCT
TCGATGCGGCGCTAGTTCTGCGTTCCGTATTTCAGGACAGCCAGCGCTGC
TGGATACAAGCGGGGGCGGGAATCATCGCGCAATCTACACCGGAACGCGA
ACTGACGGAAACCCGGGAGAAATTAGCGAGCATTGCGCCCTATTTAATGG
TGTAG
(SEQ ID NO: 14)>gb|CP000822.1|:916630-917934
Citrobacter koseri ATCC BAA-895, complete genome
ATGAAAATCAGTGAATTTTTACACCTGGCGTTACCAGAGGAACAATGGCT
GCCGACGATTTCTGGCGTTTTACGCCAGTTCGCAGAAGAAGAGTGTTATG
TCTATGAGCGTCAACCCTGTTGGTATTTAGGCAAAGGGTGCCAGGCACGG
TTGCACATTAATGCCGATGGAACGCAGGCGACATTTATTGATGATGCCGG
GGAGCAAAAATGGGCGGTGGATTCCATTGCCGACTGCGCGCGTCGTTTTA
TGGCGCATCCTCAGGTGAAAGGACGTCGGGTATATGGACAGGTCGGGTTC
AACTTTGCGGCGCATGCGCGGGGGATTGCCTTTAACGCCGGGGAGTGGCC
GCTGCTGACGTTAACCGTTCCCCGTGAAGAACTTATTTTTGAAAAGGGAA
ATGTCACCGTTTATGCGGACTCCGCCGACGGGTGCCGACGTCTGTGCGAG
TGGGTAAAAGAGGCCGGTACAACGACGCAGAACGCACCACTGGCGGTGGA
TACCGCCCTCAATGGTGAGGCGTATAAACAACAGGTTGTACGCGCCGTTG
CGGAGATCCGCCGTGGCGAGTATGTCAAAGTGATTGTCTCGCGCGCCATT
CCCCTGCCATCGCGGATTGATATGCCTGCCACGCTGTTATACGGTCGGCA
GGCAAACACGCCTGTGCGCTCGTTTATGTTCCGTCAGGAAGGACGCGAAG
CGCTGGGCTTTAGCCCGGAACTGGTGATGTCAGTGACGGGCAATAAAGTG
GTCACTGAACCGCTTGCGGGCACCCGCGATCGCATGGGAAACCCGGAGCA
TAATAAGGCGAAAGAGGCAGAACTGCTGCACGACAGTAAAGAGGTGCTTG
AGCATATCCTTTCTGTCAAAGAAGCTATTGCTGAACTGGAGGCCGTTTGC
CAGCCGGGCAGCGTGGTGGTTGAAGATTTAATGTCGGTTCGCCAGCGCGG
CAGCGTTCAGCATCTGGGGTCTGGCGTGAGCGGTCAGCTTGCGGAAAACA
AGGATGCCTGGGATGCGTTTACTGTATTGTTTCCGTCGATTACCGCCTCA
GGCATCCCTAAAAACGCTGCTCTGAACGCGATTATGCAAATTGAGAATAC
GCCGCGAGAGCTTTATTCCGGCGCAATTCTGCTGCTGGACGATACGCGCT
TCGATGCGGCGCTGGTCCTGCGTTCCGTATTTCAGGACAGCCAGCGCAGC
TGGATACAGGCGGGGGCGGGAATCATCGCGCAATCTACGCCGGAACGCGA
ACTGACGGAAACCCGGGAGAAATTAGCGAGCATTGCGCCCTATTTAATGG
TGTAG
(SEQ ID NO: 15)>gb|CP003785.1|:1846306-1847610
Klebsiella pneumoniae subsp. pneumoniae 1084,
complete genome
ATGAAAATCAGTGAATTTTTACACCTGGCGTTACCAGAGGAACAATGGCT
GCCGACGATTTCTGGCGTTTTACGCCAGTTCGCAGAAGAAGAGTGTTATG
TCTATGAGCGTCAACCCTGTTGGTATTTAGGCAAAGGGTGCCAGGCACGG
TTGCACATTAATGCCGATGGAACGCAGGCGACATTTATTGATGATGCCGG
GGAGCAAAAATGGGCGGTGGATTCCATTGCCGACTGCGCGCGTCGTTTTA
TGGCGCATCCTCAGGTGAAAGGACGTCGGGTATATGGACAGGTTGGGTTC
AACTTTGCGGCGCATGCGCGGGGGATTGCCTTTGACGCCGGGGAGTGGCC
GCTGCTGACGTTAACCGTTCCCCGTGAAGAACTTATTTTTGAAAAGGGAA
ATGTCACCGTTTATGCGGACTCCGCCGACGGGTGCCGACGTTTGTGCGAG
TGGGTAAAAGAGGCCAGTACAACGACGCAGAACGCACCACTGGCGGTGGA
TACCGCCCTCAATGGTGAGGCGTATAAACAACAGGTTGCGCGCGCCGTTG
CGGAGATCCGCCGTGGCGAGTATGTCAAAGTGATTGTCTCGCGCGCCATT
CCCCTGCCATCGCGGATTGATATGCCCGCCACGCTGTTATACGGGCGGCA
GGCAAACACGCCTGTGCGCTCGTTTATGTTCCGTCAGGAAGGACGCGAAG
CGCTGGGCTTTAGCCCGGAACTGGTGATGTCAGTGACGGGCAATAAAGTG
GTCACTGAACCGCTTGCGGGCACCCGCGATCGCATGGGAAACCCGGAGCA
TAATAAGGCGAAAGAGGCAGAACTGCTGCACGACAGTAAAGAGGTGCTTG
AGCATATCCTTTCTGTCAAAGAAGCGATTGCTGAACTGGAGGCCGTTTGC
CTGCCGGGCAGCGTGGTGGTTGAAGATTTAATGTCGGTTCGCCAGCGCGG
CAGCGTTCAGCATCTGGGGTCCGGCGTGAGCGGTCAGCTTGCGGAAAACA
AGGATGCCTGGGATGCGTTTACCGTGCTGTTTCCGTCGATTACCGCCTCA
GGTATCCCTAAAAACGCTGCCCTGAACGCGATTATGCAAATTGAGAAGAC
GCCGCGAGAGCTCTATTCCGGCGCAATCCTGCTGCTGGACGATACGCGCT
TTGATGCGGCGCTAGTCCTGCGTTCCGTATTTCAGGACAGCCAGCGCAGC
TGGATACAGGCGGGGGCGGGGATCATCGCGCAATCTACGCCGGAACGCGA
ACTGACGGAAACCCGGGAGAAATTAGCGAGCATTGCGCCCTATTTAATGG
TGTAG
(SEQ ID NO: 16)>Yersinia_enterocolitica_Irp9_
CAB46570
ATGAAAATCAGTGAATTTCTACACCTGGCGTTACCAGAGGAACAATGGCT
GCCGACGATTTCTGGCGTTTTACGCCAGTTCGCAGAAGAAGAGTGTTATG
TCTATGAGCGCCAACCCTGTTGGTATTTAGGCAAAGGGTGCCAGGCACGG
CTGCACATTAATGCCGACGGAACGCAGGCGACATTTATTGATGATGCCGG
GGAGCAAAAATGGGCGGTGGATTCCATTGCCGACTGCGCGCGTCGTTTTA
TGGCACATCCTCAGGTGAAAGGACGTCGGGTATATGGACAGGTTGGGTTC
AACTTTGCGGCGCATGCGCGGGGGATTGCCTTTAACGCCGGGGAGTGGCC
GCTGCTGACGTTAACCGTTCCCCGTGAAGAACTTATTTTTGAAAAGGGAA
ATGTCACCGTTTATGCGGACTCCGCCGACGGGTGCCGACGTTTGTGCGAG
TGGGTAAAAGAGGCCAGTACAACGACGCAGAACGCACCACTGGCGGTGGA
TACCGCCCTCAATGGTGAGGCGTATAAACAACAGGTTGCGCGCGCCGTTG
CGGAGATCCGCCGTGGCGAGTATGTCAAAGTGATTGTCTCGCGCGCCATT
CCCCTGCCATCGCGGATTGATATGCCCGCCACGCTGTTATACGGGCGGCA
GGCAAACACGCCAGTGCGCTCGTTTATGTTCCGTCAGGAAGGACGCGAAG
CGCTGGGCTTTAGCCCGGAACTGGTGATGTCAGTGACGGGCAATAAAGTG
GTTACTGAACCGCTTGCGGGCACCCGCGATCGCATGGGAAACCCGGAGCA
TAATAAGGCGAAAGAGGCAGAACTGCTGCACGATAGTAAAGAGGTGCTTG
AGCATATCCTTTCTGTCAAAGAAGCTATTGCTGAACTGGAGGCCGTTTGC
CTGCCGGGCAGCGTGGTGGTTGAAGATTTAATGTCGGTTCGCCAGCGCGG
CAGCGTTCAGCATCTGGGGTCTGGCGTGAGCGGTCAGCTTGCGGAAAACA
AGGATGCCTGGGATGCGTTTACCGTGCTGTTTCCGTCGATTACCGCCTCA
GGTATCCCTAAAAACGCTGCCCTGAACGCGATTATGCAAATTGAGAAGAC
GCCGCGAGAGCTTTATTCCGGCGCAATTCTGCTGCTGGACGATACGCGCT
TCGATGCGGCGCTAGTTCTGCGTTCCGTATTTCAGGACAGCCAGCGCTGC
TGGATACAGGCGGGGGCGGGAATCATCGCGCAATCTACACCGGAACGCGA
ACTGACGGAAACCCGGGAGAAATTAGCGAGCATTGCGCCCTATTTAATGG
TGTAG
(SEQ ID NO: 17)>gb|CP001589.1|:2368664-2369968
Yersinia pestis D182038, complete genome
ATGAAAATCAGTGAATTTCTACATCTGGCGTTACCAGAGGAACAATGGCT
ACCGACGATTTCTGGCGTTTTACGCCAGTTCGCAGAAGAAGAGTGTTATG
TCTATGAGCGTCAACCCTGTTGGTATTTAGGCAAAGGGTGCCAGGCACGG
CTGCACATTAATGCCGACGGAACGCAGGCGACATTTATTGATGATGCCGG
GGAGCAAAAATGGGCGGTGGATTCCATTGCCGACTGCGCGCGTCGTTTTA
TGGCGCATCCTCAGGTGAAAGGACGTCGGGTATATGGACAGGTTGGGTTC
AACTTTGCGGCGCATGCGCGGGGGATTGCCTTTAACGCCGGGGAGTGGCC
GCTGCTGACGTTAACCGTTCCCCGTGAAGAACTTATTTTTGAAAAGGGAA
ATGTCACCGTTTATGCGGACTCCGCCGACGGGTGCCGACGTTTGTGCGAG
TGGGTAAAAGAGGCCGGTACAACGACGCAGAACGCACCACTGGCGGTGGA
TACCGCCCTCAATGGTGAGGCATATAAACAACAGGTTGCGCGCGCCGTTG
CGGAGATCCGCCGTGGCGAGTATGTCAAAGTGATTGTCTCGCGCGCCATT
CCCCTGCCATCGCGGATTGATATGCCCGCCACGCTGTTATACGGGCGGCA
GGCAAACACACCTGTGCGCTCGTTTATGTTCCGTCAGGAAGGACGCGAAG
CGCTGGGCTTTAGCCCGGAACTGGTGATGTCAGTGACGGGCAATAAAGTG
GTCACTGAACCGCTTGCGGGCACCCGCGATCGCATGGGAAACCCGGAGCA
TAATAAGGCGAAAGAGGCAGAACTGCTGCACGACAGTAAAGAGGTGCTTG
AGCATATCCTTTCTGTCAAAGAAGCTATTGCTGAACTGGAGGCCGTTTGC
CAGCCGGGCAGCGTGGTGGTTGAAGATTTAATGTCGGTTCGCCAGCGCGG
CAGCGTTCAGCATCTGGGGTCTGGCGTGAGCGGTCAGCTTGCGGAAAACA
AGGATGCCTGGGATGCGTTTACCGTGCTGTTTCCGTCGATTACCGCCTCA
GGTATCCCTAAAAATGCTGCTCTGAACGCGATTATGCAAATTGAGAAGAC
GCCGCGAGAGCTTTATTCCGGCGCAATCCTGCTGCTGGACGATACGCGCT
TTGATGCGGCGCTAGTTCTGCGTTCCGTATTTCAGGATAGCCAGCGCTGC
TGGATACAGGCGGGGGCGGGAATCATCGCGCAATCTACGCCGGAACGCGA
ACTGACAGAAACCCGGGAGAAATTAGCGAGCATTGCGCCCTATTTAATGG
TGTAG
(SEQ ID NO: 18)>gb|CP001048.1|:1946829-1948133
Yersinia pseudotuberculosis PB1/+, complete genome
ATGAAAATCAGTGAATTTCTACATCTGGCGTTACCAGAGGAACAATGGCT
ACCGACGATTTCTGGCGTTTTACGCCAGTTCGCAGAAGAAGAGTGTTATG
TCTATGAGCGTCAACCCTGTTGGTATTTAGGCAAAGGGTGCCAGGCACGG
CTGCACATTAATGCCGACGGAACGCAGGCGACATTTATTGATGATGCCGG
GGAGCAAAAATGGGCGGTGGATTCCATTGCCGACTGCGCGCGTCGTTTTA
TGGCGCATCCTCAGGTGAAAGGACGTCGGGTATATGGACAGGTTGGGTTC
AACTTTGCGGCGCATGCGCGGGGGATTGCCTTTAACGCCGGGGAGTGGCC
GCTGCTGACGTTAACCGTTCCCCGTGAAGAACTTATTTTTGAAAAGGGAA
ATGTCACCGTTTATGCGGACTCCGCCGACGGGTGCCGACGTTTGTGCGAG
TGGGTAAAAGAGGCCGGTACAACGACGCAGAACGCACCACTGGCGGTGGA
TACCGCCCTCAATGGTGAGGCATATAAACAACAGGTTGCGCGCGCCGTTG
CGGAGATCCGCCGTGGCGAGTATGTCAAAGTGATTGTCTCGCGCGCCATT
CCCCTGCCATCGCGGATTGATATGCCCGCCACGCTGTTATACGGGCGGCA
GGCAAACACACCTGTGCGCTCGTTTATGTTCCGTCAGGAAGGACGCGAAG
CGCTGGGCTTTAGCCCGGAACTGGTGATGTCAGTGACGGGCAATAAAGTG
GTCACTGAACCGCTTGCGGGCACCCGCGATCGCATGGGAAACCCGGAGCA
TAATAAGGCGAAAGAGGCAGAACTGCTGCACGACAGTAAAGAGGTGCTTG
AGCATATCCTTTCTGTCAAAGAAGCTATTGCTGAACTGGAGGCCGTTTGC
CAGCCGGGCAGCGTGGTGGTTGAAGATTTAATGTCGGTTCGCCAGCGCGG
CAGCGTTCAGCATCTGGGGTCTGGCGTGAGCGGTCAGCTTGCGGAAAACA
AGGATGCCTGGGATGCGTTTACCGTGCTGTTTCCGTCGATTACCGCCTCA
GGTATCCCTAAAAATGCTGCTCTGAACGCGATTATGCAAATTGAGAAGAC
GCCGCGAGAGCTTTATTCCGGCGCAATCCTGCTGCTGGACGATACGCGCT
TTGATGCGGCGCTAGTTCTGCGTTCCGTATTTCAGGATAGCCAGCGCTGC
TGGATACAGGCGGGGGCGGGAATCATCGCGCAATCTACGCCGGAACGCGA
ACTGACAGAAACCCGGGAGAAATTAGCGAGCATTGCGCCCTATTTAATGG
TGTAG
(SEQ ID NO: 19)>gb|CP001671.1|:2195018-2196322
Escherichia coli ABU 83972, complete genome
ATGAAAATCAGTGAATTTCTACATCTGGCGTTACCAGAGGAACAATGGCT
ACCGACGATTTCTGGCGTTTTACGCCAGTTCGCAGAAGAAGAGTGTTATG
TCTATGAGCGTCAACCCTGTTGGTATTTAGGCAAAGGGTGCCAGGCACGG
CTGCACATTAATGCCGACGGAACGCAGGCGACATTTATTGATGATGCCGG
GGAGCAAAAATGGGCGGTGGATTCCATTGCCGACTGCGCGCGTCGTTTTA
TGGCGCATCCTCAGGTGAAAGGACGTCGGGTATATGGACAGGTTGGGTTC
AACTTTGCGGCGCATGCGCGGGGGATTGCCTTTAACGCCGGGGAGTGGCC
GCTGCTGACGTTAACCGTTCCCCGTGAAGAACTTATTTTTGAAAAGGGAA
ATGTCACCGTTTATGCGGACTCCGCCGACGGGTGCCGACGTTTGTGCGAG
TGGGTAAAAGAGGCCGGTACAACGACGCAGAACGCACCACTGGCGGTGGA
TACCGCCCTCAATGGTGAGGCATATAAACAACAGGTTGCGCGCGCCGTTG
CGGAGATCCGCCGTGGCGAGTATGTCAAAGTGATTGTCTCGCGCGCCATT
CCCCTGCCATCGCGGATTGATATGCCCGCCACGCTGTTATACGGGCGGCA
GGCAAACACGCCTGTGCGCTCGTTTATGTTCCGTCAGGAAGGACGCGAAG
CGCTGGGCTTTAGCCCGGAACTGGTGATGTCAGTGACGGGCAATAAAGTG
GTCACTGAACCGCTTGCGGGCACCCGCGATCGCATGGGAAACCCGGAGCA
TAATAAGGCGAAAGAGGCAGAACTGCTGCACGACAGTAAAGAGGTGCTTG
AGCATATCCTTTCTGTCAAAGAAGCTATTGCTGAACTGGAGGCCGTTTGC
CAGCCGGGCAGCGTGGTGGTTGAAGATTTAATGTCGGTTCGCCAGCGCGG
CAGCGTTCAGCATCTGGGGTCTGGCGTGAGCGGTCAGCTTGCGGAAAACA
AGGATGCCTGGGATGCGTTTACCGTGCTGTTTCCGTCGATTACCGCCTCA
GGTATCCCTAAAAATGCTGCTCTGAACGCGATTATGCAAATTGAGAAGAC
GCCGCGAGAGCTTTATTCCGGCGCAATCCTGCTGCTGGACGATACGCGCT
TTGATGCGGCGCTAGTTCTGCGTTCCGTATTTCAGGATAGCCAGCGCTGC
TGGATACAGGCGGGGGCGGGAATCATCGCGCAATCTACGCCGGAACGCGA
ACTGACAGAAACCCGGGAGAAATTAGCGAGCATTGCGCCCTATTTAATGG
TGTAG
SEQ ID NO: 20
ATGGCTTCCACTGCTCTCTCAAGCGCCATCGTCGGAACTTCATTCATCCG
TCGTTCCCCAGCTCCAATCAGTCTCCGTTCCCTTCCATCAGCCAACACAC
AATCCCTCTTCGGTCTCAAATCAGGCACCGCTCGTGGTGGACGTGTCACA
GCCATGGCTACATACAAGGTC
SEQ ID NO: 21
ATGGCTTCCACTGCTCTCTCAAGCGCCATCGTCGGAACTTCATTCATCCG
TCGTTCCCCAGCTCCAATCAGTCTCCGTTCCCTTCCATCAGCCAACACAC
AATCCCTCTTCGGTCTCAAATCAGGCACCGCTCGTGGTGGACGTGTCACA
GCCATGGCTACATACAAGGTCGTCGACATGAAAATCAGTGAATTTCTACA
CCTGGCGTTACCAGAGGAACAATGGCTGCCGACGATTTCTGGCGTTTTAC
GCCAGTTCGCAGAAGAAGAGTGTTATGTCTATGAGCGCCAACCCTGTTGG
TATTTAGGCAAAGGGTGCCAGGCACGGCTGCACATTAATGCCGACGGAAC
GCAGGCGACATTTATTGATGATGCCGGGGAGCAAAAATGGGCGGTGGATT
CCATTGCCGACTGCGCGCGTCGTTTTATGGCACATCCTCAGGTGAAAGGA
CGTCGGGTATATGGACAGGTTGGGTTCAACTTTGCGGCGCATGCGCGGGG
GATTGCCTTTAACGCCGGGGAGTGGCCGCTGCTGACGTTAACCGTTCCCC
GTGAAGAACTTATTTTTGAAAAGGGAAATGTCACCGTTTATGCGGACTCC
GCCGACGGGTGCCGACGTTTGTGCGAGTGGGTAAAAGAGGCCAGTACAAC
GACGCAGAACGCACCACTGGCGGTGGATACCGCCCTCAATGGTGAGGCGT
ATAAACAACAGGTTGCGCGCGCCGTTGCGGAGATCCGCCGTGGCGAGTAT
GTCAAAGTGATTGTCTCGCGCGCCATTCCCCTGCCATCGCGGATTGATAT
GCCCGCCACGCTGTTATACGGGCGGCAGGCAAACACGCCAGTGCGCTCGT
TTATGTTCCGTCAGGAAGGACGCGAAGCGCTGGGCTTTAGCCCGGAACTG
GTGATGTCAGTGACGGGCAATAAAGTGGTTACTGAACCGCTTGCGGGCAC
CCGCGATCGCATGGGAAACCCGGAGCATAATAAGGCGAAAGAGGCAGAAC
TGCTGCACGATAGTAAAGAGGTGCTTGAGCATATCCTTTCTGTCAAAGAA
GCTATTGCTGAACTGGAGGCCGTTTGCCTGCCGGGCAGCGTGGTGGTTGA
AGATTTAATGTCGGTTCGCCAGCGCGGCAGCGTTCAGCATCTGGGGTCTG
GCGTGAGCGGTCAGCTTGCGGAAAACAAGGATGCCTGGGATGCGTTTACC
GTGCTGTTTCCGTCGATTACCGCCTCAGGTATCCCTAAAAACGCTGCCCT
GAACGCGATTATGCAAATTGAGAAGACGCCGCGAGAGCTTTATTCCGGCG
CAATTCTGCTGCTGGACGATACGCGCTTCGATGCGGCGCTAGTTCTGCGT
TCCGTATTTCAGGACAGCCAGCGCTGCTGGATACAGGCGGGGGCGGGAAT
CATCGCGCAATCTACACCGGAACGCGAACTGACGGAAACCCGGGAGAAAT
TAGCGAGCATTGCGCCCTATTTAATGGTGTAG
SEQ ID NO: 22
CATGGAGTCAAAGATTCAAATAGAGGACCTAACAGAACTCGCCGTAAAGA
CTGGCGAACAGTTCATACAGAGTCTCTTACGACTCAATGACAAGAAGAAA
ATCTTCGTCAACATGGTGGAGCACGACACACTTGTCTACTCCAAAAATAT
CAAAGATACAGTCTCAGAAGACCAAAGGGCAATTGAGACTTTTCAACAAA
GGGTAATATCCGGAAACCTCCTCGGATTCCATTGCCCAGCTATCTGTCAC
TTTATTGTGAAGATAGTGGAAAAGGAAGGTGGCTCCTACAAATGCCATCA
TTGCGATAAAGGAAAGGCCATCGTTGAAGATGCCTCTGCCGACAGTGGTC
CCAAAGATGGACCCCCACCCACGAGGAGCATCGTGGAAAAAGAAGACGTT
CCAACCACGTCTTCAAAGCAAGTGGATTGATGTGATATCTCCACTGACGT
AAGGGATGACGCACAATCCCACTATCCTTCGCAAGACCCTTCCTCTATAT
AAGGAAGTTCATTTCATTTGGAGAGAACACGGGGGACTCTTGACCATGGT
AGATCTAAAATGGCTTCCACTGCTCTCTCAAGCGCCATCGTCGGAACTTC
ATTCATCCGTCGTTCCCCAGCTCCAATCAGTCTCCGTTCCCTTCCATCAG
CCAACACACAATCCCTCTTCGGTCTCAAATCAGGCACCGCTCGTGGTGGA
CGTGTCACAGCCATGGCTACATACAAGGTCGTCGACATGAAAATCAGTGA
ATTTCTACACCTGGCGTTACCAGAGGAACAATGGCTGCCGACGATTTCTG
GCGTTTTACGCCAGTTCGCAGAAGAAGAGTGTTATGTCTATGAGCGCCAA
CCCTGTTGGTATTTAGGCAAAGGGTGCCAGGCACGGCTGCACATTAATGC
CGACGGAACGCAGGCGACATTTATTGATGATGCCGGGGAGCAAAAATGGG
CGGTGGATTCCATTGCCGACTGCGCGCGTCGTTTTATGGCACATCCTCAG
GTGAAAGGACGTCGGGTATATGGACAGGTTGGGTTCAACTTTGCGGCGCA
TGCGCGGGGGATTGCCTTTAACGCCGGGGAGTGGCCGCTGCTGACGTTAA
CCGTTCCCCGTGAAGAACTTATTTTTGAAAAGGGAAATGTCACCGTTTAT
GCGGACTCCGCCGACGGGTGCCGACGTTTGTGCGAGTGGGTAAAAGAGGC
CAGTACAACGACGCAGAACGCACCACTGGCGGTGGATACCGCCCTCAATG
GTGAGGCGTATAAACAACAGGTTGCGCGCGCCGTTGCGGAGATCCGCCGT
GGCGAGTATGTCAAAGTGATTGTCTCGCGCGCCATTCCCCTGCCATCGCG
GATTGATATGCCCGCCACGCTGTTATACGGGCGGCAGGCAAACACGCCAG
TGCGCTCGTTTATGTTCCGTCAGGAAGGACGCGAAGCGCTGGGCTTTAGC
CCGGAACTGGTGATGTCAGTGACGGGCAATAAAGTGGTTACTGAACCGCT
TGCGGGCACCCGCGATCGCATGGGAAACCCGGAGCATAATAAGGCGAAAG
AGGCAGAACTGCTGCACGATAGTAAAGAGGTGCTTGAGCATATCCTTTCT
GTCAAAGAAGCTATTGCTGAACTGGAGGCCGTTTGCCTGCCGGGCAGCGT
GGTGGTTGAAGATTTAATGTCGGTTCGCCAGCGCGGCAGCGTTCAGCATC
TGGGGTCTGGCGTGAGCGGTCAGCTTGCGGAAAACAAGGATGCCTGGGAT
GCGTTTACCGTGCTGTTTCCGTCGATTACCGCCTCAGGTATCCCTAAAAA
CGCTGCCCTGAACGCGATTATGCAAATTGAGAAGACGCCGCGAGAGCTTT
ATTCCGGCGCAATTCTGCTGCTGGACGATACGCGCTTCGATGCGGCGCTA
GTTCTGCGTTCCGTATTTCAGGACAGCCAGCGCTGCTGGATACAGGCGGG
GGCGGGAATCATCGCGCAATCTACACCGGAACGCGAACTGACGGAAACCC
GGGAGAAATTAGCGAGCATTGCGCCCTATTTAATGGTGTAGGCTAGCAAG
GGCGAATTCCAGCACACTGGCGGCCGTTACTAGTAAAGGAGAAGAACTTT
TCACTGGAGTTGTCCCAATTCTTGTTGAATTAGATGGTGATGTTAATGGG
CACAAATTTTCTGTCAGTGGAGAGGGTGAAGGTGATGCAACATACGGAAA
ACTTACCCTTAAATTTATTTGCACTACTGGAAAACTACCTGTTCCGTGGC
CAACACTTGTCACTACTTTCTCTTATGGTGTTCAATGCTTTTCAAGATAC
CCAGATCATATGAAGCGGCACGACTTCTTCAAGAGCGCCATGCCTGAGGG
ATACGTGCAGGAGAGGACCATCTTCTTCAAGGACGACGGGAACTACAAGA
CACGTGCTGAAGTCAAGTTTGAGGGAGACACCCTCGTCAACAGGATCGAG
CTTAAGGGAATCGATTTCAAGGAGGACGGAAACATCCTCGGCCACAAGTT
GGAATACAACTACAACTCCCACAACGTATACATCATGGCCGACAAGCAAA
AGAACGGCATCAAAGCCAACTTCAAGACCCGCCACAACATCGAAGACGGC
GGCGTGCAACTCGCTGATCATTATCAACAAAATACTCCAATTGGCGATGG
CCCTGTCCTTTTACCAGACAACCATTACCTGTCCACACAATCTGCCCTTT
CGAAAGATCCCAACGAAAAGAGAGACCACATGGTCCTTCNTGAGTTTGTA
ACAGCTGCTGGGATTACACATGGCATGGATGAACTATACAAAGCTAGCCA
CCACCACCACCACCACGTGTGAATTGGTGACCAGCTCGAATTTCCCCGAT
CGTTCAAACATTTGGCAATAAAGTTTCTTAAGATTGAATCCTGTTGCCGG
TCTTGCGATGATTATCATATAATTTCTGTTGAATTACGTTAAGCATGTAA
TAATTAACATGTAATGCATGACGTTATTTATGAGATGGGTTTTTATGATT
AGAGTCCCGCAATTATACATTTAATACGCGATAGAAAACAAAATATAGCG
CGCAAACTAGGATAAATTATCGCGCGCGGTGTCATCTATGTTACTAGATC
GGG

What is claimed is:

1. A plant comprising a heterologous expression cassette, wherein the expression cassette comprises a promoter operably linked to a polynucleotide, wherein the polynucleotide encodes a fusion polypeptide comprising a chloroplast targeting peptide and a single enzyme that catalyzes the conversion of chorismate to salicylic acid.

2. The plant of claim 1, wherein the single enzyme catalyzes the direct conversion of chorismate to salicylic acid.

3. The plant of claim 1, wherein the single enzyme is a bacterial salicylic acid synthase.

4. The plant of claim 3, wherein the salicylic acid synthase is from a bacteria selected from the group consisting of a Yersinia, Mycobacterium tuberculosis, Escherichia coli, Klebsiella, Enterobacter, Citrobacter and Raoultella.

5. The plant of claim 4, wherein the enzyme is Yersinia enterocolitica Irp9.

6. The plant of claim 1, wherein the amount of SA in the plant is increased compared to a control plant that does not comprise the expression cassette.

7. The plant of claim 1, wherein the plant has enhanced stress tolerance compared to a control plant that does not comprise the expression cassette.

8. The plant of claim 7, wherein the plant's growth is not significantly affected, as compared to a control plant that does not comprise the expression cassette.

9. The plant of claim 7, wherein the plant has increased disease resistance, drought tolerance, heat tolerance or heavy metal tolerance compared to a control plant that does not comprise the expression cassette.

10. The plant of any of claim 1, wherein the plant is a Populus plant.

11. A plant cell or plant seed comprising a heterologous expression cassette, wherein the expression cassette comprises a promoter operably linked to a polynucleotide, wherein the polynucleotide encodes a fusion polypeptide comprising a chloroplast targeting peptide and a single enzyme that catalyzes the conversion of chorismate to salicylic acid.

12. The plant cell or seed of claim 11, wherein the single enzyme catalyzes the direct conversion of chorismate to salicylic acid.

13. The plant cell or seed of claim 11, wherein the single enzyme is a bacterial salicylic acid synthase.

14. The plant cell or seed of claim 13, wherein the salicylic acid synthase is from a bacteria selected from the group consisting of a Yersinia, Mycobacterium tuberculosis, Escherichia coli, Klebsiella, Enterobacter, Citrobacter and Raoultella.

15. The plant cell or seed of claim 14, wherein the enzyme is Yersinia enterocolitica Irp9.

16. The plant cell or seed of claim 11, wherein the amount of SA in the plant cell or seed is increased compared to a control plant cell or seed that does not have the expression cassette.

17. The plant cell or seed of claim 11, wherein the plant cell or seed is from a Populus plant.

18. A transgenic plant comprising the plant cell of claim 11.

19. A transgenic plant regenerated from the plant cell or plant seed of any of claim 11.

20. A method of producing the plant of claim 1, comprising:
a) transforming a plant cell or a plant seed with a heterologous expression cassette, wherein the expression cassette comprises a promoter operably linked to a polynucleotide, wherein the polynucleotide encodes a fusion polypeptide comprising a chloroplast targeting peptide and a single enzyme that catalyzes the conversion of chorismate to salicylic acid;
b) regenerating a transgenic plant from said transformed plant cell or plant seed.

21. A cell or a seed from the plant produced by the method of claim 20.

22. The plant produced by the method of claim 20.

23. A heterologous expression cassette, wherein the expression cassette comprises a promoter operably linked to a polynucleotide, wherein the polynucleotide encodes a fusion polypeptide comprising a chloroplast targeting peptide and a single enzyme that catalyzes the conversion of chorismate to salicylic acid.

24. A vector comprising the heterologous expression cassette of claim 23.

25. A host cell comprising the vector of claim 24.


SEQUENCE LISTING (47 sequences)

SEQ ID NO: 1 - protein, 436 aa, Klebsiella oxytoca
MKISEFLHLALPEEQWLPMISGVLRQFGDEECYVYERQPCWYIGRGCQAQLQINADGTQATFIDDAGEQKWAVDSITDCARRFMTHPRVRGCRVYGQVGFNFAAHARGMAFDAGEWPLLTLTAPREELIFEKGNVTVYADSADGCRRLCEWVKEVDTATPCGPMVVDTALDGEAYKQQVARAVSAIRRGDYVKVIVSRAIPLPARIDMPATLLYGRQANTPTRTFMFRQQGREALGFSPELVMSVTGKKVVTEPLAGTRDRMGDMAHNQANERELLHDGKEVLEHILSVKEAIAELEAVCQPGSVVVEDLMSVRQRGSVQHLGSGVSGQLAEDKDAWDAFTVLFPSITASGIPKKAALNAIMQIEKTPRELYSGAILLLEDTRFDAALVLRSVFQDSQRCWIQAGAGIIEQSTPERELTETREKLASIAPYLKVPA

SEQ ID NO: 2 - protein, 436 aa, Raoultella ornithinolytica B6
MKISEFLHLALPEEQWLPTISGVLRQFGDEECYVYERQPCWYLGKGCLARLHINADGTQATFIDDAGEQKWAVDSIVDCARRFMAHPQVQGRRVYGQVGFNFAAHARGIAFDAGEWPLLTLTVPREELVFEKGNVTVYTDSAEGCRRLCEWVKEASTTTRGDSMAVDTALNGEVYKQQVARAVAEISRGEYVKVIISRAIPLPSRIDMPATLLYGRQANTPTRSFMFRQQGREALGFSPELVMSVTGNKVVTEPLAGTRDRMGSPEQNKAKETELLHDSKEVLEHILSVKEALAELAVVCRPGSVVVEDLMSVRKRGSVQHLGSGVSGQLAENKDAWDAFTVLFPSITASGIPKNAALNAIMRIEKTPRELYSGAILLLEDARFDAALVLRSVFQDSQRCWIQAGAGIIAQSTPERELTETREKLASIAPYLMVSE

SEQ ID NO: 3 - protein, 434 aa, Enterobacter hormaechei
MKISEFLHLALPEEQWLPTISGVLRQFAEEECYVYERQPCWYLGKGCLARLHINADGTQATFIDDAGEQQWAVDSITDCARRFMAHPQVKGRRVYGQVGFNFAAHARGIAFNAGEWPLLTLTVPREELIFEKGNVTVYADSADGGRRLCEWVKEASTTTQNAPLAVDTALNGEAYKQQVARAVAEIRRGEYVKVIVSRAIPLPSRIDMPATLLYGRQANTPVRSFMFRQEGREALGFSPELVMSVTGNKVVTEPLAGTRDRMGNPEHNKAKEAELLHDSKEVLEHILSVKEAIAELEAVCLPGSVVVEDLMSVRQRGSVQHLGSGVSGQLAENKDAWDAFTVLFPSITASGIPKNAALNAIMQIEKTPRELYSGAILLLDDMRFDAALVLRSVFQDSQRCWIQAGAGIIAQSTPERELTETREKLASIAPYLMV

SEQ ID NO: 4 - protein, 434 aa, Citrobacter koseri ATCC BAA-895
MKISEFLHLALPEEQWLPTISGVLRQFAEEECYVYERQPCWYLGKGCQARLHINADGTQATFIDDAGEQKWAVDSIADCARRFMAHPQVKGRRVYGQVGFNFAAHARGIAFNAGEWPLLTLTVPREELIFEKGNVTVYADSADGCRRLCEWVKEAGTTTQNAPLAVDTALNGEAYKQQVVRAVAEIRRGEYVKVIVSRAIPLPSRIDMPATLLYGRQANTPVRSFMFRQEGREALGFSPELVMSVTGNKVVTEPLAGTRDRMGNPEHNKAKEAELLHDSKEVLEHILSVKEAIAELEAVCQPGSVVVEDLMSVRQRGSVQHLGSGVSGQLAENKDAWDAFTVLFPSITASGIPKNAALNAIMQIENTPRELYSGAILLLDDTRFDAALVLRSVFQDSQRSWIQAGAGIIAQSTPERELTETREKLASIAPYLMV

SEQ ID NO: 5 - protein, 434 aa, Klebsiella pneumoniae subsp. pneumoniae 1084
MKISEFLHLALPEEQWLPTISGVLRQFAEEECYVYERQPCWYLGKGCQARLHINADGTQATFIDDAGEQKWAVDSIADCARRFMAHPQVKGRRVYGQVGFNFAAHARGIAFDAGEWPLLTLTVPREELIFEKGNVTVYADSADGCRRLCEWVKEASTTTQNAPLAVDTALNGEAYKQQVARAVAEIRRGEYVKVIVSRAIPLPSRIDMPATLLYGRQANTPVRSFMFRQEGREALGFSPELVMSVTGNKVVTEPLAGTRDRMGNPEHNKAKEAELLHDSKEVLEHILSVKEAIAELEAVCLPGSVVVEDLMSVRQRGSVQHLGSGVSGQLAENKDAWDAFTVLFPSITASGIPKNAALNAIMQIEKTPRELYSGAILLLDDTRFDAALVLRSVFQDSQRSWIQAGAGIIAQSTPERELTETREKLASIAPYLMV

SEQ ID NO: 6 - protein, 434 aa, Yersinia enterocolitica
MKISEFLHLALPEEQWLPTISGVLRQFAEEECYVYERPPCWYLGKGCQARLHINADGTQATFIDDAGEQKWAVDSIADCARRFMAHPQVKGRRVYGQVGFNFAAHARGIAFNAGEWPLLTLTVPREELIFEKGNVTVYADSADGCRRLCEWVKEASTTTQNAPLAVDTALNGEAYKQQVARAVAEIRRGEYVKVIVSRAIPLPSRIDMPATLLYGRQANTPVRSFMFRQEGREALGFSPELVMSVTGNKVVTEPLAGTRDRMGNPEHNKAKEAELLHDSKEVLEHILSVKEAIAELEAVCLPGSVVVEDLMSVRQRGSVQHLGSGVSGQLAENKDAWDAFTVLFPSITASGIPKNAALNAIMQIEKTPRELYSGAILLLDDTRFDAALVLRSVFQDSQRCWIQAGAGIIAQSTPERELTETREKLASIAPYLMV

SEQ ID NO: 7 - protein, 434 aa, Yersinia pestis D182038
MKISEFLHLALPEEQWLPTISGVLRQFAEEECYVYERQPCWYLGKGCQARLHINADGTQATFIDDAGEQKWAVDSIADCARRFMAHPQVKGRRVYGQVGFNFAAHARGIAFNAGEWPLLTLTVPREELIFEKGNVTVYADSADGCRRLCEWVKEAGTTTQNAPLAVDTALNGEAYKQQVARAVAEIRRGEYVKVIVSRAIPLPSRIDMPATLLYGRQANTPVRSFMFRQEGREALGFSPELVMSVTGNKVVTEPLAGTRDRMGNPEHNKAKEAELLHDSKEVLEHILSVKEAIAELEAVCQPGSVVVEDLMSVRQRGSVQHLGSGVSGQLAENKDAWDAFTVLFPSITASGIPKNAALNAIMQIEKTPRELYSGAILLLDDTRFDAALVLRSVFQDSQRCWIQAGAGIIAQSTPERELTETREKLASIAPYLMV

SEQ ID NO: 8 - protein, 434 aa, Yersinia pseudotuberculosis PB1/+; identical to SEQ ID NO: 7

SEQ ID NO: 9 - protein, 434 aa, Escherichia coli ABU 83972; identical to SEQ ID NO: 7

SEQ ID NO: 10 - protein, 450 aa, Mycobacterium tuberculosis
MSELSVATGAVSTASSSIPMPAGVNPADLAAELAAVVTESVDEDYLLYECDGQWVLAAGVQAMVELDSDELRVIRDGVTRRQQWSGRPGAALGEAVDRLLLETDQAFGWVAFEFGVHRYGLQQRLAPHTPLARVFSPRTRIMVSEKEIRLFDAGIRHREAIDRLLATGVREVPQSRSVDVSDDPSGFRRRVAVAVDEIAAGRYHKVILSRCVEVPFAIDFPLTYRLGRRHNTPVRSFLLQLGGIRALGYSPELVTAVRADGVVITEPLAGTRALGRGPAIDRLARDDLESNSKEIVEHAISVRSSLEEITDIAEPGSAAVIDFMTVRERGSVQHLGSTIRARLDPSSDRMAALEALFPAVTASGIPKAAGVEAIFRLDECPRGLYSGAVVMLSADGGLDAALTLRAAYQVGGRTWLRAGAGIIEESEPEREFEETCEKLSTLTPYLVARQ

SEQ ID NO: 11 - DNA, 1311 bp, Klebsiella oxytoca E718
atgaaaatcagtgaatttttacacctggcgttaccagaggagcaatggctgccgatgatttctggcgtcttacgtcagttcggagatgaagagtgctatgtctatgagcgccaaccttgctggtacataggtagaggatgccaggcgcaactgcagattaatgccgacggcacccaggcgacattcattgatgatgccggagaacaaaagtgggcggtagattcgattaccgactgcgcgcgtcgttttatgacgcatccgcgggtaagaggatgccgggtatatggccaggttgggttcaactttgcggctcatgcgcggggaatggcctttgatgccggcgagtggccgctgctaacgttaaccgctccccgtgaagaacttatttttgagaaaggaaatgttaccgtttacgcggactccgccgacggatgccgccgtctgtgcgagtgggtaaaagaggtcgatacggcgacaccatgcgggccaatggttgtggataccgccctggacggtgaagcgtataaacagcaggttgcgcgcgccgtttcggcgatccgccgcggcgattacgttaaagtcatcgtctcacgcgcgattcctctgccagcgcgtattgatatgcccgctacgctgctatatggacgacaggccaacacgcccacccgtacgtttatgtttcgccagcaaggacgcgaggcgctgggttttagtccggaactggtgatgtcggtgacgggaaagaaagtggtcactgaaccgctggcgggcacccgcgatcgcatgggcgatatggcgcataatcaggcaaatgagagggagctgctgcacgacggtaaagaggtgcttgaacatatcctctcggtcaaagaagccattgccgaactggaggcggtttgccagcccggcagcgtggtggtcgaggatttgatgtcggttcgccagcgcggcagcgtccagcatctggggtctggcgtcagcggccagctcgcggaagataaggatgcctgggatgcgtttaccgtgctgtttccgtcaattaccgcgtcaggtatccctaaaaaagcggccctcaacgcgatcatgcaaattgaaaaaacgccgcgggagctctattcaggcgcaatcctgctgctggaagatacgcgcttcgatgcggcgttagtactgcgttccgtgtttcaggatagccagcgctgctggatacaggcgggggcgggtatcatcgagcaatctacgccggagcgcgaactgacggaaacccgggagaaattagcgagcattgcgccctatttaaaggtgcccgcgtga

SEQ ID NO: 12 - DNA, 1301 bp, Raoultella ornithinolytica B6
atgaaaatcagtgaatttttacacctggcgctaccagaggaacaatggctgccgacgatttctggcgtcttacgccagttcggagatgaagagtgctatgtctacgagcgtcaaccctgttggtatttaggtaaaggatgcctggcacggttgcacattaatgccgacggaacgcaggcgacattcattgatgatgccggggagcaaaaatgggcggtggattccattgtcgactgcgcgcgtcgttttatggcgcatccgcaggtgcaagggcgtcgggtatatgggcaggttgggttcaactttgcggcgcatgcgcgggggattgccttcgacgccggagagtggccgctgctgacattaaccgttccccgggaagagcttgtgtttgaaaagggaaatgtcaccgtttatacggactccgctgagggatgtcgacgtctgtgcgagtgggtaaaagaggccagtacaaccacgcgaggggactccatggctgtggataccgccctcaatggtgaggtgtataaacaacaggtggcgcgcgccgttgcggagatcagccggggcgaatatgtcaaagtgattatctcgcgcgccattccgctgccatcgcggattgatatgcccgccaccctgttatacgggcggcaggcaaacacgcccacgcggtcgtttatgttccgccagcaaggacgcgaagcgctagggtttagcccggagctggtgatgtcggtaacgggcaataaagtggtcactgaaccgcttgcgggcacccgcgatcgcatgggaagcccggagcaaaataaggcgaaagagacagagctgctgcacgacagtaaagaggtgcttgagcatatcctttctgtcaaagaagcgcttgctgaactggcggtggtttgccggccgggcagcgtggtggttgaagatttaatgtcggtccgcaagcgtggcagcgttcagcatctggggtctggcgtgagcggtcagcttgcggaaaacaaagatgcctgggatgcatttaccgtgctgttcccgtcgattaccgcctcgggtatccctaaaaatgctgctctgaacgccattatgcgaattgagaagaccccgcgagagctctattccggcgcaatcctgctactggaagatgcgcgcttcgatgcggcgttagtcctgcgttccgtatttcaggacagtcaacggtgctggatacaggcgggagcagggatcattgcccaatctacgccggaacgtgaactgacggaaacccgggagaaattagcgagcattgcgccctatctcatggt

SEQ ID NO: 13 - DNA, 1305 bp, Enterobacter hormaechei
atgaaaatcagtgaatttttacacctggcgttaccagaggaacaatggctgccgacgatttctggcgttttacgccagttcgcagaagaagagtgttatgtctatgagcgtcaaccctgttggtatttaggcaaagggtgcctggcacggttgcacattaatgccgacggaacgcaggcgacatttattgatgatgccggggagcaacaatgggcggtggattccattaccgactgcgcgcgtcgttttatggcacatcctcaggtcaaaggacgtcgggtttatggacaggttgggttcaactttgcggcgcatgcgcgggggattgcctttaacgccggggagtggccgctgctgacgttaaccgttccccgtgaagaacttatttttgaaaagggaaatgtcaccgtttatgcggactccgccgacgggggccgacgtttgtgtgagtgggtaaaagaggccagtacaacgacgcagaacgcaccactggcggtggataccgccctcaatggcgaggcgtataaacaacaggttgcacgcgccgttgcggagatccgccgtggcgagtatgtcaaagtgattgtctcgcgcgctattcccctgccatcgcggattgatatgcccgccacgctgttatacgggcggcaggcaaatacacctgtgcgctcgtttatgttccgtcaggaaggacgcgaagcgctgggctttagcccggaactggtgatgtcagtgacgggcaataaagtggtcactgaaccgcttgcgggcacccgcgatcgcatgggaaacccggagcataataaggcgaaagaggcagaactgctgcacgacagtaaagaggtgcttgagcatatcctttctgtcaaagaagcgattgctgaactggaggccgtttgcctgccgggcagcgtggtggttgaagatttaatgtcggttcgccagcgtggcagcgttcagcatctggggtccggcgtgagcggtcagcttgcggaaaacaaggatgcctgggatgcgtttaccgtgctgtttccgtcgattaccgcctcaggtatccctaaaaacgctgccctgaacgcgattatgcaaattgagaagacgccgcgagagctctattccggcgcaattctgctgctggacgatatgcgcttcgatgcggcgctagttctgcgttccgtatttcaggacagccagcgctgctggatacaagcgggggcgggaatcatcgcgcaatctacaccggaacgcgaactgacggaaacccgggagaaattagcgagcattgcgccctatttaatggtgtag

SEQ ID NO: 14 - DNA, 1305 bp, Citrobacter koseri ATCC BAA-895
atgaaaatcagtgaatttttacacctggcgttaccagaggaacaatggctgccgacgatttctggcgttttacgccagttcgcagaagaagagtgttatgtctatgagcgtcaaccctgttggtatttaggcaaagggtgccaggcacggttgcacattaatgccgatggaacgcaggcgacatttattgatgatgccggggagcaaaaatgggcggtggattccattgccgactgcgcgcgtcgttttatggcgcatcctcaggtgaaaggacgtcgggtatatggacaggtcgggttcaactttgcggcgcatgcgcgggggattgcctttaacgccggggagtggccgctgctgacgttaaccgttccccgtgaagaacttatttttgaaaagggaaatgtcaccgtttatgcggactccgccgacgggtgccgacgtctgtgcgagtgggtaaaagaggccggtacaacgacgcagaacgcaccactggcggtggataccgccctcaatggtgaggcgtataaacaacaggttgtacgcgccgttgcggagatccgccgtggcgagtatgtcaaagtgattgtctcgcgcgccattcccctgccatcgcggattgatatgcctgccacgctgttatacggtcggcaggcaaacacgcctgtgcgctcgtttatgttccgtcaggaaggacgcgaagcgctgggctttagcccggaactggtgatgtcagtgacgggcaataaagtggtcactgaaccgcttgcgggcacccgcgatcgcatgggaaacccggagcataataaggcgaaagaggcagaactgctgcacgacagtaaagaggtgcttgagcatatcctttctgtcaaagaagctattgctgaactggaggccgtttgccagccgggcagcgtggtggttgaagatttaatgtcggttcgccagcgcggcagcgttcagcatctggggtctggcgtgagcggtcagcttgcggaaaacaaggatgcctgggatgcgtttactgtattgtttccgtcgattaccgcctcaggcatccctaaaaacgctgctctgaacgcgattatgcaaattgagaatacgccgcgagagctttattccggcgcaattctgctgctggacgatacgcgcttcgatgcggcgctggtcctgcgttccgtatttcaggacagccagcgcagctggatacaggcgggggcgggaatcatcgcgcaatctacgccggaacgcgaactgacggaaacccgggagaaattagcgagcattgcgccctatttaatggtgtag

SEQ ID NO: 15 - DNA, 1305 bp, Klebsiella pneumoniae subsp. pneumoniae 1084
atgaaaatcagtgaatttttacacctggcgttaccagaggaacaatggctgccgacgatttctggcgttttacgccagttcgcagaagaagagtgttatgtctatgagcgtcaaccctgttggtatttaggcaaagggtgccaggcacggttgcacattaatgccgatggaacgcaggcgacatttattgatgatgccggggagcaaaaatgggcggtggattccattgccgactgcgcgcgtcgttttatggcgcatcctcaggtgaaaggacgtcgggtatatggacaggttgggttcaactttgcggcgcatgcgcgggggattgcctttgacgccggggagtggccgctgctgacgttaaccgttccccgtgaagaacttatttttgaaaagggaaatgtcaccgtttatgcggactccgccgacgggtgccgacgtttgtgcgagtgggtaaaagaggccagtacaacgacgcagaacgcaccactggcggtggataccgccctcaatggtgaggcgtataaacaacaggttgcgcgcgccgttgcggagatccgccgtggcgagtatgtcaaagtgattgtctcgcgcgccattcccctgccatcgcggattgatatgcccgccacgctgttatacgggcggcaggcaaacacgcctgtgcgctcgtttatgttccgtcaggaaggacgcgaagcgctgggctttagcccggaactggtgatgtcagtgacgggcaataaagtggtcactgaaccgcttgcgggcacccgcgatcgcatgggaaacccggagcataataaggcgaaagaggcagaactgctgcacgacagtaaagaggtgcttgagcatatcctttctgtcaaagaagcgattgctgaactggaggccgtttgcctgccgggcagcgtggtggttgaagatttaatgtcggttcgccagcgcggcagcgttcagcatctggggtccggcgtgagcggtcagcttgcggaaaacaaggatgcctgggatgcgtttaccgtgctgtttccgtcgattaccgcctcaggtatccctaaaaacgctgccctgaacgcgattatgcaaattgagaagacgccgcgagagctctattccggcgcaatcctgctgctggacgatacgcgctttgatgcggcgctagtcctgcgttccgtatttcaggacagccagcgcagctggatacaggcgggggcggggatcatcgcgcaatctacgccggaacgcgaactgacggaaacccgggagaaattagcgagcattgcgccctatttaatggtgtag

SEQ ID NO: 16 - DNA, 1305 bp, Yersinia enterocolitica
atgaaaatcagtgaatttctacacctggcgttaccagaggaacaatggctgccgacgatttctggcgttttacgccagttcgcagaagaagagtgttatgtctatgagcgccaaccctgttggtatttaggcaaagggtgccaggcacggctgcacattaatgccgacggaacgcaggcgacatttattgatgatgccggggagcaaaaatgggcggtggattccattgccgactgcgcgcgtcgttttatggcacatcctcaggtgaaaggacgtcgggtatatggacaggttgggttcaactttgcggcgcatgcgcgggggattgcctttaacgccggggagtggccgctgctgacgttaaccgttccccgtgaagaacttatttttgaaaagggaaatgtcaccgtttatgcggactccgccgacgggtgccgacgtttgtgcgagtgggtaaaagaggccagtacaacgacgcagaacgcaccactggcggtggataccgccctcaatggtgaggcgtataaacaacaggttgcgcgcgccgttgcggagatccgccgtggcgagtatgtcaaagtgattgtctcgcgcgccattcccctgccatcgcggattgatatgcccgccacgctgttatacgggcggcaggcaaacacgccagtgcgctcgtttatgttccgtcaggaaggacgcgaagcgctgggctttagcccggaactggtgatgtcagtgacgggcaataaagtggttactgaaccgcttgcgggcacccgcgatcgcatgggaaacccggagcataataaggcgaaagaggcagaactgctgcacgatagtaaagaggtgcttgagcatatcctttctgtcaaagaagctattgctgaactggaggccgtttgcctgccgggcagcgtggtggttgaagatttaatgtcggttcgccagcgcggcagcgttcagcatctggggtctggcgtgagcggtcagcttgcggaaaacaaggatgcctgggatgcgtttaccgtgctgtttccgtcgattaccgcctcaggtatccctaaaaacgctgccctgaacgcgattatgcaaattgagaagacgccgcgagagctttattccggcgcaattctgctgctggacgatacgcgcttcgatgcggcgctagttctgcgttccgtatttcaggacagccagcgctgctggatacaggcgggggcgggaatcatcgcgcaatctacaccggaacgcgaactgacggaaacccgggagaaattagcgagcattgcgccctatttaatggtgtag

SEQ ID NO: 17 - DNA, 1305 bp, Yersinia pestis D182038
atgaaaatcagtgaatttctacatctggcgttaccagaggaacaatggctaccgacgatttctggcgttttacgccagttcgcagaagaagagtgttatgtctatgagcgtcaaccctgttggtatttaggcaaagggtgccaggcacggctgcacattaatgccgacggaacgcaggcgacatttattgatgatgccggggagcaaaaatgggcggtggattccattgccgactgcgcgcgtcgttttatggcgcatcctcaggtgaaaggacgtcgggtatatggacaggttgggttcaactttgcggcgcatgcgcgggggattgcctttaacgccggggagtggccgctgctgacgttaaccgttccccgtgaagaacttatttttgaaaagggaaatgtcaccgtttatgcggactccgccgacgggtgccgacgtttgtgcgagtgggtaaaagaggccggtacaacgacgcagaacgcaccactggcggtggataccgccctcaatggtgaggcatataaacaacaggttgcgcgcgccgttgcggagatccgccgtggcgagtatgtcaaagtgattgtctcgcgcgccattcccctgccatcgcggattgatatgcccgccacgctgttatacgggcggcaggcaaacacacctgtgcgctcgtttatgttccgtcaggaaggacgcgaagcgctgggctttagcccggaactggtgatgtcagtgacgggcaataaagtggtcactgaaccgcttgcgggcacccgcgatcgcatgggaaacccggagcataataaggcgaaagaggcagaactgctgcacgacagtaaagaggtgcttgagcatatcctttctgtcaaagaagctattgctgaactggaggccgtttgccagccgggcagcgtggtggttgaagatttaatgtcggttcgccagcgcggcagcgttcagcatctggggtctggcgtgagcggtcagcttgcggaaaacaaggatgcctgggatgcgtttaccgtgctgtttccgtcgattaccgcctcaggtatccctaaaaatgctgctctgaacgcgattatgcaaattgagaagacgccgcgagagctttattccggcgcaatcctgctgctggacgatacgcgctttgatgcggcgctagttctgcgttccgtatttcaggatagccagcgctgctggatacaggcgggggcgggaatcatcgcgcaatctacgccggaacgcgaactgacagaaacccgggagaaattagcgagcattgcgccctatttaatggtgtag

SEQ ID NO: 18 - DNA, 1305 bp, Yersinia pseudotuberculosis PB1/+; identical to SEQ ID NO: 17

SEQ ID NO: 19 - DNA, 1305 bp, Escherichia coli ABU 83972
atgaaaatcagtgaatttctacatctggcgttaccagaggaacaatggctaccgacgatttctggcgttttacgccagttcgcagaagaagagtgttatgtctatgagcgtcaaccctgttggtatttaggcaaagggtgccaggcacggctgcacattaatgccgacggaacgcaggcgacatttattgatgatgccggggagcaaaaatgggcggtggattccattgccgactgcgcgcgtcgttttatggcgcatcctcaggtgaaaggacgtcgggtatatggacaggttgggttcaactttgcggcgcatgcgcgggggattgcctttaacgccggggagtggccgctgctgacgttaaccgttccccgtgaagaacttatttttgaaaagggaaatgtcaccgtttatgcggactccgccgacgggtgccgacgtttgtgcgagtgggtaaaagaggccggtacaacgacgcagaacgcaccactggcggtggataccgccctcaatggtgaggcatataaacaacaggttgcgcgcgccgttgcggagatccgccgtggcgagtatgtcaaagtgattgtctcgcgcgccattcccctgccatcgcggattgatatgcccgccacgctgttatacgggcggcaggcaaacacgcctgtgcgctcgtttatgttccgtcaggaaggacgcgaagcgctgggctttagcccggaactggtgatgtcagtgacgggcaataaagtggtcactgaaccgcttgcgggcacccgcgatcgcatgggaaacccggagcataataaggcgaaagaggcagaactgctgcacgacagtaaagaggtgcttgagcatatcctttctgtcaaagaagctattgctgaactggaggccgtttgccagccgggcagcgtggtggttgaagatttaatgtcggttcgccagcgcggcagcgttcagcatctggggtctggcgtgagcggtcagcttgcggaaaacaaggatgcctgggatgcgtttaccgtgctgtttccgtcgattaccgcctcaggtatccctaaaaatgctgctctgaacgcgattatgcaaattgagaagacgccgcgagagctttattccggcgcaatcctgctgctggacgatacgcgctttgatgcggcgctagttctgcgttccgtatttcaggatagccagcgctgctggatacaggcgggggcgggaatcatcgcgcaatctacgccggaacgcgaactgacagaaacccgggagaaattagcgagcattgcgccctatttaatggtgtag

SEQ ID NO: 20 - DNA, 171 bp, Artificial sequence
  Synthetic construct
atggcttccactgctctctcaagcgccatcgtcggaacttcattcatccgtcgttccccagctccaatcagtctccgttcccttccatcagccaacacacaatccctcttcggtctcaaatcaggcaccgctcgtggtggacgtgtcacagccatggctacatacaaggtc

SEQ ID NO: 21 - DNA, 1482 bp, Artificial sequence
  Synthetic construct
atggcttccactgctctctcaagcgccatcgtcggaacttcattcatccgtcgttccccagctccaatcagtctccgttcccttccatcagccaacacacaatccctcttcggtctcaaatcaggcaccgctcgtggtggacgtgtcacagccatggctacatacaaggtcgtcgacatgaaaatcagtgaatttctacacctggcgttaccagaggaacaatggctgccgacgatttctggcgttttacgccagttcgcagaagaagagtgttatgtctatgagcgccaaccctgttggtatttaggcaaagggtgccaggcacggctgcacattaatgccgacggaacgcaggcgacatttattgatgatgccggggagcaaaaatgggcggtggattccattgccgactgcgcgcgtcgttttatggcacatcctcaggtgaaaggacgtcgggtatatggacaggttgggttcaactttgcggcgcatgcgcgggggattgcctttaacgccggggagtggccgctgctgacgttaaccgttccccgtgaagaacttatttttgaaaagggaaatgtcaccgtttatgcggactccgccgacgggtgccgacgtttgtgcgagtgggtaaaagaggccagtacaacgacgcagaacgcaccactggcggtggataccgccctcaatggtgaggcgtataaacaacaggttgcgcgcgccgttgcggagatccgccgtggcgagtatgtcaaagtgattgtctcgcgcgccattcccctgccatcgcggattgatatgcccgccacgctgttatacgggcggcaggcaaacacgccagtgcgctcgtttatgttccgtcaggaaggacgcgaagcgctgggctttagcccggaactggtgatgtcagtgacgggcaataaagtggttactgaaccgcttgcgggcacccgcgatcgcatgggaaacccggagcataataaggcgaaagaggcagaactgctgcacgatagtaaagaggtgcttgagcatatcctttctgtcaaagaagctattgctgaactggaggccgtttgcctgccgggcagcgtggtggttgaagatttaatgtcggttcgccagcgcggcagcgttcagcatctggggtctggcgtgagcggtcagcttgcggaaaacaaggatgcctgggatgcgtttaccgtgctgtttccgtcgattaccgcctcaggtatccctaaaaacgctgccctgaacgcgattatgcaaattgagaagacgccgcgagagctttattccggcgcaattctgctgctggacgatacgcgcttcgatgcggcgctagttctgcgttccgtatttcaggacagccagcgctgctggatacaggcgggggcgggaatcatcgcgcaatctacaccggaacgcgaactgacggaaacccgggagaaattagcgagcattgcgccctatttaatggtgtag

SEQ ID NO: 22 - DNA, 3103 bp, Artificial sequence
  Synthetic construct
catggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgaccatggtagatctaaaatggcttccactgctctctcaagcgccatcgtcggaacttcattcatccgtcgttccccagctccaatcagtctccgttcccttccatcagccaacacacaatccctcttcggtctcaaatcaggcaccgctcgtggtggacgtgtcacagccatggctacatacaaggtcgtcgacatgaaaatcagtgaatttctacacctggcgttaccagaggaacaatggctgccgacgatttctggcgttttacgccagttcgcagaagaagagtgttatgtctatgagcgccaaccctgttggtatttaggcaaagggtgccaggcacggctgcacattaatgccgacggaacgcaggcgacatttattgatgatgccggggagcaaaaatgggcggtggattccattgccgactgcgcgcgtcgttttatggcacatcctcaggtgaaaggacgtcgggtatatggacaggttgggttcaactttgcggcgcatgcgcgggggattgcctttaacgccggggagtggccgctgctgacgttaaccgttccccgtgaagaacttatttttgaaaagggaaatgtcaccgtttatgcggactccgccgacgggtgccgacgtttgtgcgagtgggtaaaagaggccagtacaacgacgcagaacgcaccactggcggtggataccgccctcaatggtgaggcgtataaacaacaggttgcgcgcgccgttgcggagatccgccgtggcgagtatgtcaaagtgattgtctcgcgcgccattcccctgccatcgcggattgatatgcccgccacgctgttatacgggcggcaggcaaacacgccagtgcgctcgtttatgttccgtcaggaaggacgcgaagcgctgggctttagcccggaactggtgatgtcagtgacgggcaataaagtggttactgaaccgcttgcgggcacccgcgatcgcatgggaaacccggagcataataaggcgaaagaggcagaactgctgcacgatagtaaagaggtgcttgagcatatcctttctgtcaaagaagctattgctgaactggaggccgtttgcctgccgggcagcgtggtggttgaagatttaatgtcggttcgccagcgcggcagcgttcagcatctggggtctggcgtgagcggtcagcttgcggaaaacaaggatgcctgggatgcgtttaccgtgctgtttccgtcgattaccgcctcaggtatccctaaaaacgctgccctgaacgcgattatgcaaattgagaagacgccgcgagagctttattccggcgcaattctgctgctggacgatacgcgcttcgatgcggcgctagttctgcgttccgtatttcaggacagccagcgctgctggatacaggcgggggcgggaatcatcgcgcaatctacaccggaacgcgaactgacggaaacccgggagaaattagcgagcattgcgccctatttaatggtgtaggctagcaagggcgaattccagcacactggcggccgttactagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccgtggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcntgagtttgtaacagctgctgggattacacatggcatggatgaactatacaaagctagccaccaccaccaccaccacgtgtgaattggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 23 - DNA, 25 bp, Artificial sequence
  Synthetic construct
gtcgacatgaaaatcagtgaatttc

SEQ ID NO: 24 - DNA, 25 bp, Artificial sequence
  Synthetic construct
agatctatgaaaatcagtgaatttc

SEQ ID NO: 25 - DNA, 25 bp, Artificial sequence
  Synthetic construct
gctagcctacaccattaaatagggc

SEQ ID NO: 26 - DNA, 24 bp, Artificial sequence
  Synthetic construct
atgcgtttaccgtgctgtttccgt

SEQ ID NO: 27 - DNA, 24 bp, Artificial sequence
  Synthetic construct
agggcgcaatgctcgctaatttct

SEQ ID NO: 28 - DNA, 27 bp, Artificial sequence
  Synthetic construct
agatctaaaatggcttccactgctctc

SEQ ID NO: 29 - DNA, 27 bp, Artificial sequence
  Synthetic construct
gtcgacgaccttgtatgtagccatggc

SEQ ID NO: 30 - DNA, 20 bp, Artificial sequence
  Synthetic construct
aacctcgccgagctgcttga

SEQ ID NO: 31 - DNA, 22 bp, Artificial sequence
  Synthetic construct
aggtcagtgtcgaggtcgtggt

SEQ ID NO: 32 - DNA, 24 bp, Artificial sequence
  Synthetic construct
tgccttggttagcccttccatttg

SEQ ID NO: 33 - DNA, 24 bp, Artificial sequence
  Synthetic construct
tgtcargtgcwtccgagcttcctt

SEQ ID NO: 34 - DNA, 24 bp, Artificial sequence
  Synthetic construct
accatgctagccattctcaacctg

SEQ ID NO: 35 - DNA, 24 bp, Artificial sequence
  Synthetic construct
ttagcactgtcaacgtgcattcca

SEQ ID NO: 36 - DNA, 24 bp, Artificial sequence
  Synthetic construct
gtgatcacgcttcraacaagccaa

SEQ ID NO: 37 - DNA, 26 bp, Artificial sequence
  Synthetic construct
taccatccatgtccaractgtgtgag

SEQ ID NO: 38 - DNA, 24 bp, Artificial sequence
  Synthetic construct
acgcgcaaaysgcaaagaagctga

SEQ ID NO: 39 - DNA, 26 bp, Artificial sequence
  Synthetic construct
tctgtttcarwgatcacytccaacgc

SEQ ID NO: 40 - DNA, 24 bp, Artificial sequence
  Synthetic construct
tggaggaaggaagaacgtcgatga

SEQ ID NO: 41 - DNA, 26 bp, Artificial sequence
  Synthetic construct
tgcrcttgttytcttagggacagagg

SEQ ID NO: 42 - DNA, 24 bp, Artificial sequence
  Synthetic construct
gacctkgtatcagtggattccctc

SEQ ID NO: 43 - DNA, 24 bp, Artificial sequence
  Synthetic construct
gaacagaggcacaagattaccagg

SEQ ID NO: 44 - DNA, 24 bp, Artificial sequence
  Synthetic construct
actgtgaggagatgcagaaacgca

SEQ ID NO: 45 - DNA, 24 bp, Artificial sequence
  Synthetic construct
gctgtgtcacgggcattcaatgyt

SEQ ID NO: 46 - DNA, 24 bp, Artificial sequence
  Synthetic construct
cgtgcagctggtctctrtatgtat

SEQ ID NO: 47 - DNA, 24 bp, Artificial sequence
  Synthetic construct
actgacacactggaagctccaaca